(19) United States
(12) Patent Application Publication
(10) Pub. No.: US 2013/0109758 A1
(43) Pub. Date: May 2, 2013

(54) BISPHENOL DERIVATIVE THERAPEUTICS AND METHODS FOR THEIR USE

(71) Applicants: Marianne D. Sadar, West Vancouver (CA); Nasrin R. Mawji, Burnaby (CA); Carmen Adriana Banuelos, Vancouver (CA); Raymond J. Andersen, Vancouver (CA); Javier Garcia Fernandez, Gijon (ES)

(72) Inventors: Marianne D. Sadar, West Vancouver (CA); Nasrin R. Mawji, Burnaby (CA); Carmen Adriana Banuelos, Vancouver (CA); Raymond J. Andersen, Vancouver (CA); Javier Garcia Fernandez, Gijon (ES)

(73) Assignees: THE UNIVERSITY OF BRITISH COLUMBIA, Vancouver, BC (CA); BRITISH COLUMBIA CANCER AGENCY BRANCH, Vancouver, BC (CA)

(21) Appl. No.: 13/520,731

(22) PCT Filed: Jan. 6, 2011
(86) PCT No.: PCT/CA2011/000021

§ 371 Date: Jan. 10, 2013

Related U.S. Application Data
(60) Provisional application No. 61/282,234, filed on Jan. 6, 2010.

(51) Int. Cl. C07C 49/84; C07C 323/20; C07C 43/295

(52) U.S. Cl. CPC C07C 49/84; C07C 43/295; C07C 323/20
USPC 514/687; 568/637; 514/721; 568/48; 514/712; 568/333

(57) ABSTRACT

This invention provides compounds having a structure of Formula (I). Uses of such compounds for treatment of various indications, including prostate cancer as well as methods of treatment involving such compounds are also provided.

### CROSS REFERENCE TO RELATED APPLICATIONS

This application claims the benefit of U.S. Provisional Patent Application Ser. No. 61/282,234 entitled “BISPHENOL DERIVATIVE THERAPEUTICS AND METHODS FOR THEIR USE” filed on 6 Jan. 2010.

### TECHNICAL FIELD

This invention relates to therapeutics, their uses and methods for the treatment of various indications, including various cancers. In particular the invention relates to therapies and methods of treatment for cancers such as prostate cancer, including all stages and androgen dependent, androgen-sensitive and androgen-independent (also referred to as hormone refractory, castration resistant, androgen deprivation resistant, androgen ablation resistant, androgen depletion-independent, castration-recurrent, anti-androgen-recurrent).

### BACKGROUND

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Sury 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration (androgen ablation).

Androgens also play a role in female cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit. Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate epithelial cells. Androgen ablation therapy causes a temporary reduction in tumor burden concomitant with a decrease in serum prostate-specific antigen (PSA). Unfortunately prostate cancer can eventually grow again in the absence of androgens (androgen-independent disease) (Huber et al 1987 Scand J. Urol Nephrol. 104, 33-39). Androgen-independent disease is biochemically characterized before the onset of symptoms by a rising titre of serum PSA (Miller et al 1992 J. Urol. 147, 956-961). Once the disease becomes androgen-independent most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains one or more transcriptional activation domains. Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et at 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et at 2002 A J. Biol. Chem. 277, 7076-7085; and Ueda et al 2002 B J. Biol. Chem. 277, 38087-38094). The AR may be activated in the absence of testicular androgens by alternative signal transduction pathways in androgen-independent disease, which is consistent with the finding that nuclear AR protein is present in secondary prostate cancer tumors (Kim et at 2002 Am. J. Pathol. 160, 219-226; and van der Kwast et at 1991 Inter. J. Cancer 48, 189-193).

Available inhibitors of the AR include nonsteroidal antiandrogens such as bicalutamide (Casodex™), nilutamide, and flutamide and the steroidal antiandrogen, cyproterone acetate. These antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)). These antiandrogens would also have no effect on the recently discovered AR splice variants that lack the ligand-binding domain (LBD) to result in a constitutively active receptor which promotes progression of androgen-independent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009).

Conventional therapy has concentrated on androgen-dependent activation of the AR through its C-terminal domain. Recent studies developing antagonists to the AR have concentrated on the C-terminus and specifically: 1) the allosteric pocket and AF-2 activity (Estebanez-Perpind et at 2007, PNAS 104, 16074-16079); 2) in silico “drug repurposing” procedure for identification of nonsteroidal antagonists (Bisson et al 2007, PNAS 104, 11927-11932); and coactivator or corepressor interactions (Chang et al 2005, Mol Endocrinology 19, 2478-2490; Hur et al 2004, PLoS Biol 2, E274; Estébanez-Perpiñá et at 2005, JBC 280, 8060-8068; He et al 2004, Mol Cell 16, 425-438).

The AR-NTD is also a target for drug development (e.g. WO 2000/001813), since the NTD plays a role in activation of the AR in the absence of androgens (Sadar, M. D. 1999 J. Biol. Chem. 274, 7777-7783; Sadar M D et at 1999 Endocr Relat Cancer. 6, 487-502; Ueda et at 2002 J. Biol. Chem. 277, 7076-7085; Ueda 2002 J. Biol. Chem. 277, 38087-38094; Blaszczyk et at 2004 Clin Cancer Res. 10, 1860-9; Dehm et al 2006 J Biol. Chem. 28, 27882-93; Gregory et al 2004 J Biol. Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinisic disorder in solution (Reid et at 2002 J. Biol. Chem. 277, 20079-20086) thereby hampering virtual docking drug discovery approaches.

### SUMMARY

This invention is based in part on the fortuitous discovery that compounds described herein modulate androgen receptor (AR) activity. Specifically, compounds identified herein, show inhibition of AR activity, which may be useful for blocking in vivo tumor growth in the presence and absence of androgens. The discovery was particularly fortuitous because the initial screen of marine invertebrate extracts was testing for inhibition of AR activity and some of the compounds identified in that initial screen were determined to have a structural resemblance to BADGE (Bisphenol A Diglycidic Ether). The resemblance to BADGE suggests that these compounds are most likely of industrial origin and were bioaccumulated by the sponge from the contaminated seawater. Accordingly, due to the known activities for Badge compounds, the present BADGE derivatives are very unlikely to have been screened in the assay under any other circumstances.

The compounds described herein may be used for in vivo or in vitro research uses (i.e. non-clinical) to investigate the mechanisms of orphan and nuclear receptors (including steroid receptors such as the androgen receptor). Furthermore, these compounds may be used individually or as part of a kit for in vivo or in vitro research to investigate signal transduction pathways and/or the activation of orphan and nuclear receptors using recombinant proteins, cells maintained in culture, and/or animal models.

This invention is also based in part on the surprising discovery that the compounds described herein, may also be used to modulate the androgen receptor activity either in vivo or in vitro for both research and therapeutic uses. The compounds may be used in an effective amount so that androgen receptor activity may be modulated. The androgen receptor may be mammalian. Alternatively, the androgen receptor may be human. In particular, the compounds may be used to inhibit the AR. The compounds modulatory activity may be used in either an in vivo or an in vitro model for the study of at least one of the following indications: prostate cancer, breast cancer, ovarian cancer, endometrial cancer, hair loss, acne, hirsutism, ovarian cysts, polycystic ovary disease, precocious puberty, and age-related macular degeneration. Furthermore, the compounds modulatory activity may be used for the treatment of at least one of the following indications: prostate cancer, breast cancer, ovarian cancer, endometrial cancer, hair loss, acne, hirsutism, ovarian cysts, polycystic ovary disease, precocious puberty (testoxicosis) and age-related macular degeneration. The indication for treatment may be prostate cancer. The prostate cancer may be androgen-independent prostate cancer. The prostate cancer may be androgen-dependent prostate cancer.

In accordance with one embodiment, there is provided a use of a compound having a structure of Formula I

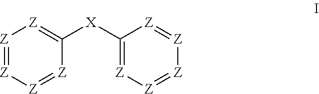

or a pharmaceutically acceptable salt thereof,
wherein: X may be a single bond, C═O, C(OH)₂, C(OR¹)₂, C(OH)(OR¹), C(OR¹)(OR²), O, S, SO, SO₂, C═NOH, C═N)R¹, CHNH₂, CHNHR¹, CHNHR², CHNR¹₂, CHNR²₂, or CHNR¹R²; each R¹ may independently be linear or branched, substituted or unsubstituted, saturated or unsaturated C₁-C₁₀ alkyl, and each R² may independently be C₁-C₁₀ acyl, or two of the R¹ groups may be joined to form a cyclic ketal, wherein the optional substituent may be selected from the group consisting of oxo, OJ′″, COOH, R³, OH, OR³, F, Cl, Br, I, NH₂, NHR³, NR³₂, CN, SH, SR³, SO₃H, SO₃R³, SO₂R³, OSO₃R³, OR⁶, CO₂R³, CONH₂, CONHR³, CONHR⁶, CONR³₂, NHR⁶, OPO₃H₃, CONR³R⁶, NR³R⁶, and NO₂, wherein each R³ may independently be unsubstituted C₁-C₁₀ alkyl and each R⁶ may independently be C₁-C₁₀ acyl; at least one Z of one aromatic ring may independently be C-Q, at least one Z of the other aromatic ring may independently be C-T, CF, CCl, CBr, CI, COH, CG¹, COG¹, CNH₂, CNHG¹, CNG¹₂, COSO₃H, COPO₃H₂, CSG¹, CSOG¹, or CSO₂G¹, and each remaining Z may independently be C-T, N, CH, CF, CCl, CBr, CI, COH, CG¹, COG¹, CNH₂, CNHG¹, CNG¹₂, COSO₃H, COPO₃H₂, CSG¹, CSOG¹, or CSO₂G¹; Q may be

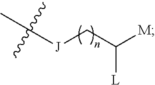

J may be G¹, O, CH₂, CHG¹, CG¹₂, S, NH, NG¹, SO, SO₂, or NR; M may be H, Cl, Br, CH₂Cl, CHCl₂, CCl₃, CH₂Br, CHBr₂, CBr₃, or C≡CH; L may be H or A-D; A may be O, S, NH, NG¹, N⁺H₂, or N⁺HG¹; D may be H, G¹, R,

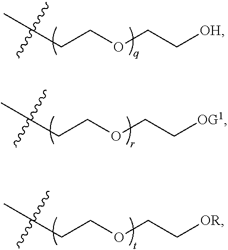

or a moiety selected from TABLE 1; each of q, r and t may independently be 0, 1, 2, 3, 4, 5, 6 or 7; n may be 0, 1, 2, 3, 4, 5, 6, 7 or 8; T may be

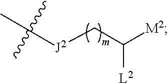

J² may be G¹, O, CH₂, CHG¹, CG¹₂, S, NH, NG¹, SO, SO₂, or NR; M² may be H, CH₃, Cl, Br, CH₂Cl, CHCl₂, CCl₃, CH₂Br, CHBr₂, CBr₃, CH₂OH, CH₂OJ″, G¹, CH₂OG¹, CH₂OR, CH₂OG¹, G¹′, G¹OG¹′, G¹OG¹OG¹″, CH₂SG¹, CH₂NH₂, CH₂NHG¹, CH₂NG¹₂, or C≡CH; L² may be H or A²-D²; A² may be O, S, SO, SO₂, NH, NG¹, N⁺H₂, or N⁺HG¹; D² may be H, G¹, R,

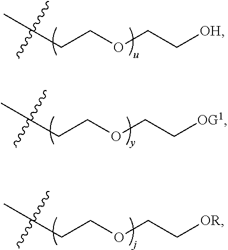

or a moiety selected from TABLE 1; each of u, y and j may independently be 0, 1, 2, 3, 4, 5, 6 or 7; m may be 0, 1, 2, 3, 4, 5, 6, 7 or 8; each of J″ and r may independently be a moiety selected from TABLE 1; each G¹ G¹′ and G¹″ may independently be linear or branched, or aromatic cyclic or non-aromatic cyclic, substituted or unsubstituted, saturated or unsaturated C₁-C₁₀ alkyl, wherein the optional substituent may be selected from the group consisting of oxo, Or, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, OR⁵, CO₂R⁴, CONH₂, CONHR⁴, CONHR⁵, CONR⁴₂, NHR⁵, OPO₃H₃, CONR⁴R⁵, NR⁴R⁵, and NO₂, wherein each R⁴ may independently be unsubstituted C₁-C₁₀ alkyl and each R⁵ may independently be C₁-C₁₀ acyl; and R may be C₁-C₁₀ acyl, for modulating androgen receptor (AR) activity.

In accordance with another embodiment, there is provided a use of a compound having a structure of Formula II

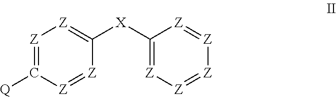

or a pharmaceutically acceptable salt thereof, wherein each X, Z, and Q may independently be defined as anywhere herein. In accordance with another embodiment, there is provided a use of a compound having a structure of Formula III

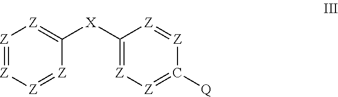

or a pharmaceutically acceptable salt thereof, wherein each X, Z, and Q may independently be defined as anywhere herein. In accordance with another embodiment, there is provided a use of a compound having a structure of Formula IV

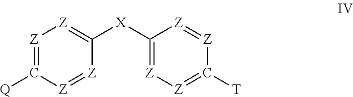

or a pharmaceutically acceptable salt thereof, wherein each X, Z, Q, and T may independently be defined as anywhere herein. In accordance with another embodiment, there is provided a use of a compound having a structure of Formula V

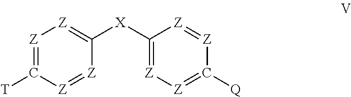

or a pharmaceutically acceptable salt thereof, wherein each X, Z, Q, and T may independently be defined as anywhere herein. In accordance with another embodiment, there is provided a use of a compound having a structure of Formula VI

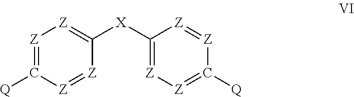

or a pharmaceutically acceptable salt thereof, wherein each X, Z, and Q may independently be defined as anywhere herein. In accordance with another embodiment, there is provided a use of a compound having a structure of Formula VII

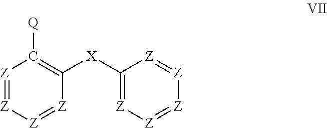

or a pharmaceutically acceptable salt thereof, wherein each X, Z, and Q may independently be defined as anywhere herein. In accordance with another embodiment, there is provided a use of a compound having a structure of Formula VIII

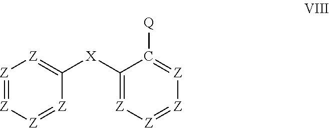

or a pharmaceutically acceptable salt thereof, wherein each X, Z, and Q may independently be defined as anywhere herein. In accordance with another embodiment, there is provided a use of a compound having a structure of Formula IX

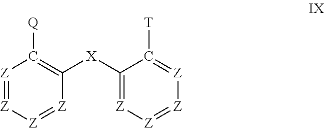

or a pharmaceutically acceptable salt thereof, wherein each X, Z, Q, and T may independently be defined as anywhere herein. In accordance with another embodiment, there is provided a use of a compound having a structure of Formula X

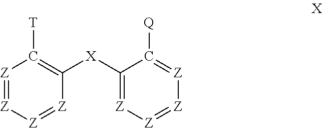

or a pharmaceutically acceptable salt thereof, wherein each X, Z, Q, and T may independently be defined as anywhere herein. In accordance with another embodiment, there is provided a use of a compound having a structure of Formula XI

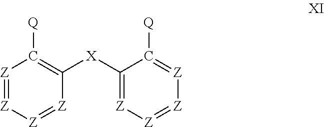

or a pharmaceutically acceptable salt thereof, wherein each X, Z, and Q may independently be defined as anywhere herein. In accordance with another embodiment, there is provided a use of a compound having a structure of Formula XII

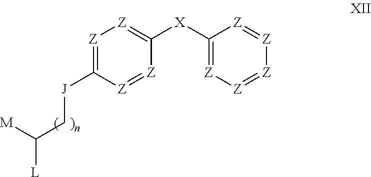

or a pharmaceutically acceptable salt thereof, wherein each X, Z, J, M, L, and n may independently be defined as anywhere herein. In accordance with another embodiment, there is provided a use of a compound having a structure of Formula XIII

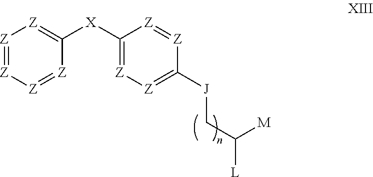

or a pharmaceutically acceptable salt thereof, wherein each X, Z, J, M, L, and n may independently be defined as anywhere herein. In accordance with another embodiment, there is provided a use of a compound having a structure of Formula XIV

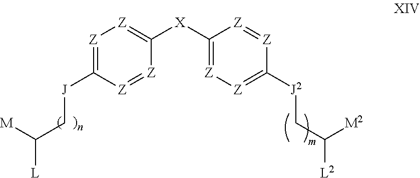

or a pharmaceutically acceptable salt thereof, wherein each X, Z, J, J², M, M², L, L², n, and m may independently be defined as anywhere herein. In accordance with another embodiment, there is provided a use of a compound having a structure of Formula XV

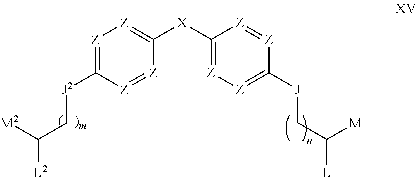

or a pharmaceutically acceptable salt thereof, wherein each X, Z, J, J², M, M², L, L²_{, n}, and m may independently be defined as anywhere herein. In accordance with another embodiment, there is provided a use of a compound having a structure of Formula XVI

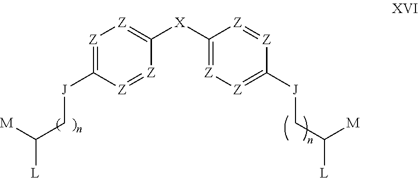

or a pharmaceutically acceptable salt thereof, wherein each X, Z, J, M, L, and n may independently be defined as anywhere herein. In accordance with another embodiment, there is provided a use of a compound having a structure of Formula XVII

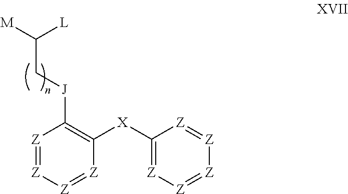

or a pharmaceutically acceptable salt thereof, wherein each X, Z, J, M, L, and n may independently be defined as anywhere herein. In accordance with another embodiment, there is provided a use of a compound having a structure of Formula XVIII

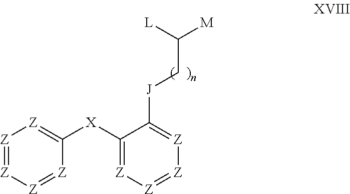

or a pharmaceutically acceptable salt thereof, wherein each X, Z, J, M, L, and n may independently be defined as anywhere herein. In accordance with another embodiment, there is provided a use of a compound having a structure of Formula XIX

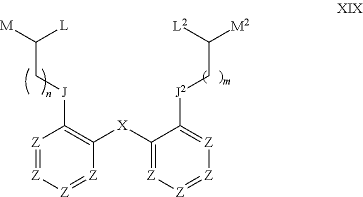

or a pharmaceutically acceptable salt thereof, wherein each X, Z, J, J², M, M², L, L², n, and m may independently be defined as anywhere herein. In accordance with another embodiment, there is provided a use of a compound having a structure of Formula XX

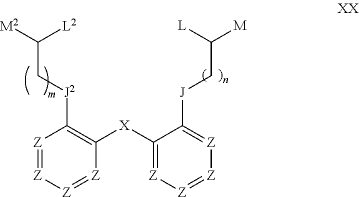

or a pharmaceutically acceptable salt thereof, wherein each X, Z, J, J², M, M², L, L², n, and m may independently be defined as anywhere herein. In accordance with another embodiment, there is provided a use of a compound having a structure of Formula XXI

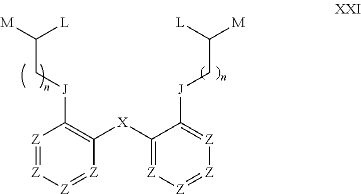

or a pharmaceutically acceptable salt thereof, wherein each X, Z, J, M, L, and n may independently be defined as anywhere herein.

Each J may independently be G¹, O, CH₂, CHG¹, CG¹₂, S, NH, NG¹, SO, SO₂, or NR. Each J may independently be G¹, O, CH₂, CHG¹, CG¹₂, S, NH, or NG¹. Each J may independently be O, S, NH, NG¹, SO, SO₂, or NR. Each J may independently be O, S, SO, or SO₂. Each J may independently be O, NH, NG¹, or NR. Each J may independently be S, NH, NG¹, SO, SO₂, or NR. Each J may independently be S, SO, or SO₂. Each J may independently be NH, NG¹, or NR. Each J may independently be G¹, CH₂, CHG¹, or CG¹₂. Each J may independently be O, CH₂, S, or NH. Each J may independently be O, CH₂, or NH. Each J may independently be O, or CH₂. Each J may independently be G¹, O, CHG¹, or NH. Each J may independently be G¹, O, or CHG¹. Each J may independently be G¹, or O. Each J may independently be O, or S. Each J may independently be G′. Each J may independently be CH₂. Each J may be CHG¹. Each J may be CG¹₂. Each J may be NR. Each J may be SO₂. Each J may be SO. Each J may be NG¹. Each J may be NH. Each J may be S. Each J may be O.

Each M may independently be H, Cl, Br, CH₂Cl, CHCl₂, CCl₃, CH₂Br, CHBr₂, CBr₃, or Each M may independently be Cl, Br, CH₂Cl, CHCl₂, CCl₃, CH₂Br, CHBr₂, or CBr₃. Each M may independently be Cl, CH₂Cl, CHCl₂, or CCl₃. Each M may independently be Br, CH₂Br, CHBr₂, or CBr₃. Each M may independently be Cl, or Br. Each M may independently be CH₂Cl, or CH₂Br. Each M may independently be CHCl₂, or CHBr₂. Each M may independently be CCl₃, or CBr₃. Each M may independently be CH₂Cl, CHCl₂, or CCl₃. Each M may independently be CH₂Br, CHBr₂, or CBr₃. Each M may independently be Cl, CH₂Cl, or CHCl₂. Each M may independently be Br, CH₂Br, or CHBr₂. Each M may independently be CH₂Cl, or CHCl₂. Each M may independently be CH₂Br, or CHBr₂. Each M may independently be Cl, or CCl₃. Each M may independently be Br, or CBr₃. Each M may be H. Each M may be Cl. Each M may be Br. Each M may be CHCl₂. Each M may be CCl₃. Each M may be CH₂Br. Each M may be CHBr₂. Each M may be CBr₃. Each M may be C≡CH. Each M may be CH₂Cl.

Each L may independently be H or A-D. Each L may be H. Each L may be A-D.

Each A may independently be O, S, NH, NG¹, N⁺H₂, or N⁺HG¹. Each A may independently be O, NH, or N⁺H₂. Each A may independently be O, S, NH, or N⁺H₂. Each A may independently be O, S, or NH. Each A may independently be O, or NH. Each A may independently be O, or S. Each A may be S. Each A may be NH. Each A may be NG¹. Each A may be N⁺H₂. Each A may be N⁺HG¹. Each A may be O.

Each D may independently be H, G¹, R,

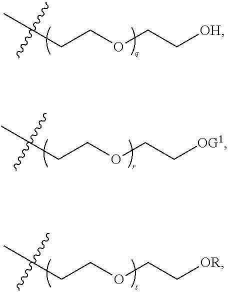

or a moiety selected from TABLE 1. Each D may independently be H, G¹, or R. Each D may independently be H, or R. Each D may independently be G¹, or R. Each D may independently be H, or G′. Each D may independently be

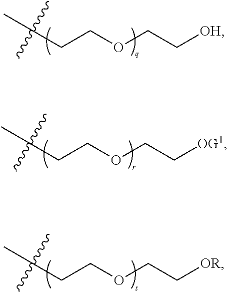

or a moiety selected from TABLE 1. Each D may independently be

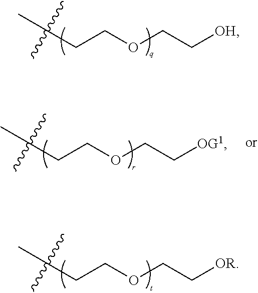

Each D may independently be

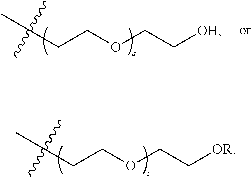

, Each D may independently be

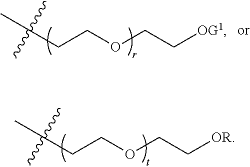

. Each D may independently be

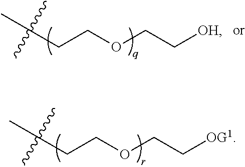

Each D may independently be

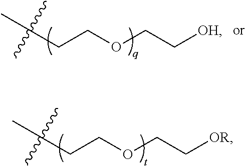

or a moiety selected from TABLE 1. Each D may independently be

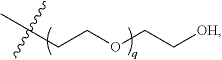

or a moiety selected from TABLE 1. Each D may be H. Each D may be G¹. Each D may be R. Each D may be

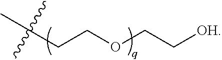

### Each D may be

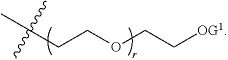

### Each D may be

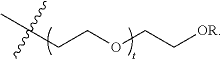

Each D may be a moiety selected from TABLE 1.

Each J² may independently be G¹, O, CH₂, CHG¹, CG¹₂, S, NH, NG¹, SO, SO₂, or NR. Each J² may independently be G¹, O, CH₂, CHG¹, CG¹₂, S, NH, or NG¹. Each J² may independently be O, S, NH, NG¹, SO, SO₂, or NR. Each J² may independently be O, S, SO, or SO₂. Each J² may independently be O, NH, NG¹, or NR. Each J² may independently be S, NH, NG¹, SO, SO₂, or NR. Each J² may independently be S, SO, or SO₂. Each J² may independently be NH, NG¹, or NR. Each J² may independently be G¹, CH₂, CHG¹, or CG¹₂. Each J² may independently be O, CH₂, S, or NH. Each J² may independently be O, CH₂, or NH. Each J² may independently be O, or CH₂. Each J² may independently be G¹, O, CHG¹, or NH. Each J² may independently be G¹, O, or CHG¹. Each J² may independently be G¹, or O. Each J² may independently be O, or S. Each J² may independently be G¹. Each J² may independently be CH₂. Each J² may be CHG¹. Each J² may be CG¹₂. Each J² may be NR. Each J² may be SO₂. Each J² may be SO. Each J² may be NG¹. Each J² may be NH. Each J² may be S. Each J² may be O.

Each M² may independently be H, CH₃, Cl, Br, CH₂Cl, CHCl₂, CCl₃, CH₂Br, CHBr₂, CBr₃, CH₂OH, CH₂OJ″, G¹, CH₂OG¹, CH₂OR, CH₂OG¹OG¹′, G¹OG¹, G¹OG¹′OG¹″, CH₂SG¹, CH₂NH₂, CH₂NHG¹, CH₂NG¹₂, or C≡CH. Each M² may independently be H, CH₃, CH₂Cl, CH₂Br, CH₂OJ′″, CH₂OG, CH₂OGOG¹, GOG¹, GOG′OG″, CH₂SG, CH₂NH₂, CH₂NHG, or CH₂NG₂. Each M² may independently be H, CH₃, CH₂Cl, CH₂Br, CH₂Or, CH₂OG, or CH₂OGOG′. Each M² may independently be CH₂Cl, CH₂Br, CH₂OH, CH₂OCH₃, CH₂O(isopropyl), or CH₂OC₂H₄₀C₄H₉. Each M² may independently be H, CH₃, CH₃OCH₃, CH₃OCH₂CH₃, CH₂Cl, or CH₂Br. Each M² may independently be CH₃, CH₃OCH₂CH₃, CH₂Cl, CH₂Br, CH₂OH, CH₂OCH₃, or CH₂O(isopropyl). Each M² may independently be CH₃, CH₂Cl, CH₂Br, CH₂OH, CH₃OCH₂CH₃, or CH₂OCH₃. Each M² may independently be CH₃, CH₂Cl, CH₂Br, CH₂OH, or CH₂OCH₃. Each M² may independently be CH₃, CH₂OH, CH₂OCH₃, or CH₂OCH₂CH₃. Each M² may independently be CH₂Cl, or CH₂Br. Each M² may independently be H, Cl, Br, CH₂Cl, CHCl₂, CCl₃, CH₂Br, CHBr₂, CBr₃, or C≡CH. Each M may independently be Cl, Br, CH₂Cl, CHCl₂, CCl₃, CH₂Br, CHBr₂, or CBr₃. Each M² M² i may independently be Cl, CH₂Cl, CHCl₂, or CCl₃. Each M² may independently be Br, CH₂Br, CHBr₂, or CBr₃. Each M² may independently be Cl, or Br. Each M² may independently be CH₂Cl, or CH₂Br. Each M² may independently be CHCl₂, or CHBr₂. Each M² may independently be CCl₃, or CBr₃. Each M² may independently be CH₂Cl, CHCl₂, or CCl₃. Each M² may independently be CH₂Br, CHBr₂, or CBr₃. Each M² may independently be Cl, CH₂Cl, or CHCl₂. Each M² may independently be Br, CH₂Br, or CHBr₂. Each M² may independently be CH₂Cl, or CHCl₂. Each M² may independently be CH₂Br, or CHBr₂. Each M² may independently be Cl, or CCl₃. Each M² may independently be Br, or CBr₃. Each M² may be H. Each M² may be CH₃. Each M² may be Cl. Each M² may be Br. Each M² may be CH₂Cl. Each M² may be CHCl₂. Each M² may be CCl₃. Each M² may be CH₂Br. Each M² may be CHBr₂. Each M² may be CBr₃. Each M² may be CH₂OH. Each M² may be CH₂OJ″. Each M² may be G¹. Each M² may be CH₂OG¹. Each M² may be CH₂OR. Each M² may be CH₂OG¹OG¹′. Each M² may be G¹OG¹′. Each M² may be G¹OG¹′OG¹″. Each M² may be CH₂SG¹. Each M² may be CH₂NH₂. Each M² may be CH₂NHG¹. Each M² may be CH₂NG¹₂. Each M² may be C═CH.

Each L² may independently be H or A²-D². Each L² may be H. Each L² may be A²-D².

Each A² may independently be O, S, SO, SO₂, NH, NG¹, N⁺H₂, or N⁺HG¹. Each A² may independently be O, S, SO, or SO₂. Each A² may independently be O, NH, NG¹, N⁺H₂, or N⁺HG¹. Each A² may independently be S, SO, SO₂, NH, NG¹, N⁺H₂, or N⁺HG¹. Each A² may independently be O, S, SO, SO₂, NH, or N⁺H₂. Each A² may independently be S, SO, or SO₂. Each A² may independently be NH, NG¹, N⁺H₂, or N⁺HG¹. Each A² may independently be NH, or N⁺H₂. Each A² may independently be O, S, NH, NG¹, NH⁺H₂, or N⁺HG¹. Each A² may independently be O, NH, or N⁺H₂. Each A² may independently be O, S, NH, or N⁺H₂. Each A² may independently be O, S, or NH. Each A² may independently be O, or NH. Each A² may independently be O, or S. Each A² may be S. Each A² may be SO. Each A² may be SO₂. Each A² may be NH. Each A² may be NG¹. Each A² may be N⁺H₂. Each A² may be N⁺HG¹. Each A² may be O.

Each D² may independently be H, G¹, R,

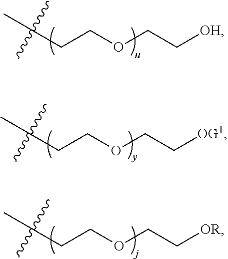

, or a moiety selected from TABLE 1. Each D² may independently be H, G¹, or R. Each D² may independently be H, or R. Each D² may independently be G¹, or R. Each D² may independently be H, or G′. Each D² may independently be

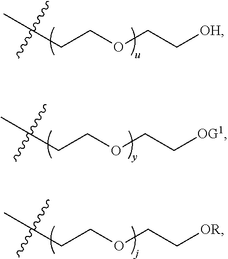

or a moiety selected from TABLE 1. Each D² may independently be

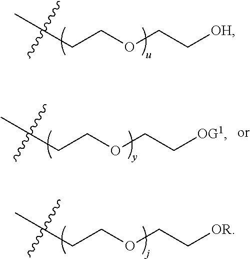

Each D² may independently be

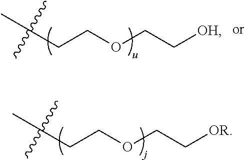

Each D² may independently be

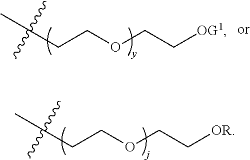

Each D² may independently be

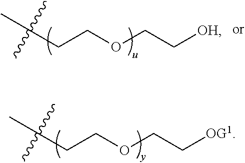

Each D² may independently be

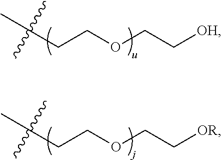

or a moiety selected from TABLE 1. Each D² may independently be

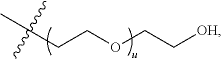

or a moiety selected from TABLE 1. Each D² may be H. Each D² may be G′. Each D² may be R. Each D² may be

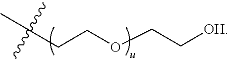

### Each D² may be Each D² may be

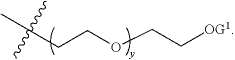

Each D² may be a moiety selected from TABLE 1.

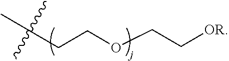

Each Q may independently be

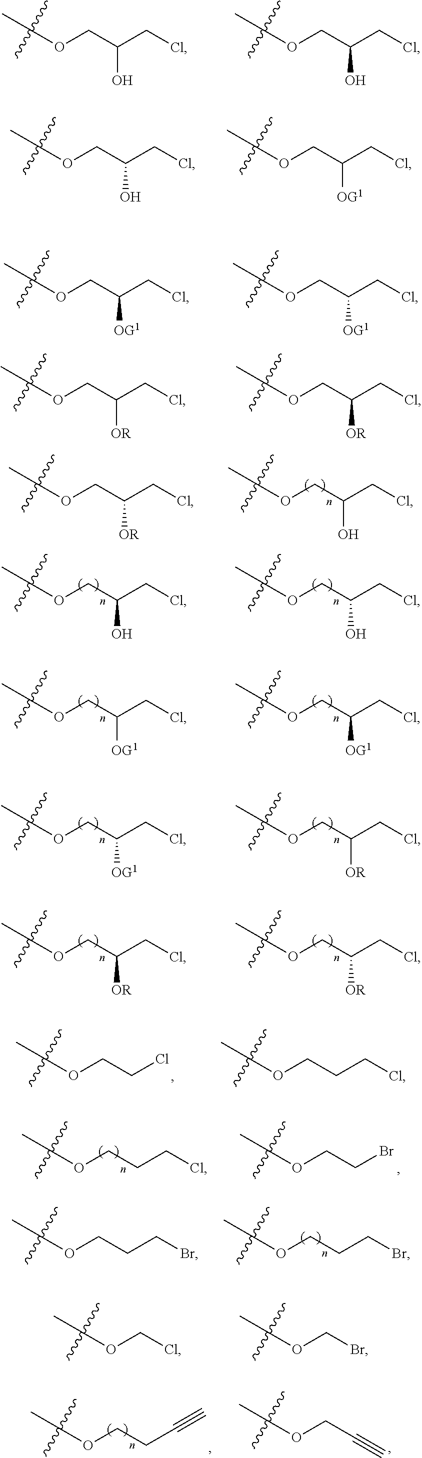

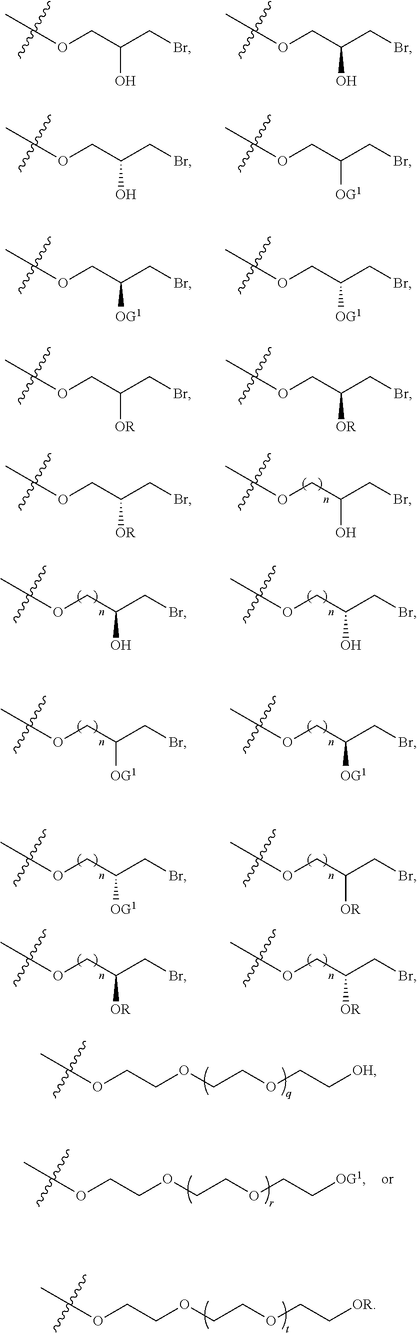

Each Q may independently be

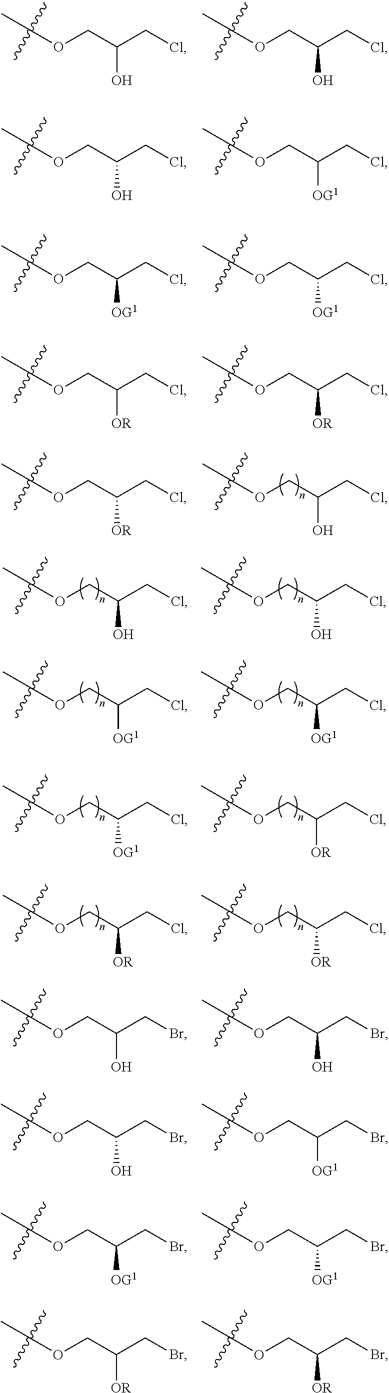

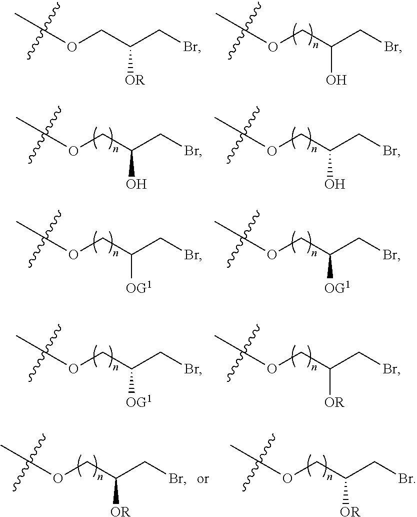

Each Q may independently be

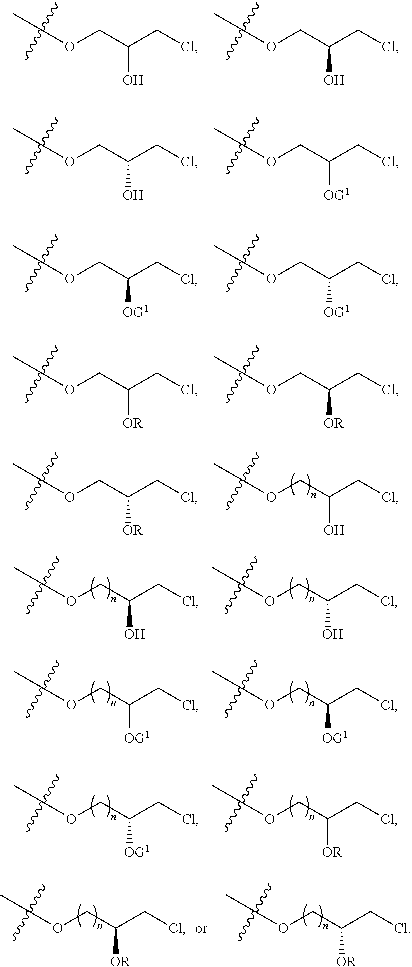

Each Q may independently be

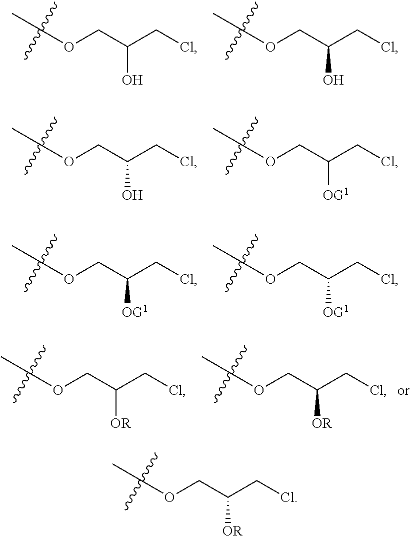

Each Q may independently be

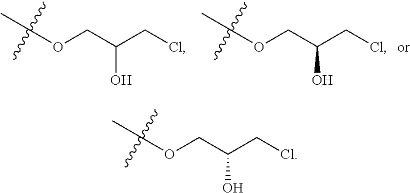 [SMILES: *OCC(O)CCl ; *OC[C@H](O)CCl ; *OC[C@@H](O)CCl]

Each Q may independently be

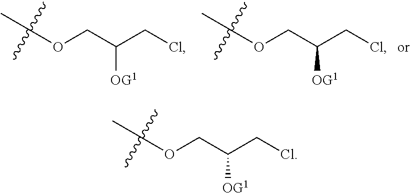

Each Q may independently be

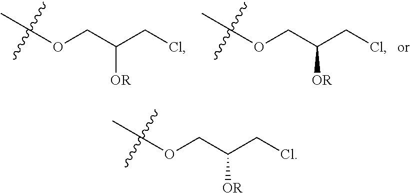

Each Q may independently be

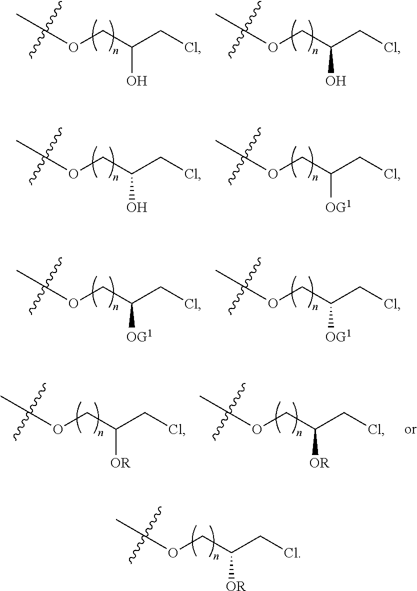

Each Q may independently be

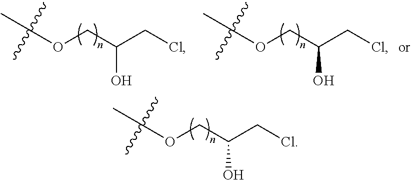

Each Q may independently be

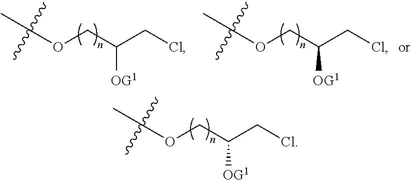

Each Q may independently be

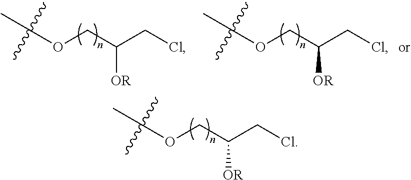

Each Q may independently be

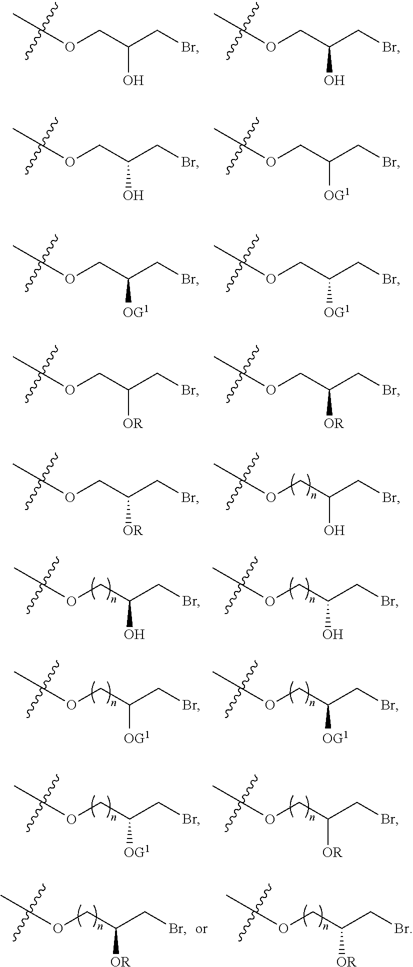

Each Q may independently be

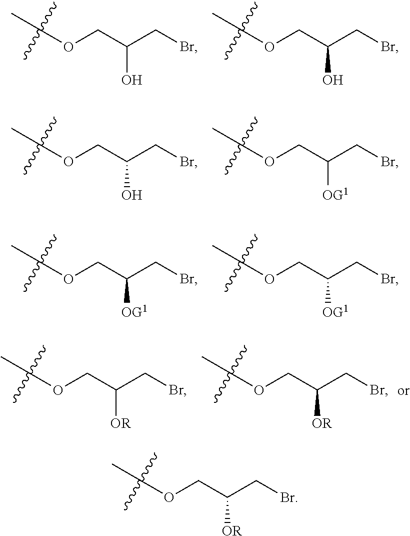

Each Q may independently be

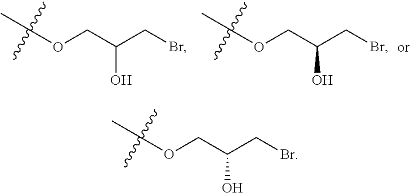 [SMILES: *OCC(O)CBr ; *OC[C@H](O)CBr ; *OC[C@@H](O)CBr]

Each Q may independently be

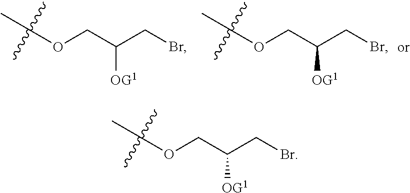

Each Q may independently be

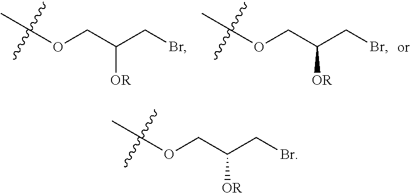

Each Q may independently be

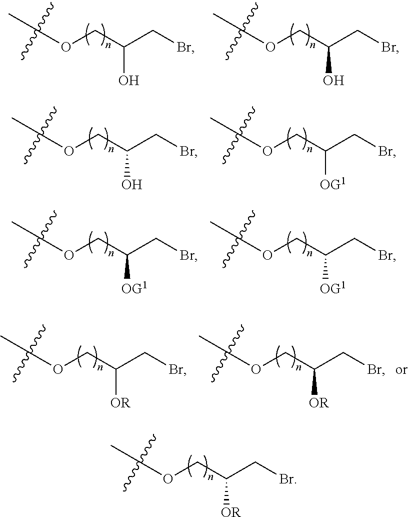

Each Q may independently be

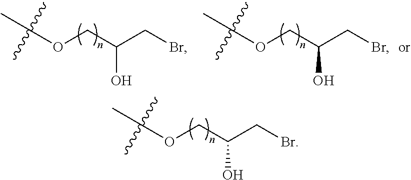

Each Q may independently be

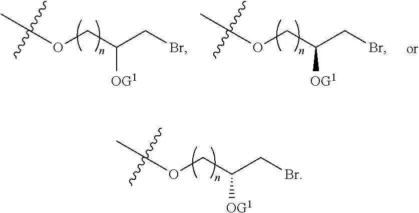

Each Q may independently be

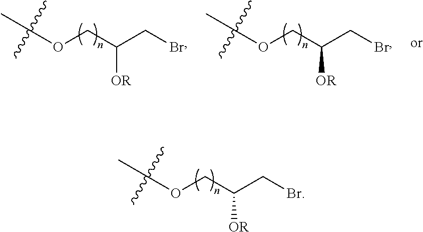

Each Q may independently be

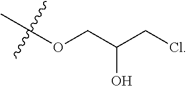 [SMILES: *OCC(O)CCl]

Each Q may independently be

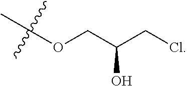 [SMILES: *OC[C@H](O)CCl]

Each Q may independently be

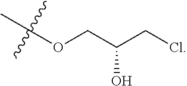 [SMILES: *OC[C@@H](O)CCl]

Each Q may independently be

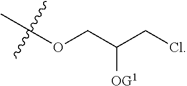

Each Q may independently be

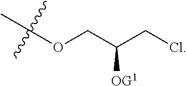

Each Q may independently be

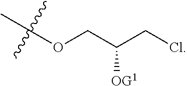

Each Q may independently be

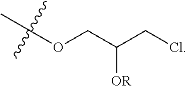

Each Q may independently be

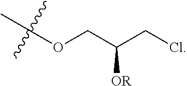

Each Q may independently be

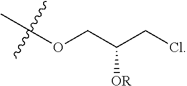

Each Q may independently be

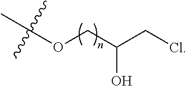

Each Q may independently be

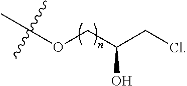

Each Q may independently be

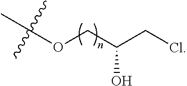

Each Q may independently be

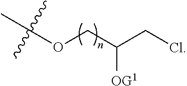

Each Q may independently be

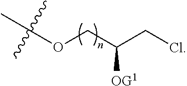

Each Q may independently be

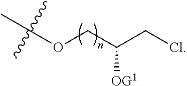

Each Q may independently be

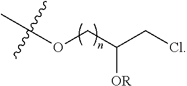

Each Q may independently be

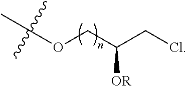

Each Q may independently be

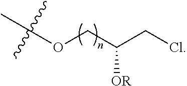

Each Q may independently be

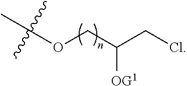

Each Q may independently be

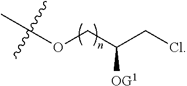

Each Q may independently be

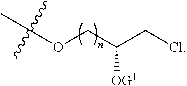

Each Q may independently be

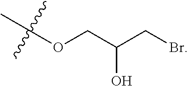 [SMILES: *OCC(O)CBr]

Each Q may independently be

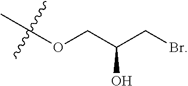 [SMILES: *OC[C@H](O)CBr]

Each Q may independently be

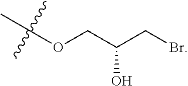 [SMILES: *OC[C@@H](O)CBr]

Each Q may independently be

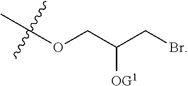

Each Q may independently be

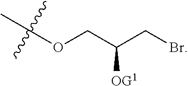

Each Q may independently be

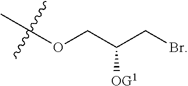

Each Q may independently be

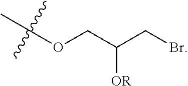

Each Q may independently be

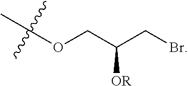

Each Q may independently be

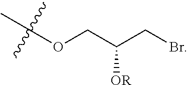

Each Q may independently be

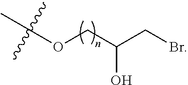

Each Q may independently be

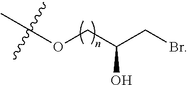

Each Q may independently be

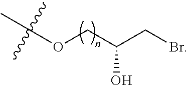

Each Q may independently be

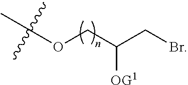

Each Q may independently be

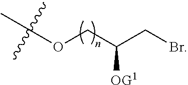

Each Q may independently be

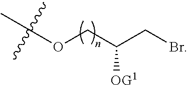

Each Q may independently be

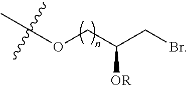

Each Q may independently be

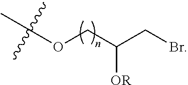

Each Q may independently be

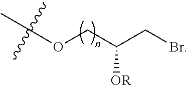

Each Q may independently be

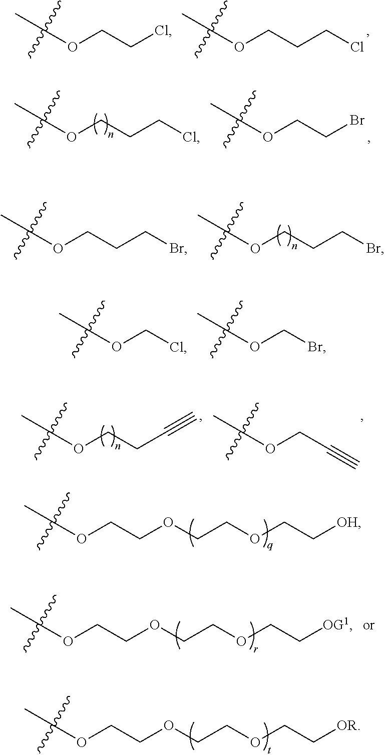

Each Q may independently be

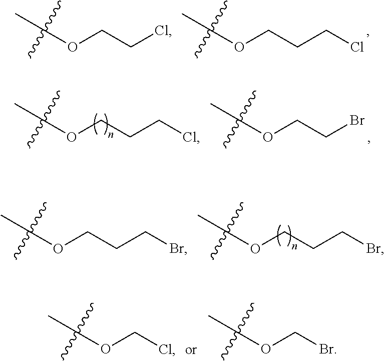

Each Q may independently be

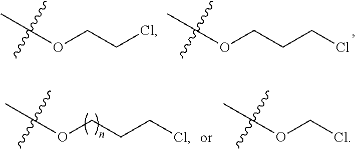

Each Q may independently be

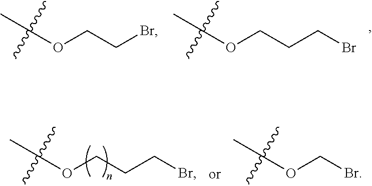

Each Q may independently be

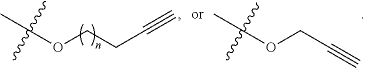

Each Q may independently

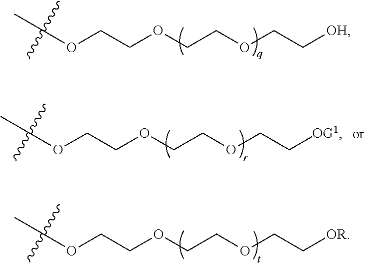

### Each Q may be

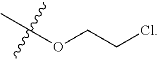 [SMILES: *OCCCl]

### Each Q may be

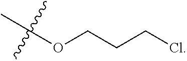 [SMILES: *OCCCCl]

Each Q may independently be

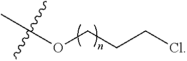

### Each Q may be

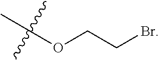 [SMILES: *OCCBr]

### Each Q may be

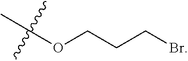 [SMILES: *OCCCBr]

Each Q may independently be

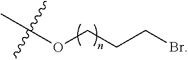

### Each Q may be

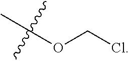 [SMILES: *OCCl]

### Each Q may be

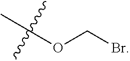 [SMILES: *OCBr]

Each Q may independently be

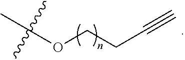

### Each Q may be

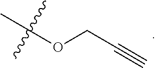 [SMILES: *OCC#C]

Each Q may independently be

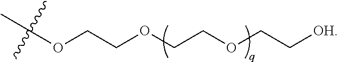

Each Q may independently be

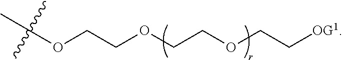

Each Q may independently be

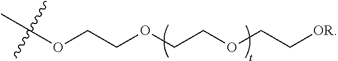

Each T may independently be

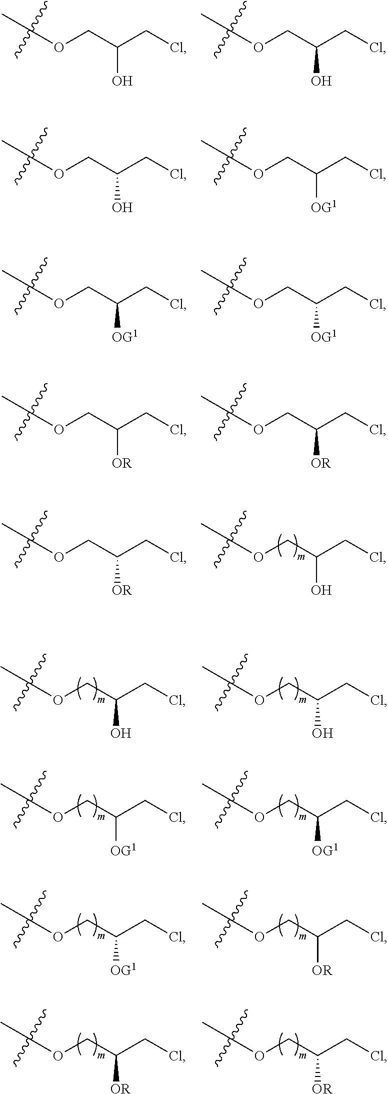

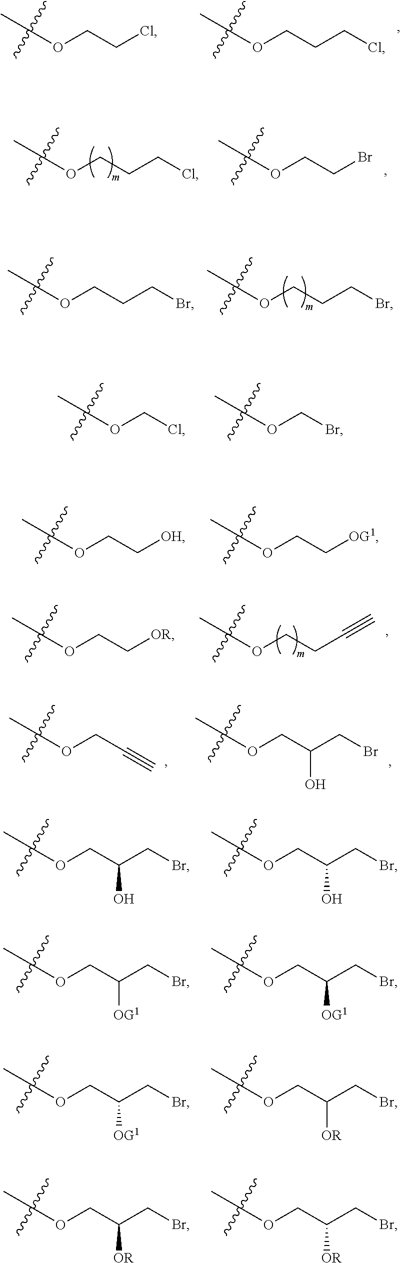

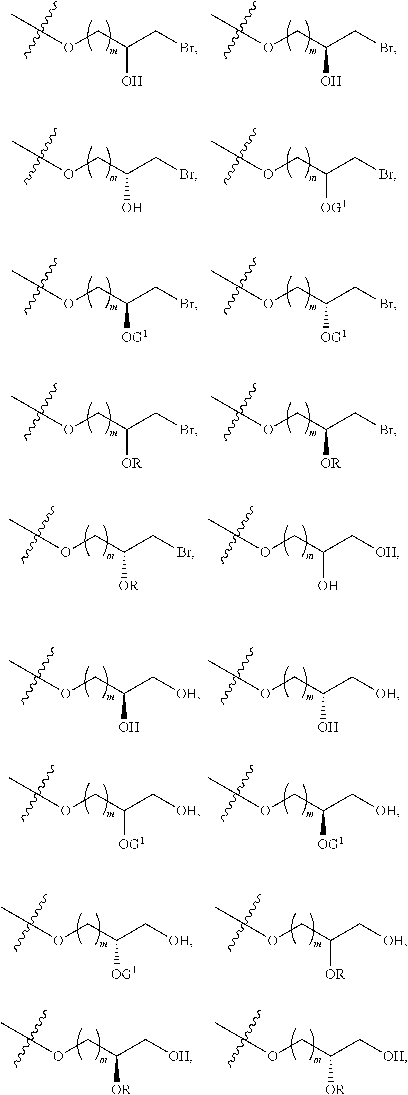

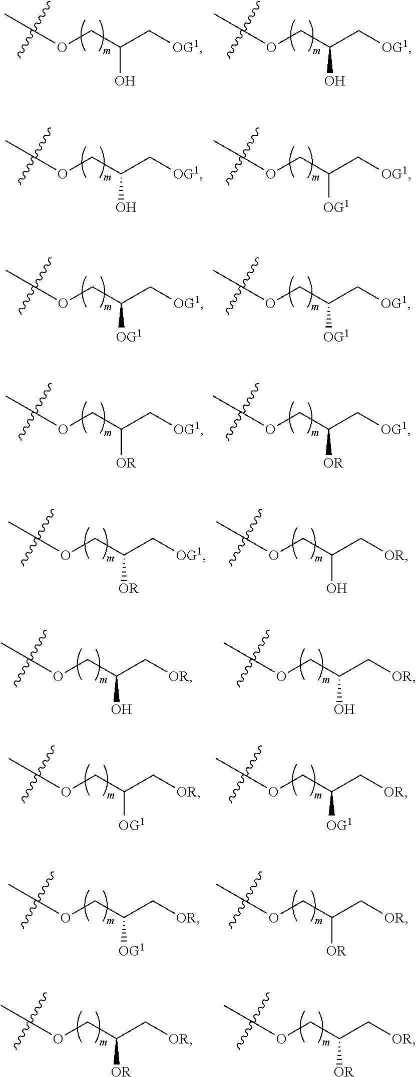

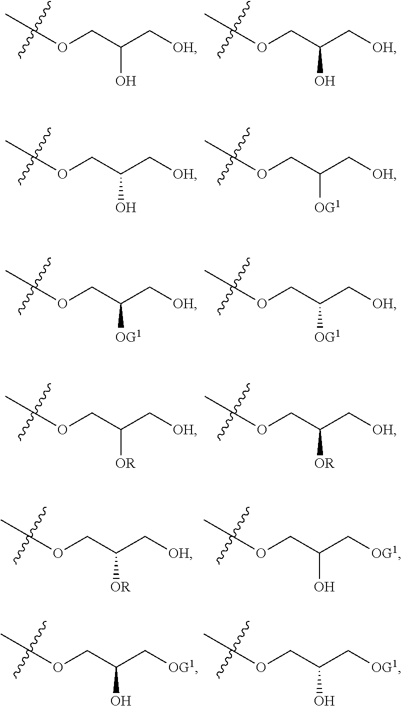

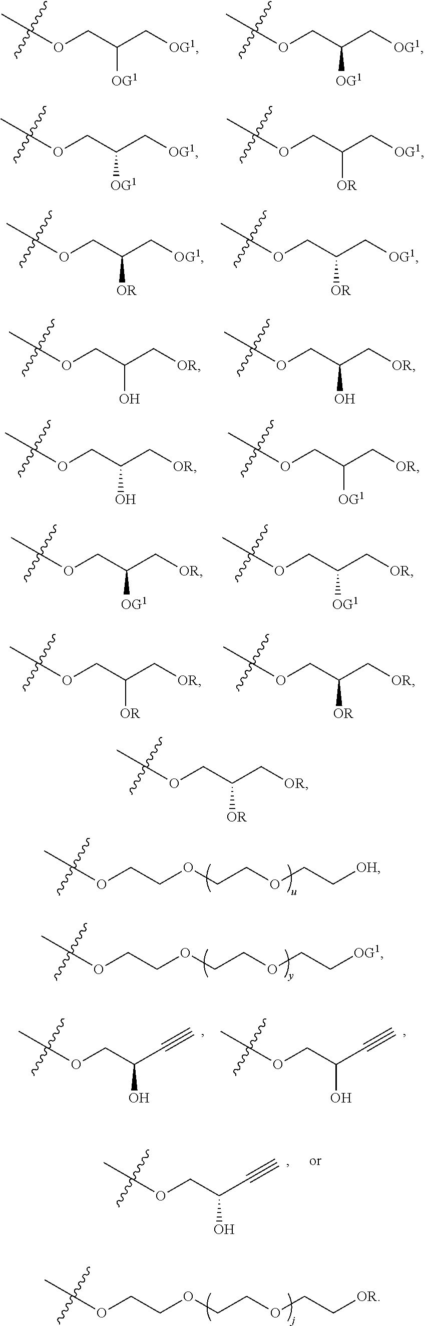

Each T may independently be

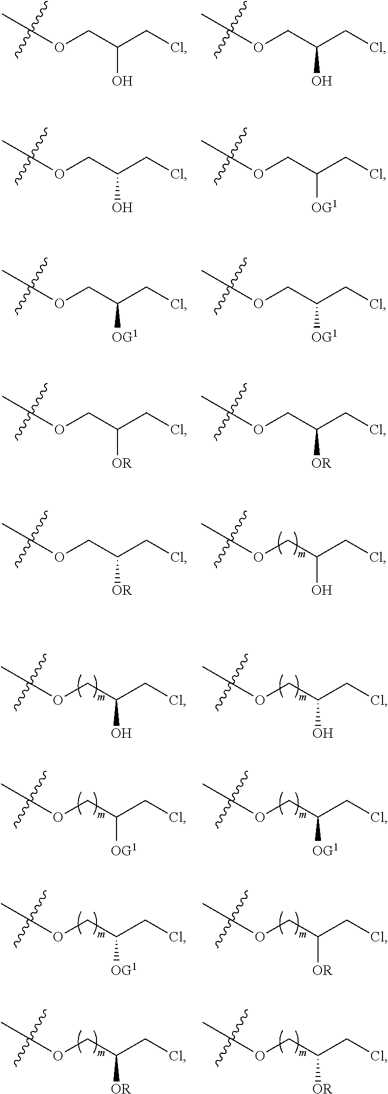

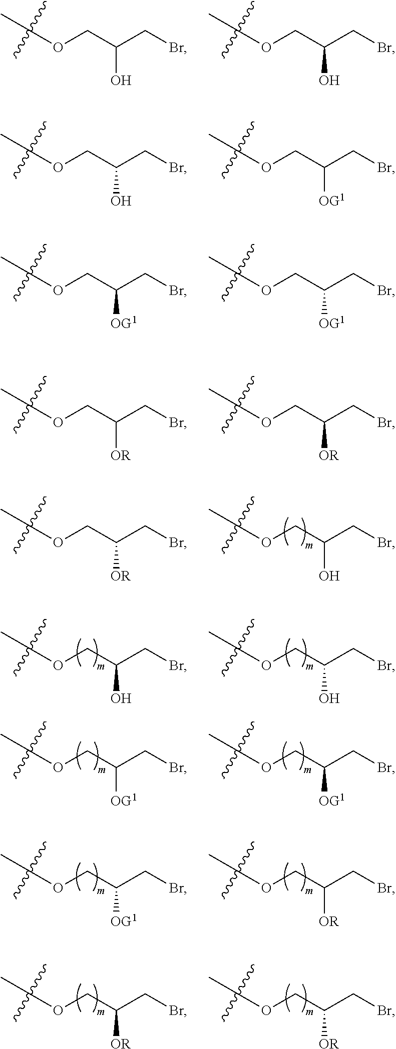

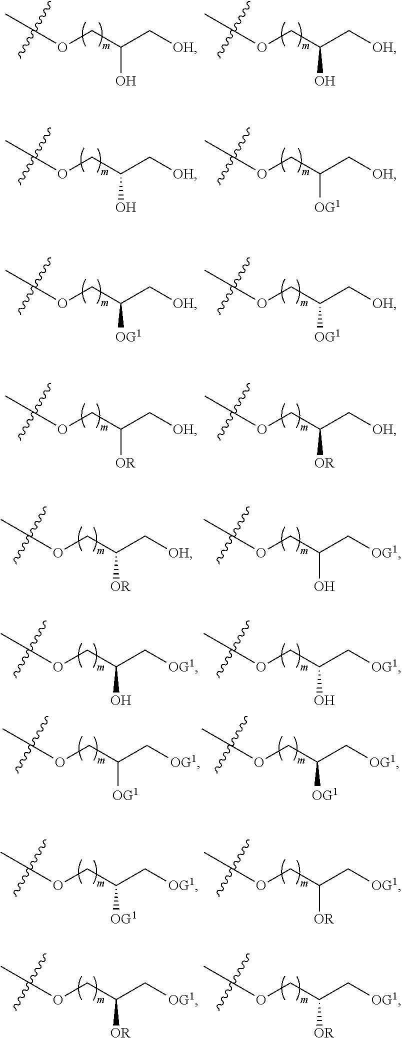

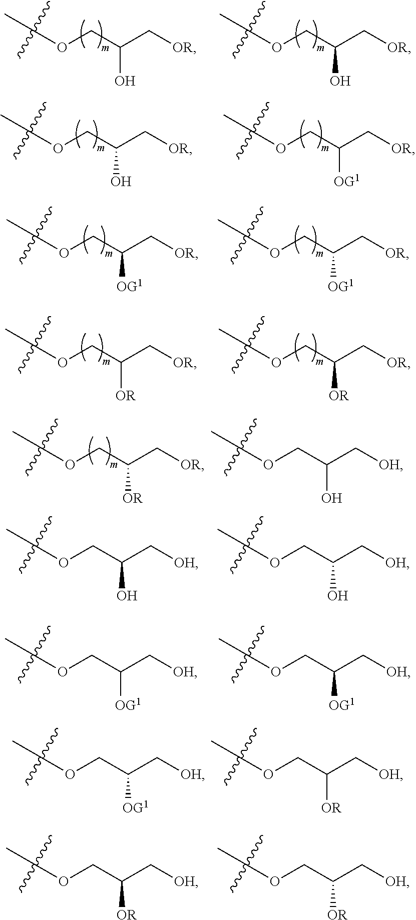

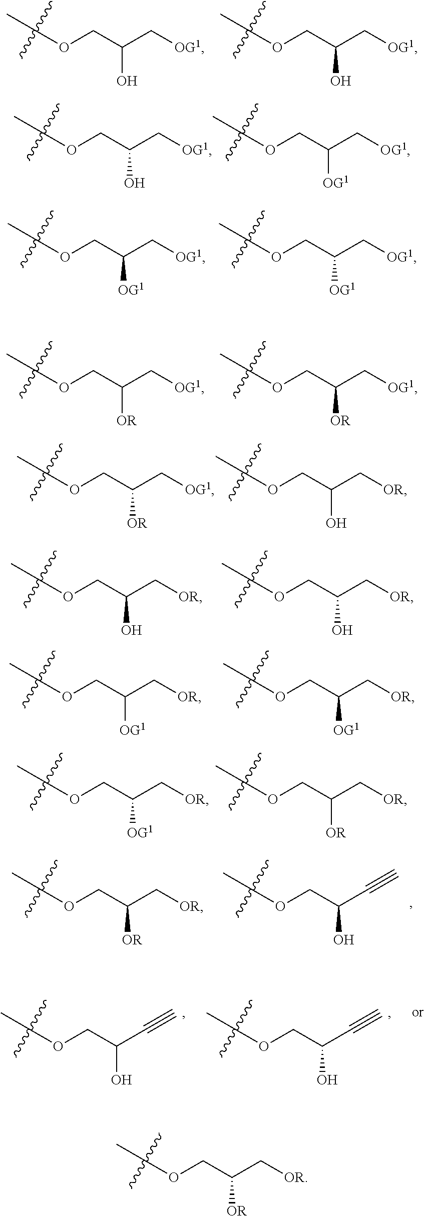

Each T may independently be

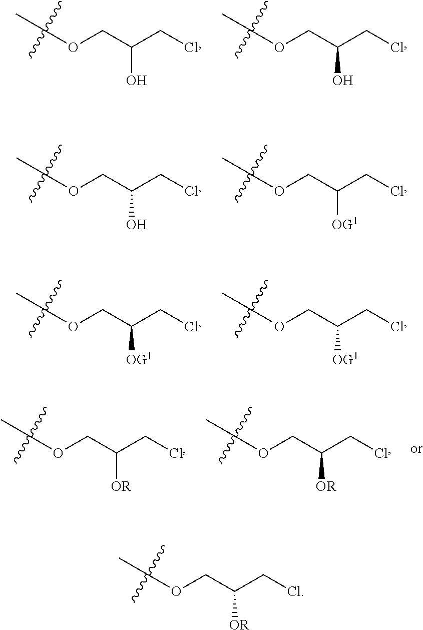

Each T may independently be

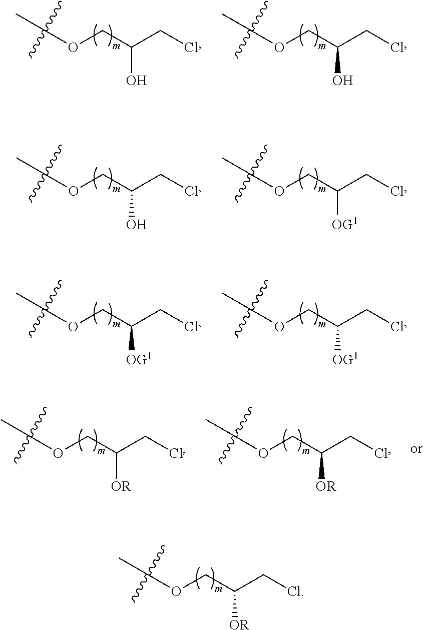

Each T may independently be

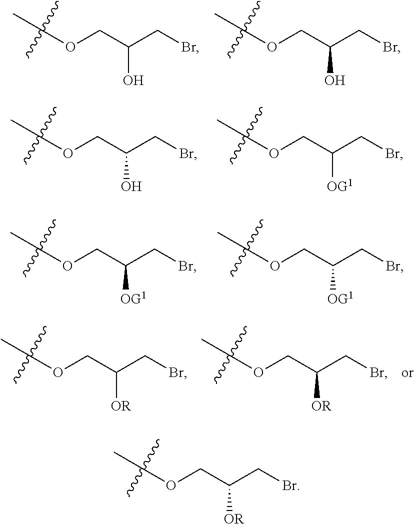

Each T may independently be

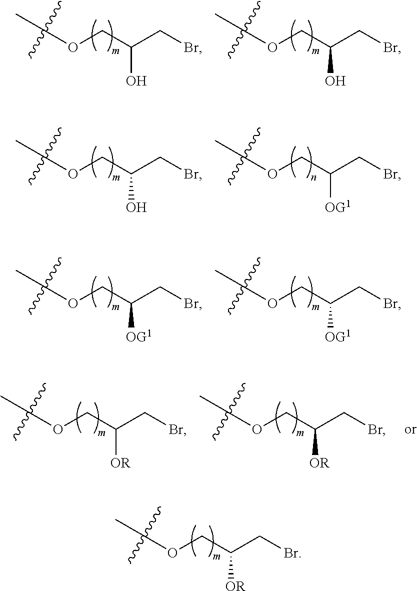

Each T may independently be

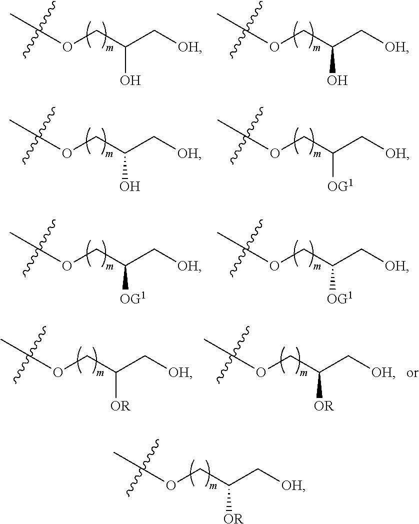

Each T may independently be

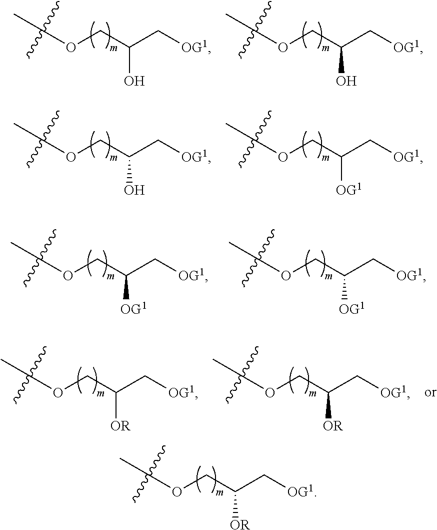

Each T may independently be

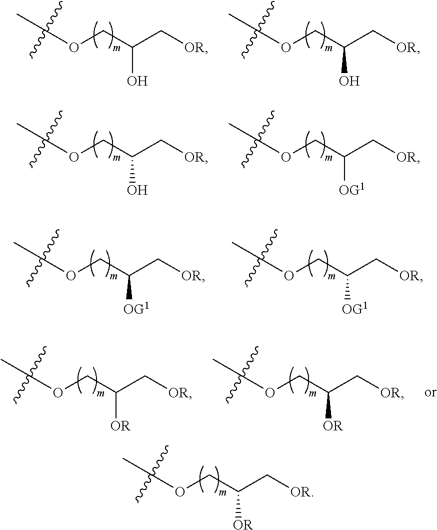

Each T may independently be

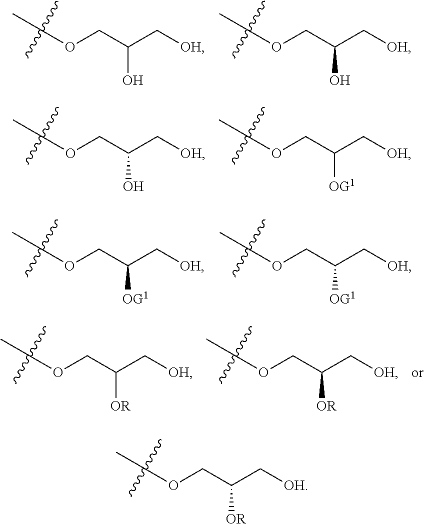

Each T may independently be

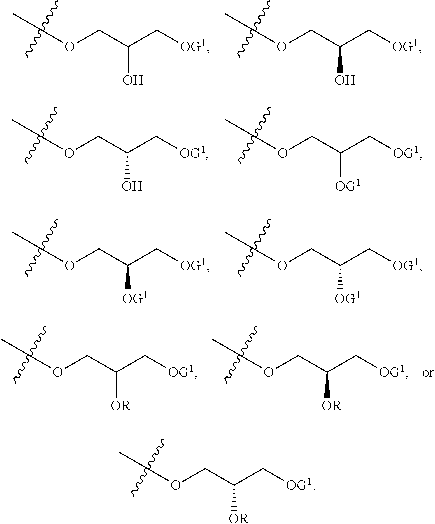

Each T may independently be

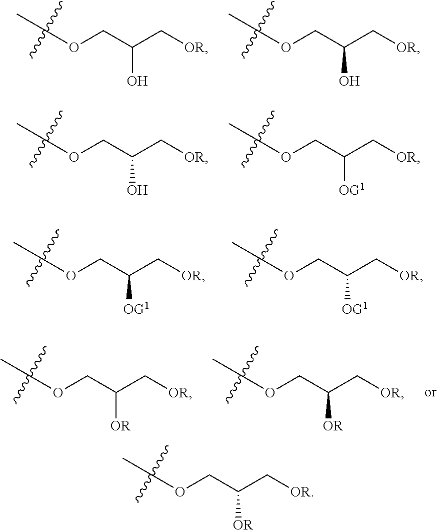

Each T may independently be

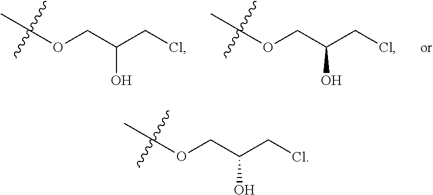 [SMILES: *OCC(O)CCl ; *OC[C@H](O)CCl ; *OC[C@@H](O)CCl]

Each T may independently be

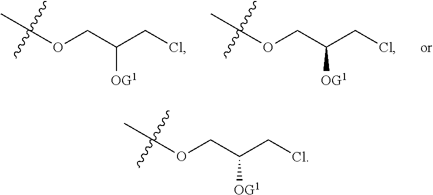

Each T may independently be

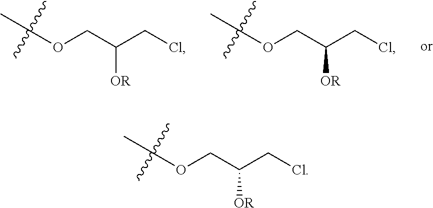

Each T may independently be

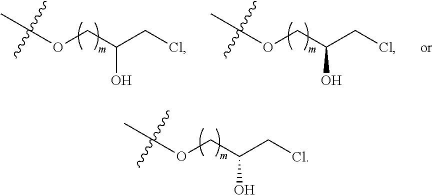

Each T may independently be

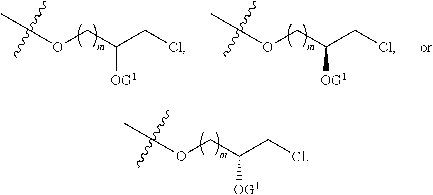

Each T may independently be

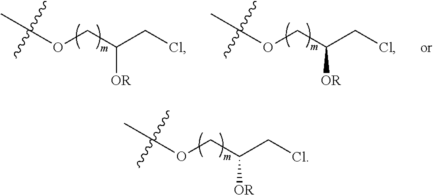

Each T may independently be

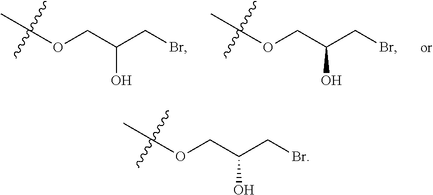 [SMILES: *OCC(O)CBr ; *OC[C@H](O)CBr ; *OC[C@@H](O)CBr]

Each T may independently be

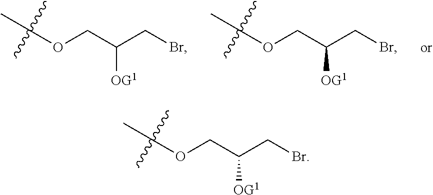

Each T may independently be

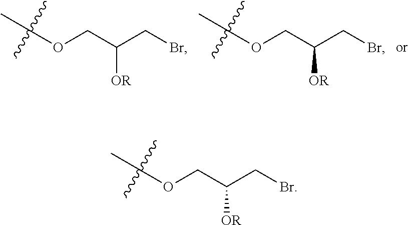

Each T may independently be

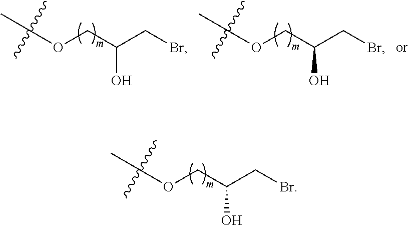

Each T may independently be

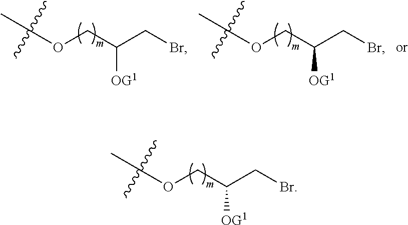

Each T may independently be

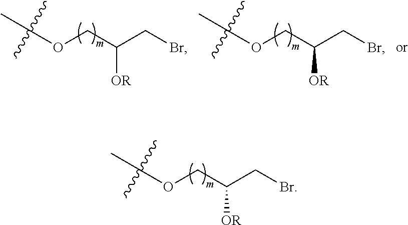

Each T may independently be

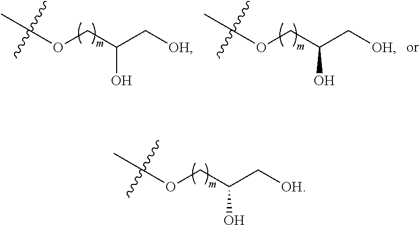

Each T may independently be

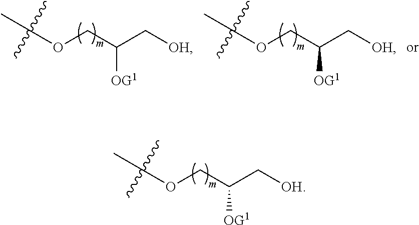

Each T may independently be

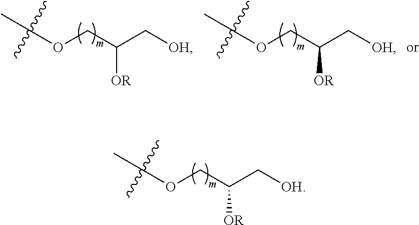

Each T may independently be

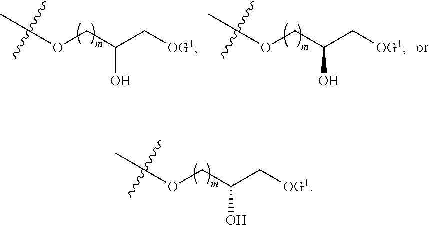

Each T may independently be

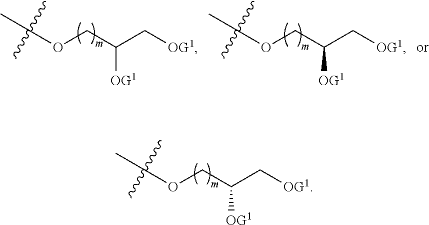

Each T may independently be

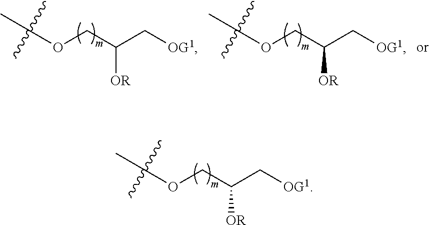

Each T may independently be

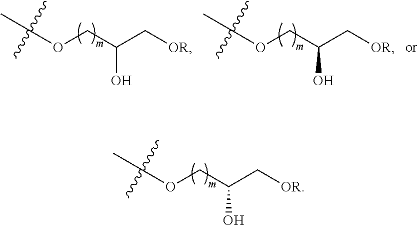

Each T may independently be

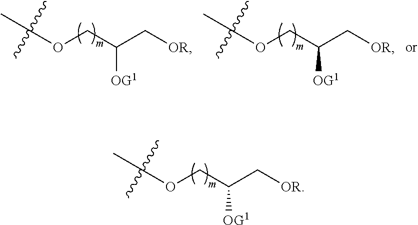

Each T may independently be

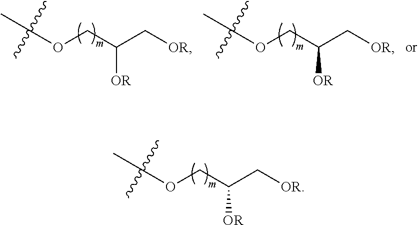

Each T may independently be

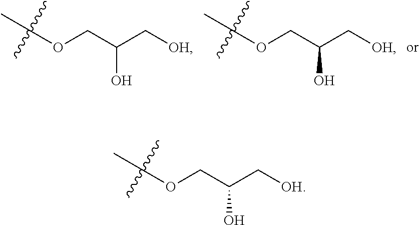 [SMILES: *OCC(O)CO ; *OC[C@H](O)CO ; "or" ; *OC[C@@H](O)CO]

Each T may independently be

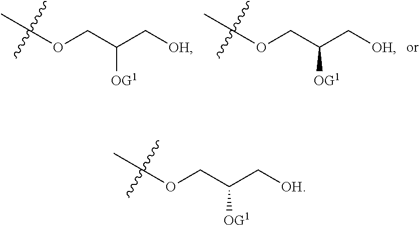

Each T may independently be

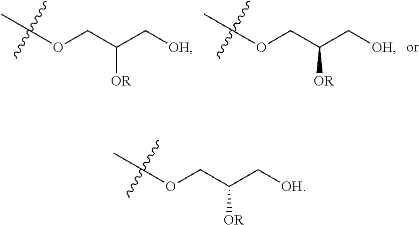

Each T may independently be

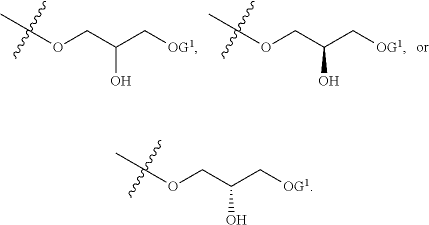

Each T may independently be

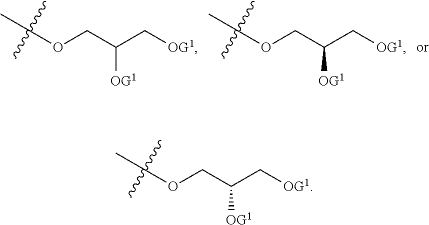

Each T may independently be

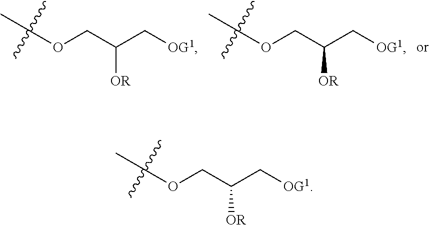

Each T may independently be

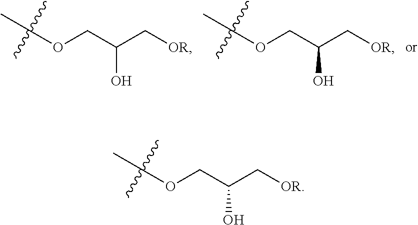

Each T may independently be

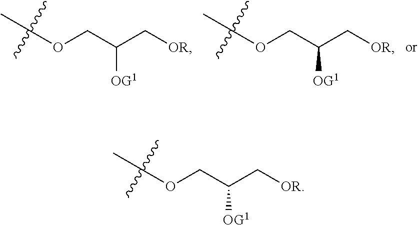

Each T may independently be

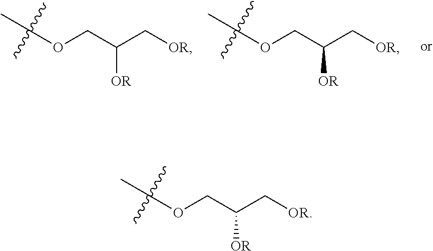

Each T may independently be

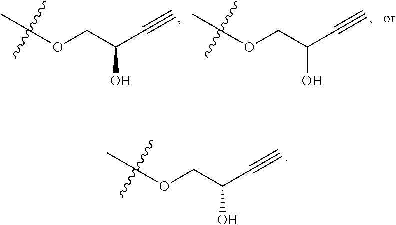

Each T may independently be

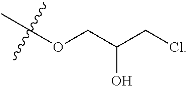 [SMILES: *OCC(O)CCl]

Each T may independently be

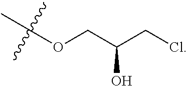 [SMILES: *OC[C@H](O)CCl]

Each T may independently be

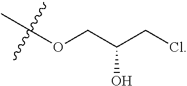 [SMILES: *OC[C@@H](O)CCl]

Each T may independently be

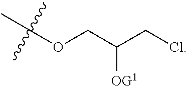

Each T may independently be

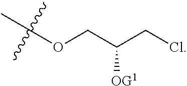

Each T may independently be

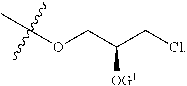

Each T may independently be

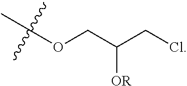

Each T may independently be

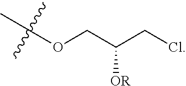

Each T may independently be

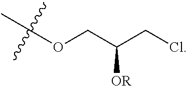

Each T may independently be

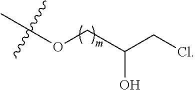

Each T may independently be

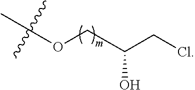

Each T may independently be

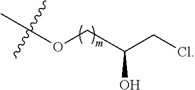

Each T may independently be

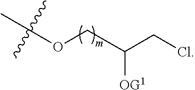

Each T may independently be

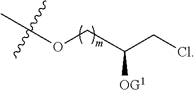

Each T may independently be

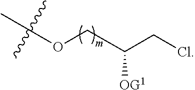

Each T may independently be

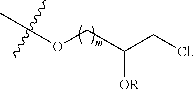

Each T may independently be

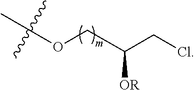

Each T may independently be

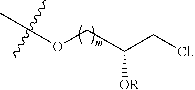

Each T may independently be

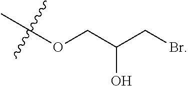 [SMILES: *OCC(O)CBr]

Each T may independently be

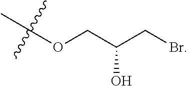 [SMILES: *OC[C@@H](O)CBr]

Each T may independently be

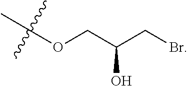 [SMILES: *OC[C@H](O)CBr]

Each T may independently be

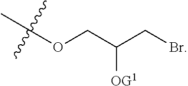

Each T may independently be

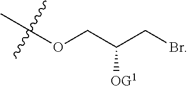

Each T may independently be

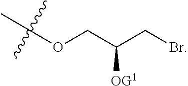

Each T may independently be

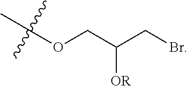

Each T may independently be

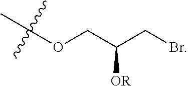

Each T may independently be

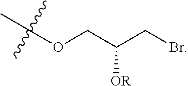

Each T may independently be

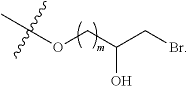

Each T may independently be

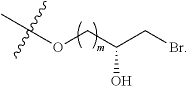

Each T may independently be

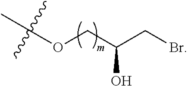

Each T may independently be

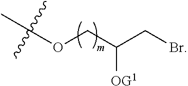

Each T may independently be

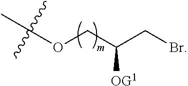

Each T may independently be

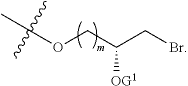

Each T may independently be

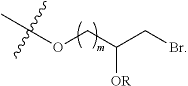

Each T may independently be

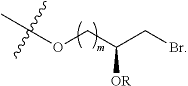

Each T may independently be

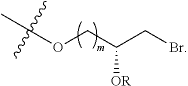

Each T may independently be

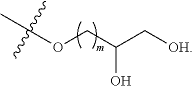

Each T may independently be

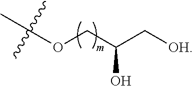

Each T may independently be

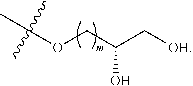

Each T may independently be

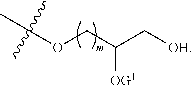

Each T may independently be

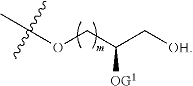

Each T may independently be

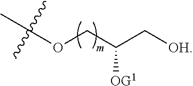

Each T may independently be

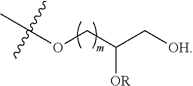

Each T may independently be

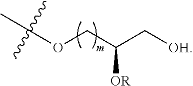

Each T may independently be

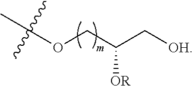

Each T may independently be

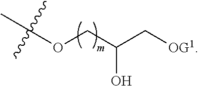

Each T may independently be

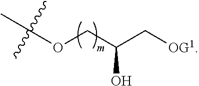

Each T may independently be

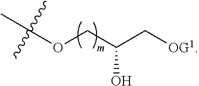

Each T may independently be

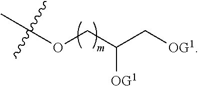

Each T may independently be

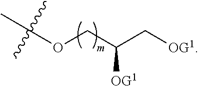

Each T may independently be

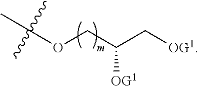

Each T may independently be

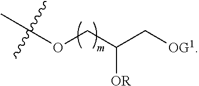

Each T may independently be

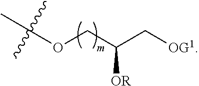

Each T may independently be

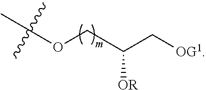

Each T may independently be

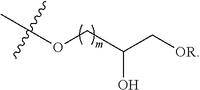

Each T may independently be

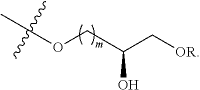

Each T may independently be

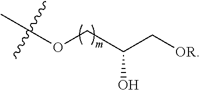

Each T may independently be

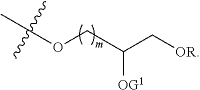

Each T may independently be

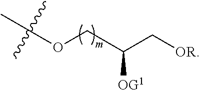

Each T may independently be

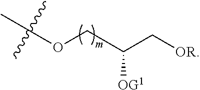

Each T may independently be

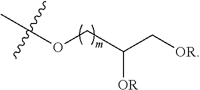

Each T may independently be

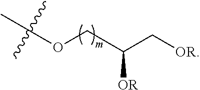

Each T may independently be

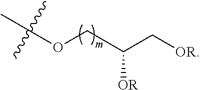

Each T may independently be

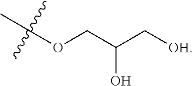 [SMILES: *OCC(O)CO]

Each T may independently be

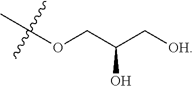 [SMILES: *OC[C@H](O)CO]

Each T may independently be

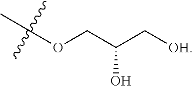 [SMILES: *OC[C@@H](O)CO]

Each T may independently be

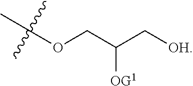

Each T may independently be

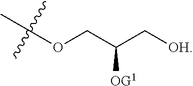

Each T may independently be

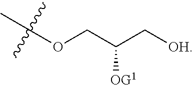

Each T may independently be

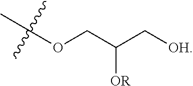

Each T may independently be

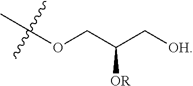

Each T may independently be

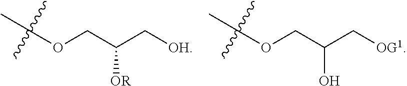

Each T may independently be

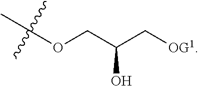

Each T may independently be

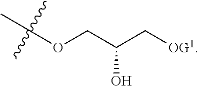

Each T may independently be

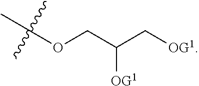

Each T may independently be

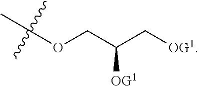

Each T may independently be

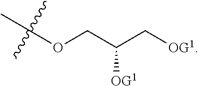

Each T may independently be

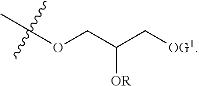

. Each T may independently be

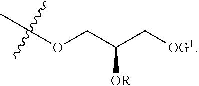

Each T may independently be

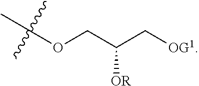

Each T may independently be

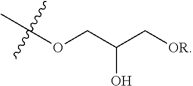

Each T may independently be

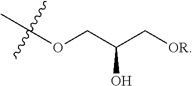

Each T may independently be

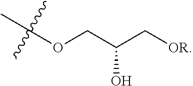

Each T may independently be

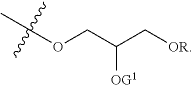

Each T may independently be

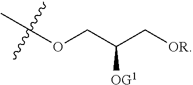

Each T may independently be

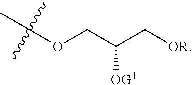

Each T may independently be

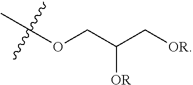

Each T may independently be

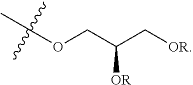

Each T may independently be

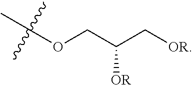

Each T may independently be

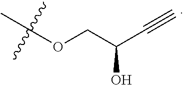 [SMILES: *OC[C@H](O)C#C ; "."]

Each T may independently be

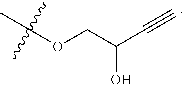 [SMILES: *OCC(O)C#C ; "."]

Each T may independently be

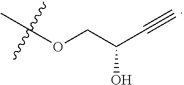 [SMILES: *OC[C@@H](O)C#C ; "."]

Each T may independently be

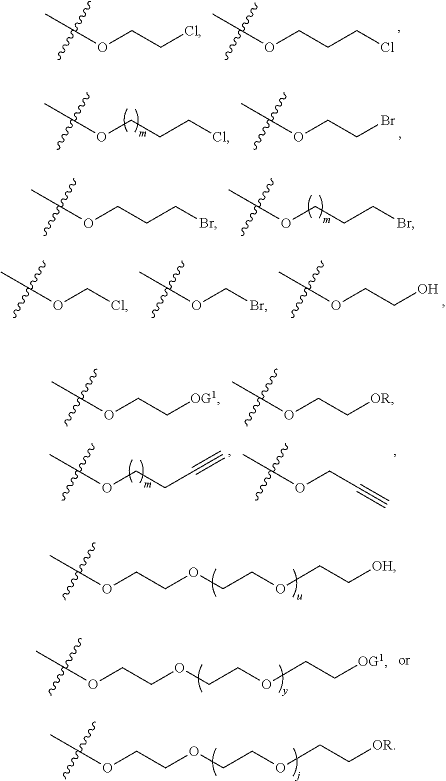

Each T may independently be

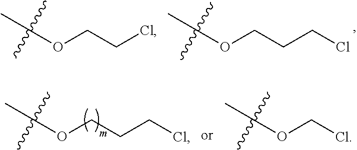

Each T may independently be

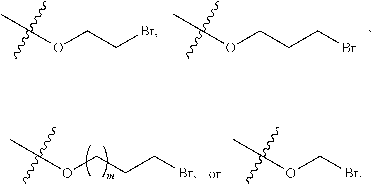

Each T may independently be

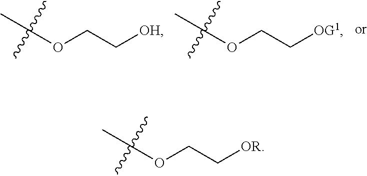

Each T may independently be

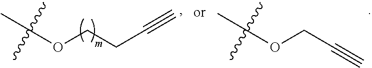

Each T may independently be

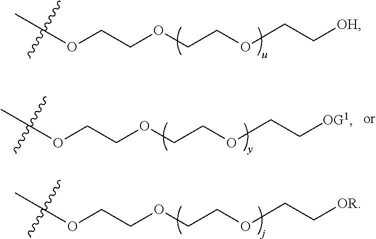

Each T may independently be

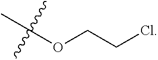 [SMILES: *OCCCl]

Each T may independently be

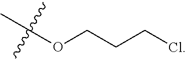 [SMILES: *OCCCCl]

Each T may independently be

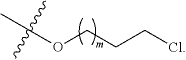

Each T may independently be

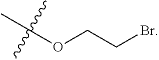 [SMILES: *OCCBr]

Each T may independently be

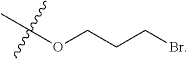 [SMILES: *OCCCBr]

Each T may independently be

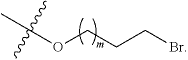

Each T may independently be

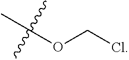 [SMILES: *OCCl]

Each T may independently be

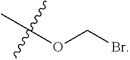 [SMILES: *OCBr]

Each T may independently be

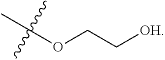 [SMILES: *OCCO]

Each T may independently be

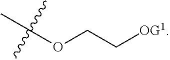

Each T may independently be

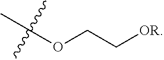

Each T may independently be

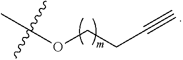

Each T may independently be

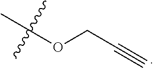 [SMILES: *OCC#C]

Each T may independently be

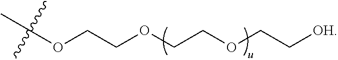

Each T may independently be

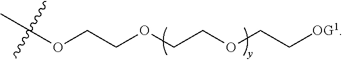

Each T may independently be

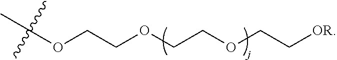

Each q may independently be 0, 1, 2, 3, 4, 5, 6 or 7. Each q may independently be 0 to 1, 0 to 2, 0 to 3, 0 to 4, 0 to 5, 0 to 6, or 0 to 7. Each q may independently be 1 to 2, 1 to 3, 1 to 4, 1 to 5, 1 to 6, or 1 to 7. Each q may independently be 2 to 3, 2 to 4, 2 to 5, 2 to 6, or 2 to 7. Each q may independently be 3 to 4, 3 to 5, 3 to 6, or 3 to 7. Each q may be 0. Each q may be 1. Each q may be 2. Each q may be 3. Each q may be 4. Each q may be 5. Each q may be 6. Each q may be 7.

Each r may independently be 0, 1, 2, 3, 4, 5, 6 or 7. Each r may independently be 0 to 1, 0 to 2, 0 to 3, 0 to 4, 0 to 5, 0 to 6, 0 to 7. Each r may independently be 1 to 2, 1 to 3, 1 to 4, 1 to 5, 1 to 6, or 1 to 7. Each r may independently be 2 to 3, 2 to 4, 2 to 5, 2 to 6, or 2 to 7. Each r may independently be 3 to 4, 3 to 5, 3 to 6, or 3 to 7. Each r may be 0. Each r may be 1. Each r may be 2. Each r may be 3. Each r may be 4. Each r may be 5. Each r may be 6. Each r may be 7.

Each t may independently be 0, 1, 2, 3, 4, 5, 6 or 7. Each t may independently be 0 to 1, 0 to 2, 0 to 3, 0 to 4, 0 to 5, 0 to 6, or 0 to 7. Each t may independently be 1 to 2, 1 to 3, 1 to 4, 1 to 5, 1 to 6, or 1 to 7. Each t may independently be 2 to 3, 2 to 4, 2 to 5, 2 to 6, or 2 to 7. Each t may independently be 3 to 4, 3 to 5, 3 to 6, or 3 to 7. Each t may be 0. Each t may be 1. Each t may be 2. Each t may be 3. Each t may be 4. Each t may be 5. Each t may be 6. Each t may be 7.

Each n may independently be 0, 1, 2, 3, 4, 5, 6, 7 or 8. Each n may independently be 0 to 1, 0 to 2, 0 to 3, 0 to 4, 0 to 5, 0 to 6, 0 to 7, or 0 to 8. Each n may independently be 1 to 2, 1 to 3, 1 to 4, 1 to 5, 1 to 6, 1 to 7, or 1 to 8. Each n may independently be 2 to 3, 2 to 4, 2 to 5, 2 to 6, 2 to 7, or 2 to 8. Each n may independently be 3 to 4, 3 to 5, 3 to 6, 3 to 7, or 3 to 8. Each n may be 0. Each n may be 1. Each n may be 2. Each n may be 3. Each n may be 4. Each n may be 5. Each n may be 6. Each n may be 7. Each n may be 8.

Each u may independently be 0, 1, 2, 3, 4, 5, 6 or 7. Each u may independently be 0 to 1, 0 to 2, 0 to 3, 0 to 4, 0 to 5, 0 to 6, 0 to 7. Each u may independently be 1 to 2, 1 to 3, 1 to 4, 1 to 5, 1 to 6, or 1 to 7. Each u may independently be 2 to 3, 2 to 4, 2 to 5, 2 to 6, or 2 to 7. Each u may independently be 3 to 4, 3 to 5, 3 to 6, or 3 to 7. Each u may be 0. Each u may be 1. Each u may be 2. Each u may be 3. Each u may be 4. Each u may be 5. Each u may be 6. Each u may be 7.

Each y may independently be 0, 1, 2, 3, 4, 5, 6 or 7. Each y may independently be 0 to 1, 0 to 2, 0 to 3, 0 to 4, 0 to 5, 0 to 6, or 0 to 7. Each y may independently be 1 to 2, 1 to 3, 1 to 4, 1 to 5, 1 to 6, or 1 to 7. Each y may independently be 2 to 3, 2 to 4, 2 to 5, 2 to 6, or 2 to 7. Each y may independently be 3 to 4, 3 to 5, 3 to 6, or 3 to 7. Each y may be 0. Each y may be 1. Each y may be 2. Each y may be 3. Each y may be 4. Each y may be 5. Each y may be 6. Each y may be 7.

Each j may independently be 0, 1, 2, 3, 4, 5, 6 or 7. Each j may independently be 0 to 1, 0 to 2, 0 to 3, 0 to 4, 0 to 5, 0 to 6, or 0 to 7. Each j may independently be 1 to 2, 1 to 3, 1 to 4, 1 to 5, 1 to 6, or 1 to 7. Each j may independently be 2 to 3, 2 to 4, 2 to 5, 2 to 6, or 2 to 7. Each j may independently be 3 to 4, 3 to 5, 3 to 6, or 3 to 7. Each j may be 0. Each j may be 1. Each j may be 2. Each j may be 3. Each j may be 4. Each j may be 5. Each j may be 6. Each j may be 7.

Each m may independently be 0, 1, 2, 3, 4, 5, 6, 7 or 8. Each m may independently be 0 to 1, 0 to 2, 0 to 3, 0 to 4, 0 to 5, 0 to 6, 0 to 7, or 0 to 8. Each m may independently be 1 to 2, 1 to 3, 1 to 4, 1 to 5, 1 to 6, 1 to 7, or 1 to 8. Each m may independently be 2 to 3, 2 to 4, 2 to 5, 2 to 6, 2 to 7, or 2 to 8. Each m may independently be 3 to 4, 3 to 5, 3 to 6, 3 to 7, or 3 to 8. Each m may be 0. Each m may be 1. Each m may be 2. Each m may be 3. Each m may be 4. Each m may be 5. Each m may be 6. Each m may be 7. Each m may be 8.

At least one Z of one aromatic ring may independently be C-Q, at least one Z of the other aromatic ring may independently be C-T, CF, CCl, CBr, CI, COH, CG¹, COG¹, CNH₂, CNG¹₂, COSO₃H, COPO₃H₂, CSG¹, CSOG¹, or CSO₂G¹, and each remaining Z may independently be C-T, N, CH, CF, CCl, CBr, CI, COH, CG¹, COG¹, CNH₂, CNHG¹, CNG¹₂, COSO₃H, COPO₃H₂, CSG¹, CSOG¹, or CSO₂G′. At least one Z of one aromatic ring may independently be C-Q, at least one Z of the other aromatic ring may independently be C-T, and each remaining Z may independently be N, CH, CF, CCl, CBr, CI, CG¹, or COH. At least one Z of one aromatic ring may independently be C-Q, at least one Z of the other aromatic ring may independently be COH, and each remaining Z may independently be N, CH, CF, CCl, CBr, C¹, CG¹, or COH. At least one Z of one aromatic ring may independently be C-Q, at least one Z of the other aromatic ring may independently be C-T, and each remaining Z may independently be N, CH, CF, CCl, CBr, CI, or COH. At least one Z of one aromatic ring may independently be C-Q, at least one Z of the other aromatic ring may independently be COH, and each remaining Z may independently be N, CH, CF, CCl, CBr, CI, or COH. At least one Z of one aromatic ring may independently be C-Q, at least one Z of the other aromatic ring may independently be C-T, and each remaining Z may independently be CH. At least one Z of one aromatic ring may independently be C-Q, at least one Z of the other aromatic ring may independently be COH, and each remaining Z may independently be CH. Each remaining Z may independently be N, CH, CF, CCl, CBr, CI, COH, CCH₃, CNH₂, COSO₃H, or COPO₃H₂. Each remaining Z may independently be N, CH, CF, CCl, CBr, CI, COH, CNH₂, COSO₃H, or COPO₃H₂. Each remaining Z may independently be N, CH, CF, CCl, CBr, CI, or COH. Each remaining Z may independently be CH, CF, CCl, CBr, or CI. Each remaining Z may independently be CH, CCl, or CBr. Each remaining Z may be CH.

Each of J″ and J′″ may independently be a moiety selected from TABLE 1. Each of J″, and J′″ may independently be an amino acid based moiety or a polyethylene glycol based moiety selected from TABLE 1. Alternatively, each of J″, and J′″ may independently an amino acid based moiety selected from TABLE 1. Each J″, and J′″ may be

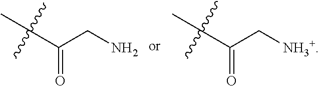

Each G¹ G¹′ and G¹″ may independently be linear or branched, or aromatic cyclic or non-aromatic cyclic, substituted or unsubstituted, saturated or unsaturated C₁-C₁₀ alkyl. Each G¹, G¹′ and G¹″ may independently be a branched, linear, or non-aromatic cyclic, substituted or unsubstituted, saturated or unsaturated C₁-C₁₀ alkyl. Each G¹, G¹, and G¹′ may independently be a branched, linear, or non-aromatic cyclic, substituted or saturated or unsaturated C₁-C₁₀ alkyl. Each G¹, G¹′ and G¹″ may independently be a branched, unbranched, or aromatic cyclic or non-aromatic cyclic, substituted or unsubstituted, saturated or unsaturated C₁-C₉ alkyl. Each G¹, G^{1′} and G¹″ may independently be a branched, unbranched, or aromatic cyclic or non-aromatic cyclic, substituted or unsubstituted, saturated or unsaturated C₁-C₈ alkyl. Each G¹, G¹, and G¹″ may independently be a branched, unbranched, or aromatic cyclic or non-aromatic cyclic, substituted or unsubstituted, saturated or unsaturated C₁-C₇ alkyl. Each G¹, G¹′ and G¹″ may independently be a branched, unbranched, or aromatic cyclic or non-aromatic cyclic, substituted or unsubstituted, saturated or unsaturated C₁-C₆ alkyl. Each G¹, G¹′ and G¹″ may independently be a branched, unbranched, or aromatic cyclic or non-aromatic cyclic, substituted or unsubstituted, saturated or unsaturated C₁-C₅ alkyl. Each G¹, G¹′ and G¹″ may independently be a branched, unbranched, or non-aromatic cyclic, substituted or unsubstituted, saturated or unsaturated C₁-C₄ alkyl. Each G¹, G¹′ and G¹″ may independently be a branched, unbranched, or non-aromatic cyclic, substituted or unsubstituted, saturated or unsaturated C₁-C₃ alkyl. Each G¹, G¹′ and G¹″ may independently be a branched, unbranched, or non-aromatic cyclic, substituted or unsubstituted, saturated or unsaturated C₁-C₂ alkyl.

An optional substituent may be selected from the group consisting of oxo, OJ′″, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, OR⁵, CO₂R⁴, CONH₂, CONHR⁴, CONHR⁵, CONR⁴₂, NHR⁵, OPO₃H₃, CONR⁴R⁵, NR⁴R⁵, and NO₂. An optional substituent may be selected from the group consisting of: oxo (i.e. ═O), Or, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, and NO₂. An optional substituent may be selected from the group consisting of: oxo (i.e. ═O), Or, COOH, R⁴, OH, OR⁴, F, Cl, Br, I, NH₂, NHR⁴, NR⁴₂, SO₃H, SO₃R⁴, SO₂R⁴, and NO₂. An optional substituent may be selected from the group consisting of: oxo (i.e. ═O), Or, COOH, R⁴, OH, OR⁴, F, Cl, Br, I, NH₂, and NO₂. An optional substituent may be selected from the group consisting of: oxo (i.e. ═O), Or, COOH, R⁴, OH, OR⁴, F, Cl, Br, and I. An optional substituent may be selected from the group consisting of: oxo (i.e. ═O), Or, COOH, OH, F, Cl, Br, and I. An optional substituent may be selected from the group consisting of: oxo (i.e. ═O), Or, COOH, OH, F, and Cl. Each linear or branched, or aromatic cyclic or non-aromatic cyclic, saturated or unsaturated C₁-C₁₀ alkyl may be substituted with, for example, 1, 2, 3, 4, 5, or 6 substituents.

Each R⁴ may independently be unsubstituted C₁-C₁₀ alkyl. Each R⁴ may independently be unsubstituted C₁-C₉ alkyl. Each R⁴ may independently be unsubstituted C₁-C₈ alkyl. Each R⁴ may independently be unsubstituted C₁-C₇ alkyl. Each R⁴ may independently be unsubstituted C₁-C₆ alkyl. Each R⁴ may independently be unsubstituted C₁-C₅ alkyl. Each R⁴ may independently be unsubstituted C₁-C₄ alkyl. Each R⁴ may independently be unsubstituted C₁-C₃ alkyl. Each R⁴ may independently be unsubstituted C₁-C₂ alkyl. Each R⁴ may independently be unsubstituted C₁ alkyl. Each R⁴ may independently be unsubstituted C₂ alkyl. Each R⁴ may independently be unsubstituted C₃ alkyl. Each R⁴ may independently be unsubstituted C₄ alkyl. Each R⁴ may independently be unsubstituted Cₛ alkyl. Each R⁴ may independently be unsubstituted C₆ alkyl. Each R⁴ may independently be unsubstituted C₇ alkyl. Each R⁴ may independently be unsubstituted C₈ alkyl. Each R⁴ may independently be unsubstituted C₉ alkyl. Each R⁴ may independently be unsubstituted C₁₀ alkyl.

Each R⁵ may independently be C₁-C₁₀ acyl. Each R⁵ may independently be C₁-C₉ acyl. Each R⁵ may independently be C₁-C₈ acyl. Each R⁵ may independently be C₁-C₇ acyl. Each R⁵ may independently be C₁-C₆ acyl. Each R⁵ may independently be C₁-C₅ acyl. Each R⁵ may independently be C₁-C₄ acyl. Each R⁵ may independently be C₁-C₃ acyl. Each R⁵ may independently be C₁-C₂ acyl. Each R⁵ may independently be C₁ acyl. Each R⁵ may independently be C₂ acyl. Each R⁵ may independently be C₃ acyl. Each R⁵ may independently be C₄ acyl. Each R⁵ may independently be C₅ acyl. Each R⁵ may independently be C₆ acyl. Each R⁵ may independently be C₇ acyl. Each R⁵ may independently be C₈ acyl. Each R⁵ may independently be C₉ acyl. Each R⁵ may independently be C₁₀ acyl.

Each R may independently be C₁-C₁₀ acyl. Each R may independently be C₁-C₉ acyl. Each R may independently be C₁-C₅ acyl. Each R may independently be C₁-C₇ acyl. Each R may independently be C₁-C₆ acyl. Each R may independently be C₁-C₅ acyl. Each R may independently be C₁-C₄ acyl. Each R may independently be C₁-C₃ acyl. Each R may independently be C₁-C₂ acyl. Each R may independently be C₁ acyl. Each R may independently be C₂ acyl. Each R may independently be C₃ acyl. Each R may independently be C₄ acyl. Each R may independently be C₅ acyl. Each R may independently be C₆ acyl. Each R may independently be C₇ acyl. Each R may independently be C₅ acyl. Each R may independently be C₉ acyl. Each R may independently be C₁₀ acyl.

X may be a single bond, C═O, C(OH)₂, C(OR¹)₂, C(OH)(OR¹), C(OR¹)(OR²), O, S, SO, SO₂, C═NOH, C═N)R¹, CHNH₂, CHNHR¹, CHNHR², CHNR¹₂, CHNR²₂, or CHNR¹R². X may be C═O, C(OH)₂, C(OR¹)₂, C(OH)(OR¹), C(OR¹)(OR²), C═NOH, C═N)R¹, CHNH₂, CHNHR¹, CHNHR², CHNR¹₂, CHNR²₂, or CHNR¹R². X may be C═O, C(OH)₂, C(OR¹)₂, C(OH)(OR¹), or C(OR¹)(OR²). X may be C═NOH, C═N)R¹, CHNH₂, CHNHR¹, CHNHR², CHNR¹₂, CHNR²₂, or CHNR¹R². X may be S, SO, or SO₂. X may be a single bond, C═O, O, S, or SO. X may be a single bond, C═O, O, or S. X may be a single bond, O, or S. X may be a single bond. X may be C═O. X may be C(OH)₂. X may be C(OR¹)₂. X may be C(OH)(OR¹). X may be C(OR¹)(OR²). X may be O. X may be S. X may be SO. X may be SO₂. X may be C═NOH. X may be C═N)R¹. X may be CHNH₂. X may be CHNHR¹. X may be CHNHR². X may be CHNR¹₂. X may be CHNR²₂. X may be CHNR¹R².

Each R¹ may independently be linear or branched, substituted or unsubstituted, saturated or unsaturated C₁-C₁₀ alkyl, or two of the R¹ groups may be joined to form a cyclic ketal. Each R¹ may independently be linear or branched, substituted or unsubstituted, saturated or unsaturated C₁-C₁₀ alkyl. Each R¹ may independently be branched or unbranched, substituted or unsubstituted, saturated or unsaturated C₁-C₉ alkyl. Each R¹ may independently be branched or unbranched, substituted or unsubstituted, saturated or unsaturated C₁-C₈ alkyl. Each R¹ may independently be branched or unbranched, substituted or unsubstituted, saturated or unsaturated C₁-C₇ alkyl. Each R¹ may independently be branched or unbranched, substituted or unsubstituted, saturated or unsaturated C₁-C₆ alkyl. Each R¹ may independently be branched or unbranched, substituted or unsubstituted, saturated or unsaturated C₁-C₅ alkyl. Each R¹ may independently be branched or unbranched, substituted or unsubstituted, saturated or unsaturated C₁-C₄ alkyl. Each R¹ may independently be branched or unbranched, substituted or unsubstituted, saturated or unsaturated C₁-C₃ alkyl. Each R¹ may independently be branched or unbranched, substituted or unsubstituted, saturated or unsaturated C₁-C₂ alkyl. Each R¹ may independently be branched or unbranched, substituted or unsubstituted, saturated or unsaturated C₁ alkyl. Each R¹ may independently be branched or unbranched, substituted or unsubstituted, saturated or unsaturated C₂ alkyl. Each R¹ may independently be branched or unbranched, substituted or unsubstituted, saturated or unsaturated C₃ alkyl. Each R¹ may independently be branched or unbranched, substituted or unsubstituted, saturated or unsaturated C₄ alkyl. Each R¹ may independently be branched or unbranched, substituted or unsubstituted, saturated or unsaturated C₅ alkyl. Each R¹ may independently be branched or unbranched, substituted or unsubstituted, saturated or unsaturated C₆ alkyl. Each R¹ may independently be branched or unbranched, substituted or unsubstituted, saturated or unsaturated C₇ alkyl. Each R¹ may independently be branched or unbranched, substituted or unsubstituted, saturated or unsaturated C₈ alkyl. Each R¹ may independently be branched or unbranched, substituted or unsubstituted, saturated or unsaturated C₉ alkyl. Each R¹ may independently be branched or unbranched, substituted or unsubstituted, saturated or unsaturated C₁₀ alkyl. Each R¹ may be CH₃. Two of the R¹ groups may be joined to form a cyclic ketal.

Each R² may independently be C₁-C₁₀ acyl. Each R² may independently be C₁-C₉ acyl. Each R² may independently be C₁-C₈ acyl. Each R² may independently be C₁-C₇ acyl. Each R² may independently be C₁-C₆ acyl. Each R² may independently be C₁-C₅ acyl. Each R² may independently be C₁-C₄ acyl. Each R² may independently be C₁-C₃ acyl. Each R² may independently be C₁-C₂ acyl. Each R² may independently be C₁ acyl. Each R² may independently be C₂ acyl. Each R² may independently be C₃ acyl. Each R² may independently be C₄ acyl. Each R² may independently be C₅ acyl. Each R² may independently be C₆ acyl. Each R² may independently be C₇ acyl. Each R² may independently be C₈ acyl. Each R² may independently be C₉ acyl. Each R² may independently be C₁₀ acyl.

An optional substituent may be selected from the group consisting of oxo, Or, COOH, R³, OH, OR³, F, Cl, Br, I, NH₂, NHR³, NR³₂, CN, SH, SR³, SO₃H, SO₃R³, SO₂R³, OSO₃R³, OR⁶, CO₂R³, CONH₂, CONHR³, CONHR⁶, CONR³₂, NHR⁶, OPO₃H₃, CONR³R⁶, NR³R⁶, and NO₂. An optional substituent may be selected from the group consisting of: oxo (i.e. ═O), Or, COOH, R³, OH, OR³, F, Cl, Br, I, NH₂, NHR³, NR³₂, CN, SH, SR³, SO₃H, SO₃R³, SO₂R³, OSO₃R³, and NO₂. An optional substituent may be selected from the group consisting of: oxo (i.e. ═O), Or, COOH, R³, OH, OR³, F, Cl, Br, I, NH₂, NHR³, NR³₂, SO₃H, SO₃R³, SO₂R³, and NO₂. An optional substituent may be selected from the group consisting of: oxo (i.e. ═O), Or, COOH, R³, OH, OR³, F, Cl, Br, I, NH₂, and NO₂. An optional substituent may be selected from the group consisting of: oxo (i.e. ═O), Or, COOH, R³, OH, OR³, F, Cl, Br, and I. An optional substituent may be selected from the group consisting of: oxo (i.e. ═O), Or, COOH, OH, F, Cl, Br, and I. An optional substituent may be selected from the group consisting of: oxo (i.e. ═O), OJ′″, COOH, OH, F, and Cl. Each linear or branched, or aromatic cyclic or non-aromatic cyclic, saturated or unsaturated C1-C10 alkyl may be substituted with, for example, 1, 2, 3, 4, 5, or 6 substituents.

Each R³ may independently be unsubstituted C₁-C₁₀ alkyl. Each R³ may independently be unsubstituted C₁-C₉ alkyl. Each R³ may independently be unsubstituted C₁-C₈ alkyl. Each R³ may independently be unsubstituted C₁-C₇ alkyl. Each R³ may independently be unsubstituted C₁-C₆ alkyl. Each R³ may independently be unsubstituted C₁-C₅ alkyl. Each R³ may independently be unsubstituted C₁-C₄ alkyl. Each R³ may independently be unsubstituted C₁-C₃ alkyl. Each R³ may independently be unsubstituted C₁-C₂ alkyl. Each R³ may independently be unsubstituted C₁ alkyl. Each R³ may independently be unsubstituted C₂ alkyl. Each R³ may independently be unsubstituted C₃ alkyl. Each R³ may independently be unsubstituted C₄ alkyl. Each R³ may independently be unsubstituted C₅ alkyl. Each R³ may independently be unsubstituted C₆ alkyl. Each R³ may independently be unsubstituted C₇ alkyl. Each R³ may independently be unsubstituted C₈ alkyl. Each R³ may independently be unsubstituted C₉ alkyl. Each R³ may independently be unsubstituted C₁₀ alkyl.

Each R⁶ may independently be C₁-C₁₀ acyl. Each R⁶ may independently be C₁-C₉ acyl. Each R⁶ may independently be C₁-C₈ acyl. Each R⁶ may independently be C₁-C₇ acyl. Each R⁶ may independently be C₁-C₆ acyl. Each R⁶ may independently be C₁-C₅ acyl. Each R⁶ may independently be C₁-C₄ acyl. Each R⁶ may independently be C₁-C₃ acyl. Each R⁶ may independently be C₁-C₂ acyl. Each R⁶ may independently be C₁ acyl. Each R⁶ may independently be C₂ acyl. Each R⁶ may independently be C₃ acyl. Each R⁶ may independently be C₄ acyl. Each R⁶ may independently be C₅ acyl. Each R⁶ may independently be C₆ acyl. Each R⁶ may independently be C₇ acyl. Each R⁶ may independently be C₈ acyl. Each R⁶ may independently be C₉ acyl. Each R⁶ may independently be C₁₀ acyl.

X may be a single bond. X may be C═O. X may be C(OH)₂, C(OR¹)₂, C(OR²)₂, C(OH)(OR¹), C(OH)(OR²), or C(OR¹)(OR²). X may be O, S, SO, or SO₂. X may be O. X may be S. X may be C═NOH. X may be CHNH₂, CHNHR¹, CHNHR², CHNR¹₂, CHNR²₂, or CHNR¹R². Each remaining Z may be independently CH, CF, CCl, CBr, or Cl. Each remaining Z may be CH. J may be O. J² may be O. Each M² may independently be H, CH₃, CH₂Cl, CH₂Br, CH₂OJ″, CH₂OG¹, CH₂OG¹OG¹′, G¹OG¹′, G¹OG¹′OG¹″, CH₂SG¹, CH₂NH₂, CH₂NHG¹, or CH₂NG¹₂; and each remaining Z may independently be N, CH, CF, CCl, CBr, CI or COH. M² may be H, CH₃, CH₂Cl, CH₂Br, CH₂OJ″, CH₂OG¹, or CH₂OG¹OG¹′. M² may be CH₂Cl, CH₂Br, CH₂OH, CH₂OCH₃, CH₂O(isopropyl), or CH₂OC₂H₄OC₄H₉. M² may be H, CH₃, CH₂Cl, CH₂OJ″, CH₂OG¹, or CH₂OG¹OG¹′. M² may be CH₂Cl, CH₂OH, CH₂OCH₃, CH₂O(isopropyl), or CH₂OC₂H₄OC₄H₉. J may be O; n may be is 0, 1, 2, 3, 4, 5, 6, 7, or 8; M may be CH₂Cl; L may be A-D; A may be O; and D may be H. J² may be O; m may be 0, 1, 2, 3, 4, 5, 6, 7, or 8; M² may be CH₂Cl; L² may be A²-D²; A² may be O; and D² may be H. J may be O; n may be 1; M may be CH₂Cl; L may be A-D; A may be O; and D may be H. J² may be O; m may be 1; M² may be CH₂Cl; L² may be A2-D²; A² may be O; and D² may be H. J may be 0; n may be 0, 1, 2, 3, 4, 5, 6, 7, or 8; M may be CH₂Cl; L may be H. J² may be O; m may be 0, 1, 2, 3, 4, 5, 6, 7, or 8; M² may be CH₂Cl; L² may be H. J may be O; n may be 1; M may be CH₂Cl; L may be H. J² may be O; m may be 1; M² may be CH₂Cl; L² may be H. J may be O; n may be 0, 1, 2, 3, 4, 5, 6, 7, or 8; M may be H; L may be A-D; A may be O; and D may be H. J² may be O; m may be 0, 1, 2, 3, 4, 5, 6, 7, or 8; M² may be H; L² may be A²-D²; A² may be O; and D² may be H. J may be O; n may be 1; M may be H; L may be A-D; A may be O; and D may be H. J² may be O; m may be 1; M² may be H; L² may be A²-D²; A² may be O; and D² may be H. J may be O; n may be 0, 1, 2, 3, 4, 5, 6, 7, or 8; M may be H; L may be A-D; A may be O; and D may be

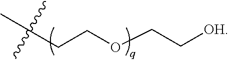

### may be O; n may be 0, 1, 2, 3, 4, 5, 6, 7, or 8; M may be H; L may be A-D; A may be O; and D may be

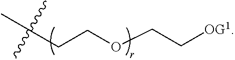

### J may be O; n may be 0, 1, 2, 3, 4, 5, 6, 7, or 8; M may be H; L may be A-D; A may be O; and D may be

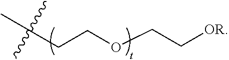

### J² may be O; m may be 0, 1, 2, 3, 4, 5, 6, 7, or 8; M² may be H; L² may be A²-D²; A² may be O; and D² may be

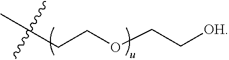

### J² may be O; m may be 0, 1, 2, 3, 4, 5, 6, 7, or 8; M² may be H; L² may be A²-D²; A² may be O; and D² may be

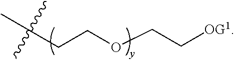

### J² may be O; m may be 0, 1, 2, 3, 4, 5, 6, 7, or 8; M² may be H; L² may be A²-D²; A² may be O; and D² may be

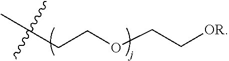

### J may be O; n may be 1; M may be H; L may be A-D; A may be O; and D may be

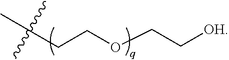

### J may be O; n may be 1; M may be H; L may be A-D; A may be O; and D may be

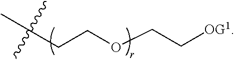

### J may be O; n may be 1; M may be H; L may be A-D; A may be O; and D may be

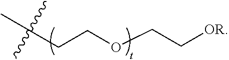

### J² may be O; m may be 1; M² may be H; L² may be A²-D²; A² may be O; and D² may be

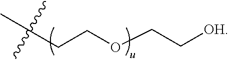

### J² may be O; m may be 1; M² may be H; L² may be A²-D²; A² may be O; and D² may be

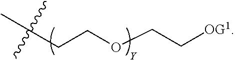

### J² may be O; m may be 1; M² may be H; L² may be A²-D²; A² may be O; and D² may be

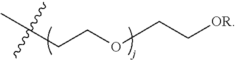

J may be O; n may be 0; M may be C═CH; and L may be H. J² may be O; m may be 0; M² may be C≡CH; and L² may be H. J may be O; n may be 1; M may be C≡CH; L may be A-D; A may be O; and D may be H. J² may be O; m may be 1; M² may be C≡CH; L² may be A²-D²; A² may be O; and D² may be H.

In accordance with another embodiment, there is provided a use of the compounds having a structure of any one of the Formula I to XXI for preparation of a medicament for modulating androgen receptor (AR).

In accordance with another embodiment, there are provided the compounds having a structure of any one of the Formula I to XXI for modulating androgen receptor (AR).

In accordance with another embodiment, there is provided a pharmaceutical composition comprising a compound having a structure of any one of the Formula I to XXI set out above and a pharmaceutically acceptable excipient.

In accordance with another embodiment, there is provided a method for modulating AR activity, the method comprising administering to a mammalian cell a compound having a structure of any one of the Formula I to XXI set out above.

The modulating of the androgen receptor (AR) activity may be in a mammalian cell. The modulating of the androgen receptor (AR) activity may be in a mammal. The mammal may be a human.

Alternatively, the administering may be to a mammal. The administering may be to a mammal in need thereof and in an effective amount for the treatment of at least one indication selected from the group consisting of: prostate cancer, breast cancer, ovarian cancer, endometrial cancer, hair loss, acne, hirsutism, ovarian cysts, polycystic ovary disease, precocious puberty, and age-related macular degeneration.

The compounds described herein are meant to include all racemic mixtures and all individual enantiomers or combinations thereof, whether or not they are represented herein. Alternatively, one or more of the OH groups on the above compounds may be substituted to replace the H with a moiety selected from TABLE 1.

The mammalian cell may be a human cell. The modulating AR activity may be for inhibiting AR N-terminal domain activity. The modulating AR activity may be for inhibiting AR activity. The modulating may be in vivo. The modulating AR activity may be for treatment of at least one indication selected from the group consisting of: prostate cancer, breast cancer, ovarian cancer, endometrial cancer, hair loss, acne, hirsutism, ovarian cysts, polycystic ovary disease, precocious puberty, and age-related macular degeneration. The indication may be prostate cancer. The prostate cancer may be androgen-independent prostate cancer. The prostate cancer may be androgen-dependent prostate cancer.

The at least one Z of the other aromatic ring may be selected from: C-T; CG¹; COG¹; CNHG¹; COSO₃H; COPO₃H₂; CSG¹; CSOG¹; CSO₂G′; and CNG¹₂. The at least one Z of the other aromatic ring may be selected from: C-T; COG¹; CNHG¹; and CG¹. The remaining Z may be independently selected from: N; CG¹; CH; CF; CCl; CBr; and CI. Each of the remaining Z may be independently selected from: CG¹; CH; CCl; and CBr. Each of the remaining Z may be independently selected from: CG¹; CH; and CBr. CG¹ may be CCH₃. Each of the remaining Z may be independently selected from: CCH₃; CH; and CBr. J may be selected from: O; S; SO; NH; NG¹; and SO₂. J may be selected from: O; NH; NG¹; and S. J may be O. M may be selected from: H; Cl; Br; CH₂Cl; CHCl₂; CH₂Br; CHBr₂; CH₂OG¹; and C≡CH. M may be selected from: Cl; Br; CH₂Cl; CH₂Br; CH₂OG¹; and C≡CH. M may be selected from: CH₂OG¹; Cl; Br; CH₂Cl; and CH₂Br. M may be selected from: CH₂Cl; CH₂Br; and C≡CH. M may be CH₂Cl. L may be H. L may be A-D. A may be selected from: O; S; and NH. A may be O. D may be selected from: H;

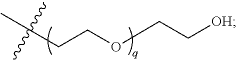

and a moiety selected from TABLE 1. J² may be selected from: O; S; SO; NH; and SO₂. J² may be selected from: 0; and S. J² may be O. M² may be selected from: H; Cl; Br; CH₂Cl; CHCl₂; CH₂Br; CHBr₂; CH₂OH; CH₂OJ″; CH₂OG¹; and C≡CH. M² may be selected from: H; Cl; Br; CH₂Cl; CH₂Br; CH₂OG¹; and C≡CH. M² may be selected from: Cl; Br; CH₂OG¹; CH₂Cl; and CH₂Br. M² may be selected from: CH₂OG¹; CH₂Cl; CH₂Br; and C≡CH. M² may be CH₂Cl. L² may be H. L² may be A²-D². A² may be selected from: O; S; and NH. A² may be O. D² may be selected from: H;

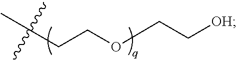

and a moiety selected from TABLE 1. Each G¹ G¹′ and G¹′″ may be independently a linear or branched, substituted or unsubstituted, saturated or unsaturated C₁-C₁₀ alkyl, wherein the optional substituent may be selected from the group consisting of: oxo; Or; COOH; OH; F; Cl; Br; I; NH₂; CN; SH; SO₃H; CONH₂; OPO₃H₃; and NO₂ Each G′ G¹′ and G¹′″ may be independently a linear or branched, substituted or unsubstituted, saturated or unsaturated C₁-C₁₀ alkyl, wherein the optional substituent may be selected from the group consisting of oxo; Or; COOH; OH; Cl; Br; NH₂; and NO₂. X may be selected from the group consisting of: a single bond; C═O; O; S; SO; and SO₂. X may be selected from the group consisting of: a single bond; C═O; O; and S.

In accordance with another embodiment, there is provided a use of a compound selected from TABLE 3 (for example, EPI-100, EPI-101, EPI-102, EPI-106, EPI-107, EPI-108, EPI-109, EPI-111, EPI-114, EPI-116, EPI-117, EPI-300, EPI-400 and EPI-3000) for modulating androgen receptor (AR) activity.

In accordance with another embodiment, there is provided a use of a compound selected from TABLE 2 for modulating androgen receptor (AR) activity.

In accordance with another embodiment, there is provided a use of a compound having a structure of Formula V

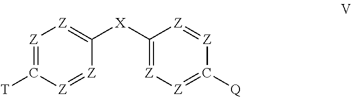

or a pharmaceutically acceptable salt thereof, wherein: Q may be selected from

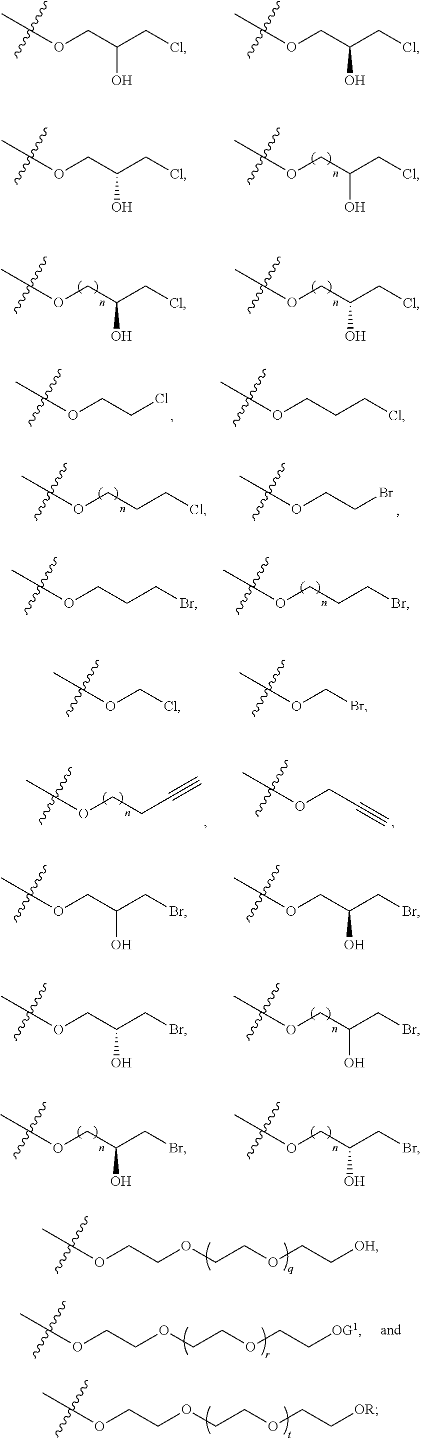

may be 0, 1, 2, 3, 4, 5, 6, 7 or 8; each of q, r and t may be independently 0, 1, 2, 3, 4, 5, 6or 7; T may be selected from

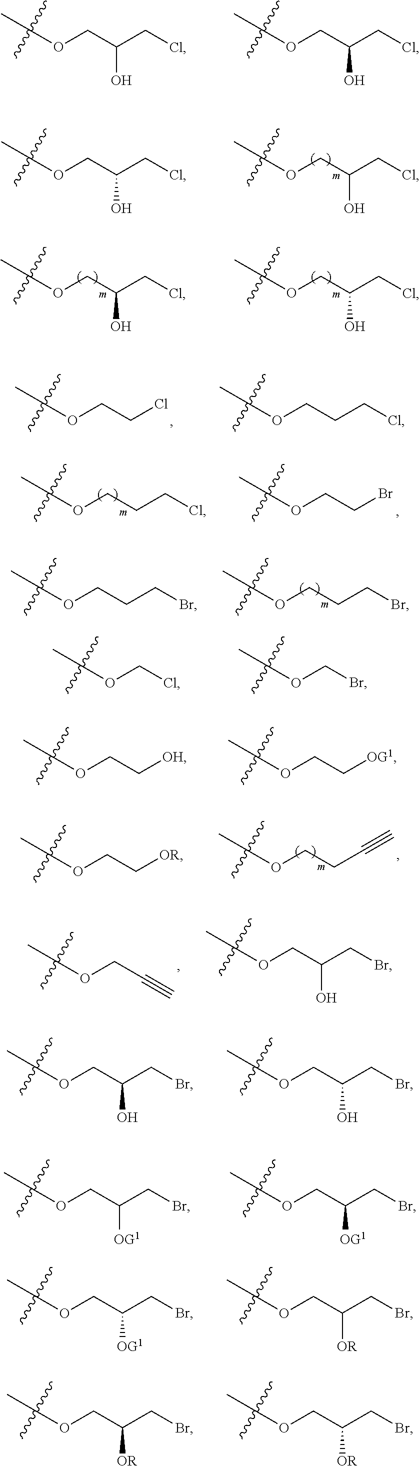

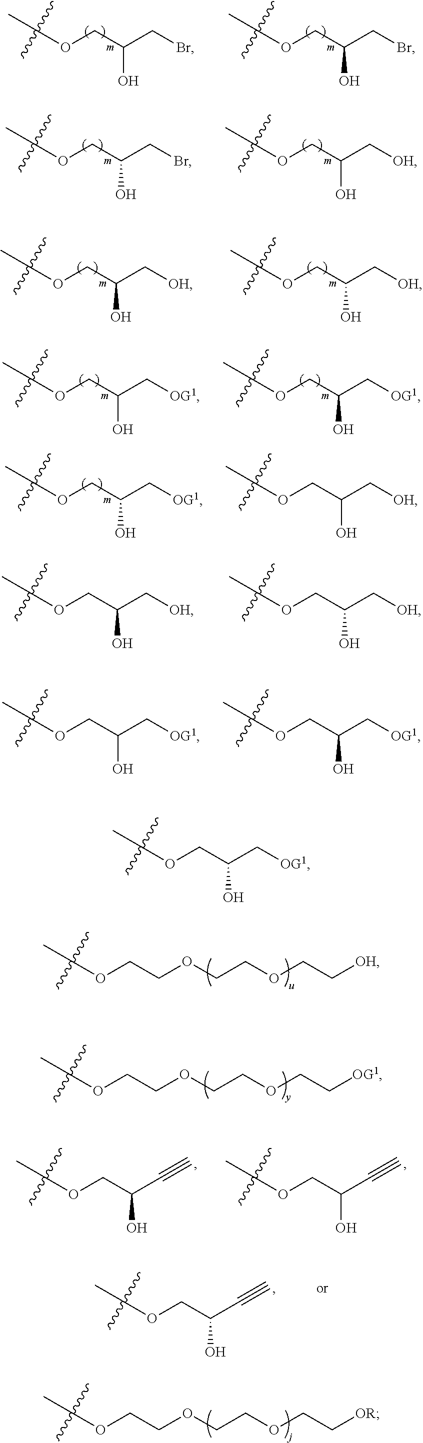

m may be 0, 1, 2, 3, 4, 5, 6, 7 or 8; each of u, y and j may be independently 0, 1, 2, 3, 4, 5, 6 or 7; X may be selected from the group consisting of: C═O, O, S, and SO; each Z may be independently selected from: N; CG¹; CH; CF; CCl; CBr; and CI; and each G¹ may be a linear or branched, substituted or unsubstituted, saturated or unsaturated C₁-C₁₀ alkyl, wherein the optional substituent may be selected from the group consisting of: oxo; Or; COOH; OH; F; Cl; Br; I; NH₂; CN; SH; SO₃H; CONH₂; OPO₃H₃; and NO₂, for modulating androgen receptor (AR) activity.

In accordance with another embodiment, there is provided a method of modulating androgen receptor (AR) activity, the method including administration of a compound or pharmaceutically acceptable salt thereof described herein or a compound having a structure of Formula I or V as set out herein to a subject in need thereof.

In accordance with another embodiment, there is provided a pharmaceutical composition comprising a compound or pharmaceutically acceptable salt described herein or a compound having a structure of Formula I or V as set out herein; and a pharmaceutically acceptable carrier.

The modulation of androgen receptor (AR) activity may be for the treatment of one or more of the following: prostate cancer; breast cancer; ovarian cancer; endometrial cancer; hair loss; acne; hirsutism; ovarian cysts; polycystic ovary disease; precocious puberty; and age-related macular degeneration.

In accordance with another embodiment, there are provided compounds having a structure of Formula I

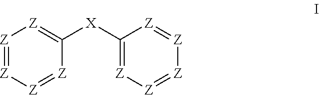

or a pharmaceutically acceptable salt thereof, wherein: X may be C═O, C(OH)₂, C(OR¹)₂, C(OH)(OR¹), C(OR¹)(OR²), O, S, SO, C═NOH, C═N)R¹, CHNH₂, CHNHR¹, CHNHR², CHNR¹₂, CHNR²₂, or CHNR¹R²; each R¹ may be independently linear or branched, substituted or unsubstituted, saturated or unsaturated C₁-C₁₀ alkyl, and each R² may be independently C₁-C₁₀ acyl, or two of the R¹ groups are joined to form a cyclic ketal, wherein the optional substituent may be selected from the group consisting of oxo, Or, COOH, R³, OH, OR³, F, Cl, Br, I, NH₂, NHR³, NR³₂, CN, SH, SR³, SO₃H, SO₃R³, SO₂R³, OSO₃R³, OR⁶, CO₂R³, CONH₂, CONHR³, CONHR⁶, CONR³₂, NHR⁶, OPO₃H₃, CONR³R⁶, NR³R⁶, and NO₂; each R³ may be independently unsubstituted C₁-C₁₀ alkyl; each R⁶ may be independently C₁-C₁₀ acyl; at least one Z of one aromatic ring may be independently C-Q, at least one Z of the other aromatic ring may be independently C-T, CF, CCl, CI, COH, CG¹, CNH₂, CNG¹₂, COSO₃H, COPO₃H₂, CSG¹, CSOG¹, or CSO₂G¹, and each remaining Z may be independently C-T, N, CH, CF, CCl, CBr, CI, COH, CG¹, COG¹, CNH₂, CNHG¹, CNG¹₂, COSO₃H, COPO₃H₂, CSG¹, CSOG¹, or CSO₂G¹; Q may be

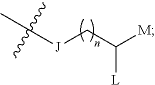

J may be G¹, O, CH₂, CHG¹, CG¹₂, SO, or NR; M may be H, Cl, Br, CH₂Cl, CHCl₂, CCl₃, CH₂Br, CHBr₂, CBr₃, or C≡CH; L may be H or A-D; A may be O, S, NH, NG¹, N⁺H₂, or N⁺HG¹; D may be H, G¹, R,

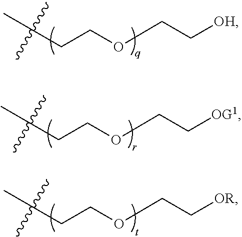

, or a moiety selected from TABLE 1; each of q, r and t may be independently 0, 1, 2, 3, 4, 5, 6 or 7;

n may be 0, 1, 2, 3, 4, 5, 6, 7 or 8; T may be

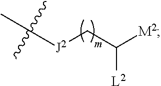

J² may be G¹, O, CH₂, CHG¹, CG¹₂, S, NH, SO, SO₂, or NR; M² may be H, CH₃, Cl, Br, CH₂Cl, CHCl₂, CCl₃, CH₂Br, CHBr₂, CBr₃, CH₂OH, CH₂OJ″, G¹, CH₂OG¹, CH₂OR, CH₂OG¹OG¹′, G¹OG¹′, G¹OG¹′OG¹″, CH₂SG¹, CH₂NH₂, CH₂N⁺HG¹, CH₂NG¹₂, or C≡CH; L² may be H or A²-D²; A² may be O, S, SO, SO₂, NH, NG¹, N⁺H₂, or N⁺HG¹; D² may be H, G¹, R,

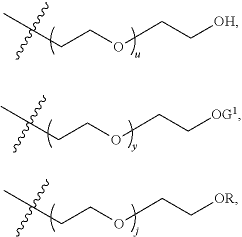

or a moiety selected from TABLE 1; each of u, y and j may be independently 0, 1, 2, 3, 4, 5, 6 or 7; m may be 0, 1, 2, 3, 4, 5, 6, 7 or 8; each of J″ and J′″ may be independently a moiety selected from TABLE 1; each G′ G¹′ and G¹′″ may be independently linear or branched, substituted or unsubstituted, saturated or unsaturated C₁-C₁₀ alkyl, wherein the optional substituent may be selected from the group consisting of oxo, Or, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, OR⁵, CO₂R⁴, CONH₂, CONHR⁴, CONHR⁵, CONR⁴₂, NHR⁵, OPO₃H₃, CONR⁴R⁵, NR⁴R⁵, and NO₂; each R⁴ may be independently unsubstituted C₁-C₁₀ alkyl; each R⁵ may be independently C₁-C₁₀ acyl; and R may be C₁-C₁₀ acyl.

The Z of the other aromatic ring may be selected from: C-T; CF; CCl; CBr; CI; CG¹; CNH₂; and CNG¹₂. The at least one Z of the other aromatic ring may be selected from: C-T; and CG¹. Each of the remaining Z may be independently selected from: N; CG¹; CH; CF; CCl; and CI. Each of the remaining Z may be independently selected from: CG¹; CH; and CCl. Each of the remaining Z may be independently selected from: CG¹; and CH. CG¹ may be CCH₃. Each remaining Z may be independently selected from: CCH₃; and CH. J may be selected from: 0; and SO. J may be O. M may be selected from: H; Cl; Br; CH₂Cl; CHCl₂; CH₂Br; CHBr₂; CH₂OG¹; and C≡CH. M may be selected from: H; Cl; Br; CH₂Cl; CH₂Br; CH₂OG¹; and C═CH. M may be selected from: Cl; Br; CH₂Cl; and CH₂Br. M may be selected from: CH₂Cl; CH₂Br; and M may be CH₂Cl. L may be H. L may be A-D. A may be selected from: O; S; and NH. A may be O. D may be selected from: H;

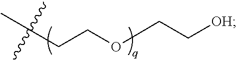

and a moiety selected from TABLE 1. J² may be selected from: O; S; SO; NH; and SO₂. J² may be selected from: 0; and S. J² may be O. M² may be selected from: H; Cl; Br; CH₂Cl; CHCl₂; CH₂Br; CHBr₂; CH₂OH; CH₂OJ″; CH₂OG¹; and M² may be selected from: H; Cl; Br; CH₂Cl; CH₂Br; CH₂OG¹; and C≡CH. M² may be selected from: Cl; Br; CH₂OG¹; CH₂Cl; and CH₂Br. M² may be selected from: CH₂Cl; CH₂Br; and C≡CH. M² may be CH₂Cl. L² may be H. L² may be A²-D². A² may be selected from: O; S; and NH. A² may be O. D² may be selected from: H;

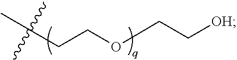

and a moiety selected from TABLE 1. Each G¹ G¹′ and G¹′″ may be independently a linear or branched, substituted or unsubstituted, saturated or unsaturated C₁-C₁₀ alkyl, wherein the optional substituent may be selected from the group consisting of: oxo; OJ′″; COOH; OH; F; Cl; Br; I; NH₂; CN; SH; SO₃H; CONH₂; OPO₃H₃; and NO₂. Each G¹′″ and may be independently a linear or branched, substituted or unsubstituted, saturated or unsaturated C₁-C₁₀ alkyl, wherein the optional substituent may be selected from the group consisting of: oxo; Or; COOH; OH; Cl; Br; NH₂; and NO₂ X may be selected from the group consisting of: C═O; O; S; and SO.

In accordance with another embodiment, there are provided compounds selected from TABLE 3 (for example, EPI-100, EPI-101, EPI-102, EPI-106, EPI-107, EPI-108, EPI-109, EPI-111, EPI-114, EPI-116, EPI-117, EPI-300, and EPI-400, but excluding EPI-3000).

In accordance with another embodiment, there are provided compounds selected from TABLE 2.

In accordance with another embodiment, there are provided compounds having a structure of Formula V

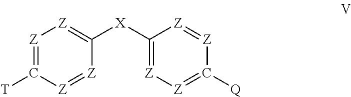

or a pharmaceutically acceptable salt thereof, wherein: Q may be selected from

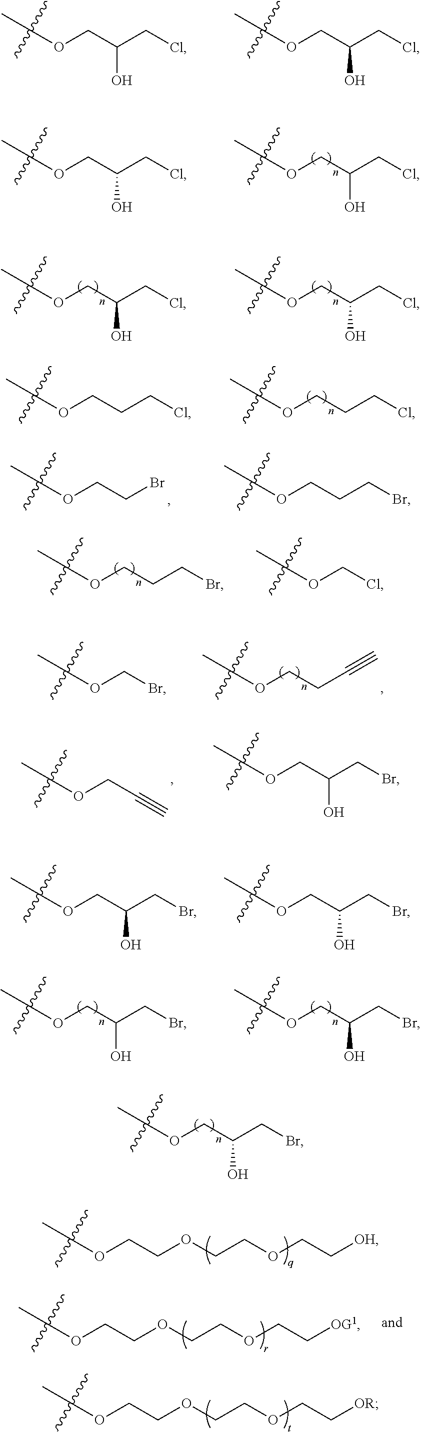

may be 1, 2, 3, 4, 5, 6, 7 or 8; n may be 0, 1, 2, 3, 4, 5, 6, 7 or 8; each of q, r and t may be independently 0, 1, 2, 3, 4, 5, 6 or 7; T may be selected from

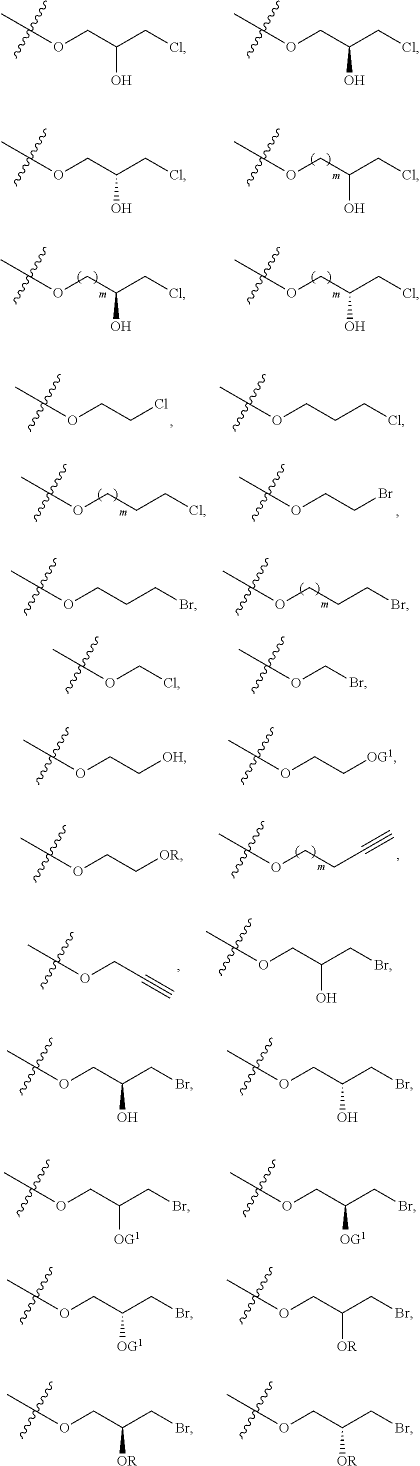

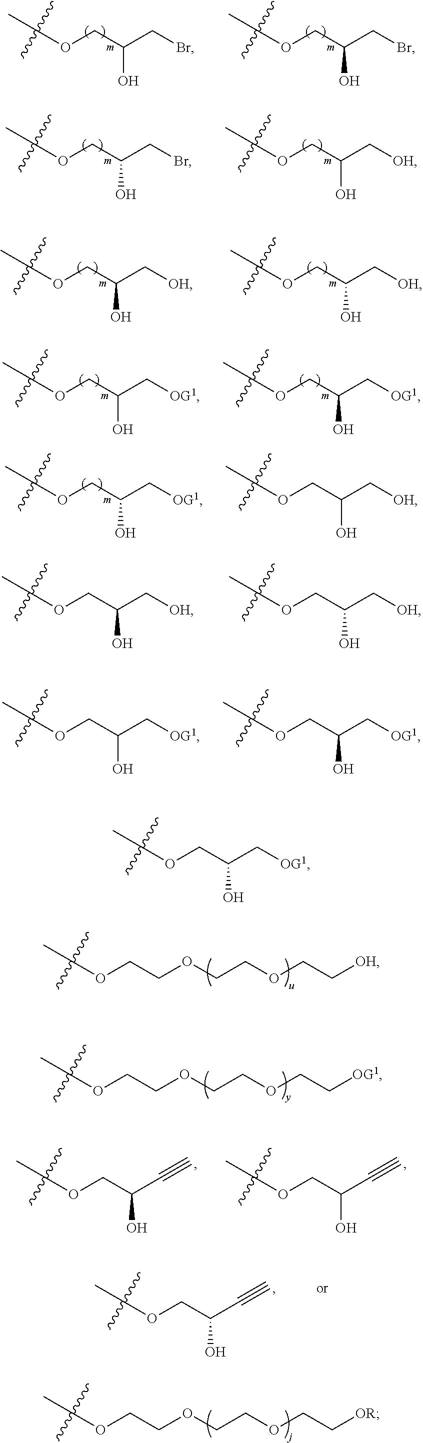

m may be 0, 1, 2, 3, 4, 5, 6, 7 or 8; each of u, y and j may be independently 0, 1, 2, 3, 4, 5, 6 or 7; X may be selected from the group consisting of: C═O, O, S, and SO; each Z may be independently selected from: N; CG¹; CH; CF; CCl; CBr; and CI; and each G¹ may be a linear or branched, substituted or unsubstituted, saturated or unsaturated C₁-C₁₀ alkyl, wherein the optional substituent may be selected from the group consisting of: oxo; Or; COOH; OH; F; Cl; Br; I; NH₂; CN; SH; SO₃H; CONH₂; OPO₃H₃; and NO₂.

In accordance with another embodiment, there are provided pharmaceutical compositions for treating one or more of the following: prostate cancer; breast cancer; ovarian cancer; endometrial cancer; hair loss; acne; hirsutism; ovarian cysts; polycystic ovary disease; precocious puberty; and age-related macular degeneration, the pharmaceutical composition comprising a compound described herein and a pharmaceutically acceptable carrier.

The compounds described herein are meant to include all racemic mixtures and all individual enantiomers or combinations thereof, whether or not they are represented herein. Alternatively, one or more of the OH groups may be substituted to replace the H with a moiety selected from TABLE 1.

In accordance with another embodiment, there is provided a pharmaceutical composition comprising a compound according to any one of the above compounds and a pharmaceutically acceptable excipient.

In accordance with a further embodiment, there is provided a method of screening for androgen receptor modulating compounds, wherein the compounds screened are selected from the compounds as described anywhere herein.

### BRIEF DESCRIPTION OF THE DRAWINGS

FIG. 1 shows a dose response for EPI-100, EPI-101, and EPI-102 as compared to EPI-001 in a LNCaP PSA (6.1 kb)-luciferase assay.

FIG. 2A shows a dose response for EPI-109, EPI-101 and EPI-111 as compared to EPI-001 in a LNCaP PSA (6.1 kb)-luciferase assay.

FIG. 2B shows a dose response for EPI-109 and EPI-101 as compared to EPI-001 in a LNCaP PSA (6.1 kb)-luciferase assay.

FIG. 3A shows a dose response for EPI-107 as compared to EPI-001 in a LNCaP PSA (6.1 kb)-luciferase assay.

FIG. 3B shows a dose response for EPI-108 as compared to EPI-001 in a LNCaP PSA (6.1 kb)-luciferase assay.

FIG. 4A shows a dose response for EPI-114 as compared to EPI-001 in a LNCaP PSA (6.1 kb)-luciferase assay.

FIG. 4B shows a dose response for EPI-116 as compared to EPI-001 in a LNCaP PSA (6.1 kb)-luciferase assay.

FIG. 5A shows a dose response for EPI-106 and EPI-117 as compared to EPI-001 in a LNCaP PSA (6.1 kb)-luciferase assay.

FIG. 5B shows a dose response for EPI-400 and EPI-108 as compared to EPI-001 in a LNCaP PSA (6.1 kb)-luciferase assay.

FIG. 6A shows a dose response for EPI-300 as compared to EPI-001 in a LNCaP PSA (6.1 kb)-luciferase assay.

FIG. 6B shows a dose response for EPI-300 and EPI-3000 in a LNCaP PSA (6.1 kb)-luciferase assay.

### DETAILED DESCRIPTION

As used herein, the phrase “Cₓ—C_{y} alkyl” is used as it is normally understood to a person of skill in the art and often refers to a chemical entity that has a carbon skeleton or main carbon chain comprising a number from x to y (with all individual integers within the range included, including integers x and y) of carbon atoms. For example a “C₁-C₁₀ alkyl” is a chemical entity that has 1, 2, 3, 4, 5, 6, 7, 8, 9 or 10 carbon atom(s) in its carbon skeleton or main chain.

As used herein, the term “cyclic C_{X}-C_{y} alkyl” is used as it is normally understood to a person of skill in the art and often refers to a compound or a chemical entity in which at least a portion of the carbon skeleton or main chain of the chemical entity is bonded in such a way so as to form a ‘loop’, circle or ring of atoms that are bonded together. The atoms do not have to all be directly bonded to each other, but rather may be directly bonded to as few as two other atoms in the ‘loop’. Non-limiting examples of cyclic alkyls include benzene, toluene, cyclopentane, bisphenol and 1-chloro-3-ethylcyclohexane.

As used herein, the term “branched” is used as it is normally understood to a person of skill in the art and often refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl are tert-butyl and isopropyl.

As used herein, the term “unbranched” is used as it is normally understood to a person of skill in the art and often refers to a chemical entity that comprises a skeleton or main chain that does not split off into more that one contiguous chain. Non-limiting examples of unbranched alkyls are methyl, ethyl, n-propyl, and n-butyl.

As used herein, the term “substituted” is used as it is normally understood to a person of skill in the art and often refers to a chemical entity that has one chemical group replaced with a different chemical group that contains one or more heteroatoms. Unless otherwise specified, a substituted alkyl is an alkyl in which one or more hydrogen atom(s) is/are replaced with one or more atom(s) that is/are not hydrogen(s). For example, chloromethyl is a non-limiting example of a substituted alkyl, more particularly an example of a substituted methyl. Aminoethyl is another non-limiting example of a substituted alkyl, more particularly it is a substituted ethyl.

As used herein, the term “unsubstituted” is used as it is normally understood to a person of skill in the art and often refers to a chemical entity that is a hydrocarbon and/or does not contain a heteroatom. Non-limiting examples of unsubstituted alkyls include methyl, ethyl, tert-butyl, and pentyl.

As used herein, the term “saturated” when referring to a chemical entity is used as it is normally understood to a person of skill in the art and often refers to a chemical entity that comprises only single bonds. Non-limiting examples of saturated chemical entities include ethane, tert-butyl, and N⁺H₃.

As used herein, C₁-C₁₀ alkyl may include, for example, and without limitation, saturated C₁-C₁₀ alkyl, C₂-C₁₀ alkenyl and C₂-C₁₀ alkynyl. Non-limiting examples of saturated C₁-C₁₀ alkyl may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl and t-decyl. Non-limiting examples of C₂-C₁₀ alkenyl may include vinyl, allyl, isopropenyl, 1-propene-2-yl, 1-butene-1-yl, 1-butene-2-yl, 1-butene-3-yl, 2-butene-1-yl, 2-butene-2-yl, octenyl and decenyl. Non-limiting examples of C₂-C₁₀ alkynyl may include ethynyl, propynyl, butynyl, pentynyl, hexynyl, heptynyl, octynyl, nonynyl and decynyl. Saturated C₁-C₁₀ alkyl, C₂-C₁₀ alkenyl or C₂-C₁₀ alkynyl may be, for example, and without limitation, interrupted by one or more heteroatoms which are independently nitrogen, sulfur or oxygen.

As used herein, cyclic C₃-C₁₀ alkyl may include, for example, and without limitation, saturated C₃-C₁₀ cycloalkyl, C₃-C₁₀ cycloalkenyl, C₃-C₁₀ cycloalkynyl, C₆₋₁₀ aryl, C₆₋₉ aryl-C₁₋₄ alkyl, C₆₋₈ aryl-C₂₋₄ alkenyl, C₆₋₈ aryl-C₂₋₄ alkynyl, a 4- to 10-membered non-aromatic heterocyclic group containing one or more heteroatoms which are independently nitrogen, sulfur or oxygen, and a 5- to 10-membered aromatic heterocyclic group containing one or more heteroatoms which are independently nitrogen, sulfur or oxygen. Non-limiting examples of the saturated C₃-C₁₀ cycloalkyl group may include cyclopropanyl, cyclobutanyl, cyclopentanyl, cyclohexanyl, cycloheptanyl, cyclooctanyl, cyclononanyl and cyclodecanyl. Non-limiting examples of the C₃-C₁₀ cycloalkenyl group may include cyclopropenyl, cyclobutenyl, cyclopentenyl, cyclohexenyl, cycloheptenyl, cyclooctenyl, cyclononanenyl and cyclodecanenyl. Non-limiting examples of the C₆-C₁₀ aryl group may include phenyl (Ph), pentalenyl, indenyl, naphthyl, and azulenyl. The C₆₋₉ aryl-C₁₋₄ alkyl group may be, for example, and without limitation, a C₁₋₄ alkyl group as defined anywhere above having a C₆₋₉ aryl group as defined anywhere above as a substituent. The C₆₋₈ aryl-C₂₋₄ alkenyl group may be, for example, and without limitation, a C₂₋₄ alkenyl as defined anywhere above having a C₆₋₈ aryl group as defined anywhere above as a substituent. The C₆₋₈ aryl-C₂₋₄ alkynyl group may be, for example, and without limitation, a C₂₋₄ alkynyl group as defined anywhere above having a C₆₋₈ aryl group as defined anywhere above as a substituent. Non-limiting examples of the 4- to 10-membered non-aromatic heterocyclic group containing one or more heteroatoms which are independently nitrogen, sulfur or oxygen may include pyrrolidinyl, pyrrolinyl, piperidinyl, piperazinyl, imidazolinyl, pyrazolidinyl, imidazolydinyl, morpholinyl, tetrahydropyranyl, azetidinyl, oxetanyl, oxathiolanyl, phthalimide and succinimide. Non-limiting examples of the 5- to 10-membered aromatic heterocyclic group containing one or more heteroatoms which are independently nitrogen, sulfur or oxygen may include pyrrolyl, pyridinyl, pyridazinyl, pyrimidinyl, pirazinyl, imidazolyl, thiazolyl and oxazolyl.

Non-limiting examples of one to ten carbon substituted or unsubstituted acyl include acetyl, propionyl, butanoyl and pentanoyl. Non-limiting examples of C₁-C₁₀ alkoxy include methoxy, ethoxy, propoxy and butoxy.

As used herein, the symbol

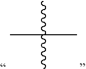

(hereinafter may be referred to as “a point of attachment bond”) denotes a bond that is a point of attachment between two chemical entities, one of which is depicted as being attached to the point of attachment bond and the other of which is not depicted as being attached to the point of attachment bond. For example,

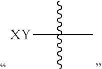

indicates that the chemical entity “XY” is bonded to another chemical entity via the point of attachment bond. Furthermore, the specific point of attachment to the non-depicted chemical entity may be specified by inference. For example The compound CH₃—R³, wherein R³ is H or

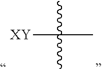

infers that when R³ is “XY”, the point of attachment bond is the same bond as the bond by which R³ is depicted as being bonded to CH₃.

As used herein, the term “moiety” refers to a moiety set out in the following Table 1.

TABLE 1
MOIETIES
Amino Acid Based Moieties

| | |
|---|---|
| 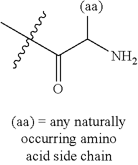 | 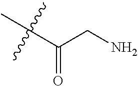 [SMILES: *C(=O)CN] |
| 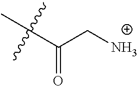 [SMILES: *C(=O)C[NH3+]] | 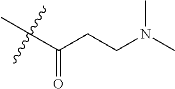 [SMILES: *C(=O)CCN(C)C] |

Polyethylene Glycol Based Moieties

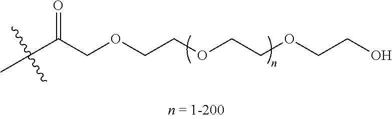

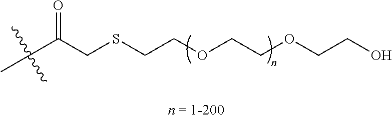

Phosphate Based Moieties

| | |
|---|---|
| 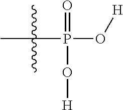 [SMILES: *P(=O)(O)O] | 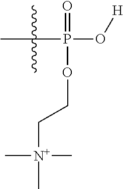 [SMILES: *P(=O)(O)OCC[N+](C)(C)C] |
| 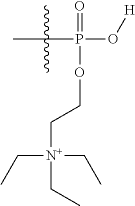 [SMILES: *P(=O)(O)OCC[N+](CC)(CC)CC] | 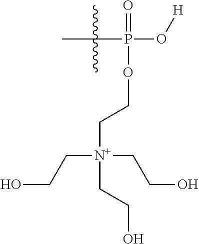 [SMILES: *P(=O)(O)OCC[N+](CCO)(CCO)CCO] |

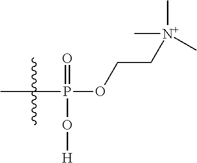 [SMILES: *P(=O)(O)OCC[N+](C)(C)C]

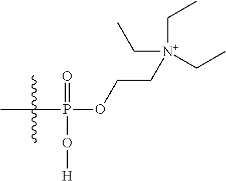 [SMILES: *P(=O)(O)OCC[N+](CC)(CC)CC]

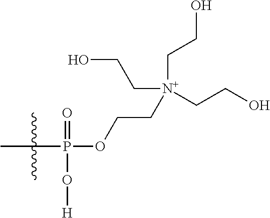 [SMILES: *P(=O)(O)OCC[N+](CCO)(CCO)CCO]

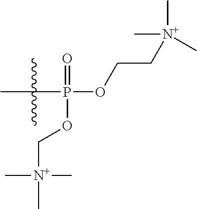

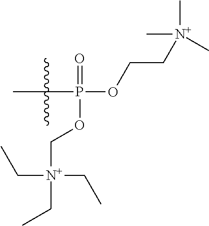 [SMILES: *P(=O)(OCC[N+](C)(C)C)OC[N+](CC)(CC)CC]

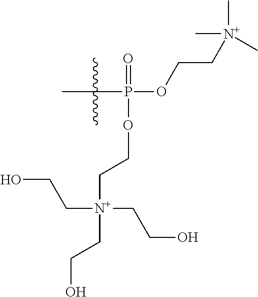 [SMILES: *P(=O)(OCC[N+](C)(C)C)OCC[N+](CCO)(CCO)CCO]

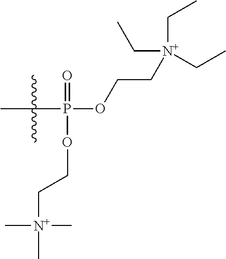 [SMILES: *P(=O)(OCC[N+](C)(C)C)OCC[N+](CC)(CC)CC]

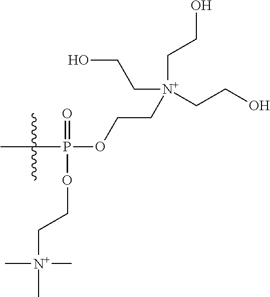 [SMILES: *P(=O)(OCC[N+](C)(C)C)OCC[N+](CCO)(CCO)CCO]

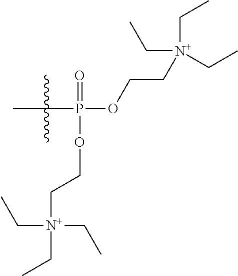 [SMILES: *P(=O)(OCC[N+](CC)(CC)CC)OCC[N+](CC)(CC)CC]

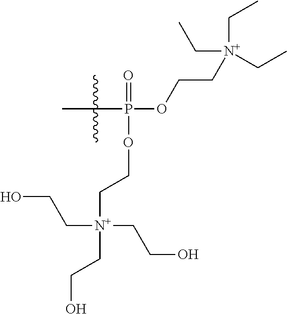 [SMILES: *P(=O)(OCC[N+](CC)(CC)CC)OCC[N+](CCO)(CCO)CCO]

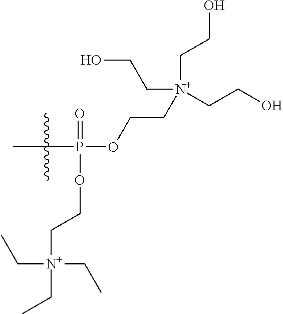 [SMILES: *P(=O)(OCC[N+](CC)(CC)CC)OCC[N+](CCO)(CCO)CCO]

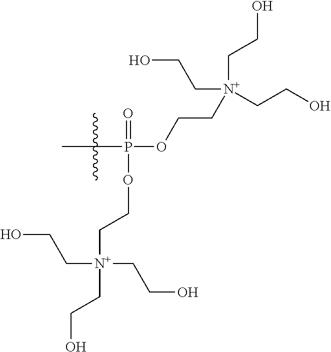 [SMILES: *P(=O)(OCC[N+](CCO)(CCO)CCO)OCC[N+](CCO)(CCO)CCO]

Moieties may be, for example, and without limitation, subdivided into three groups: 1) amino acid based moieties; 2) polyethylene glycol based moieties; and 3) phosphate based moieties. In the Moieties Table 1 above, the first four moieties are amino acid based moieties, the fifth and sixth are polyethylene glycol based moieties and the remaining moieties are phosphate based moieties.

The amino acid side chains of naturally occurring amino acids (as often denoted herein using “(aa)”) are well known to a person of skill in the art and may be found in a variety of text books such as “Molecular Cell Biology” by James Darnell et al. Third Edition, published by Scientific American Books in 1995. Often the naturally occurring amino acids are represented by the formula (NH₂)C(COOH)(H)(R), where the chemical groups in brackets are each bonded to the carbon not in brackets. R represents the side chains in this particular formula.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In other particular embodiments the compounds shown in TABLE 2 are provided. The compounds in TABLE 2 may be made by the methods described herein or by methods known in the art and in some instances have been made and tested (i.e. see TABLE 3), but many have yet to be made and tested.

TABLE 2

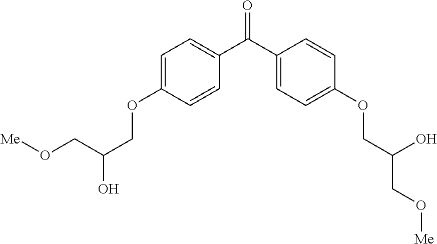 [SMILES: COCC(O)COc1ccc(C(=O)c2ccc(OCC(O)COC)cc2)cc1]

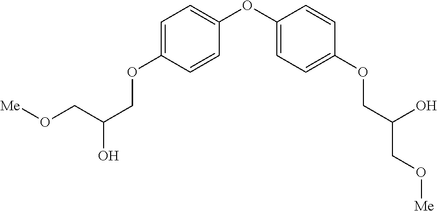 [SMILES: COCC(O)COc1ccc(Oc2ccc(OCC(O)COC)cc2)cc1]

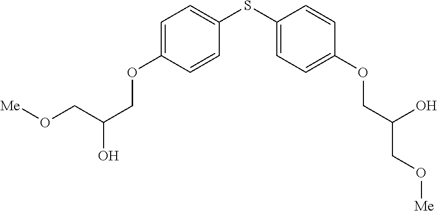 [SMILES: COCC(O)COc1ccc(Sc2ccc(OCC(O)COC)cc2)cc1]

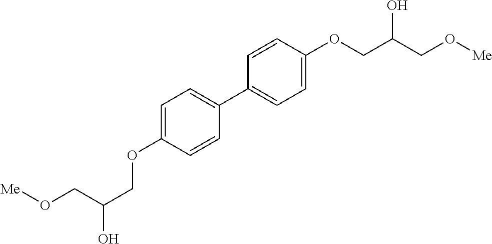 [SMILES: COCC(O)COc1ccc(-c2ccc(OCC(O)COC)cc2)cc1]

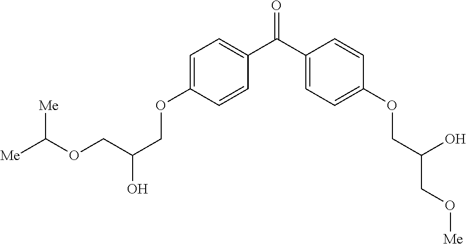 [SMILES: COCC(O)COc1ccc(C(=O)c2ccc(OCC(O)COC(C)C)cc2)cc1]

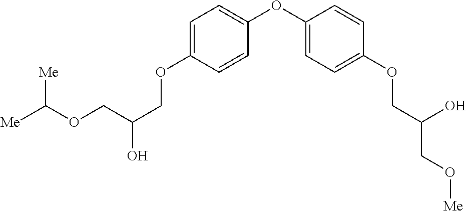 [SMILES: COCC(O)COc1ccc(Oc2ccc(OCC(O)COC(C)C)cc2)cc1]

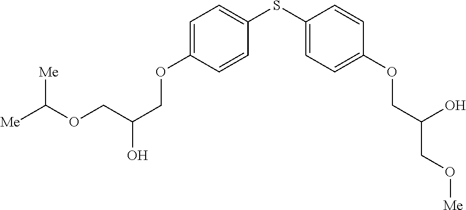 [SMILES: COCC(O)COc1ccc(Sc2ccc(OCC(O)COC(C)C)cc2)cc1]

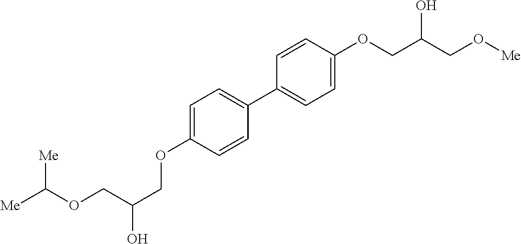 [SMILES: COCC(O)COc1ccc(-c2ccc(OCC(O)COC(C)C)cc2)cc1]

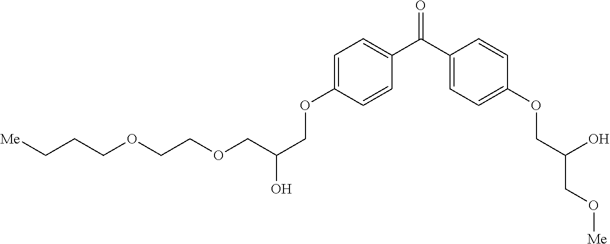 [SMILES: CCCCOCCOCC(O)COc1ccc(C(=O)c2ccc(OCC(O)COC)cc2)cc1]

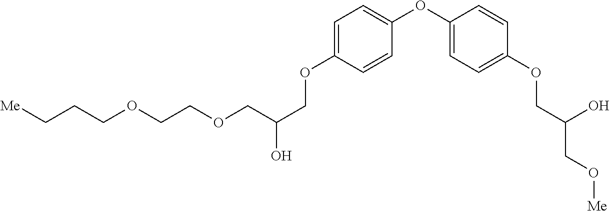 [SMILES: CCCCOCCOCC(O)COc1ccc(Oc2ccc(OCC(O)COC)cc2)cc1]

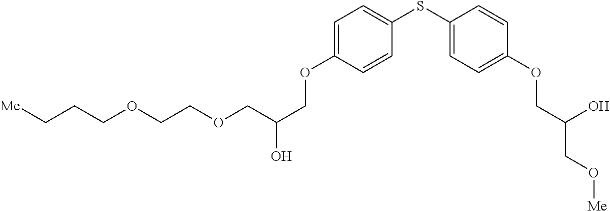 [SMILES: CCCCOCCOCC(O)COc1ccc(Sc2ccc(OCC(O)COC)cc2)cc1]

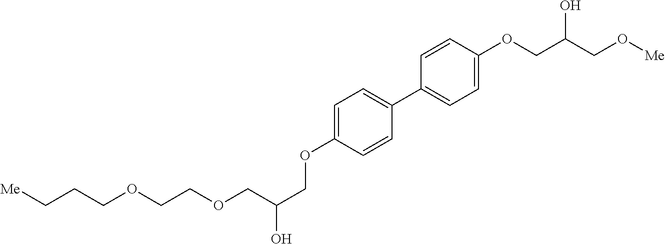 [SMILES: CCCCOCCOCC(O)COc1ccc(-c2ccc(OCC(O)COC)cc2)cc1]

| | |
|---|---|
| 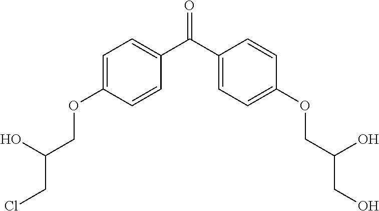 [SMILES: O=C(c1ccc(OCC(O)CO)cc1)c1ccc(OCC(O)CCl)cc1] | 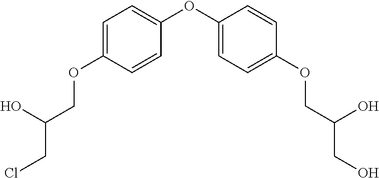 [SMILES: OCC(O)COc1ccc(Oc2ccc(OCC(O)CCl)cc2)cc1] |
| 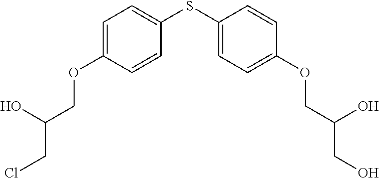 [SMILES: OCC(O)COc1ccc(Sc2ccc(OCC(O)CCl)cc2)cc1] | 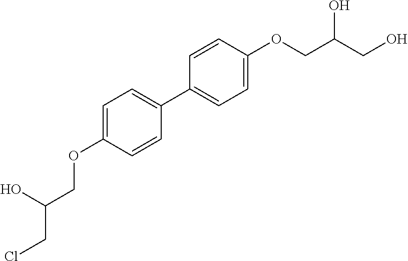 [SMILES: OCC(O)COc1ccc(-c2ccc(OCC(O)CCl)cc2)cc1] |

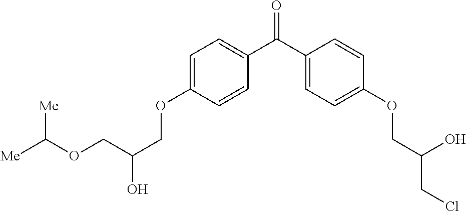 [SMILES: CC(C)OCC(O)COc1ccc(C(=O)c2ccc(OCC(O)CCl)cc2)cc1]

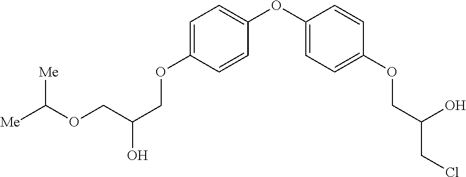 [SMILES: CC(C)OCC(O)COc1ccc(Oc2ccc(OCC(O)CCl)cc2)cc1]

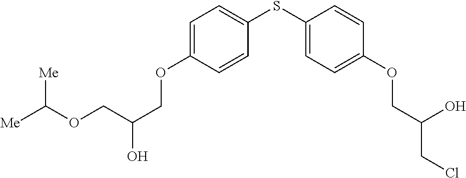 [SMILES: CC(C)OCC(O)COc1ccc(Sc2ccc(OCC(O)CCl)cc2)cc1]

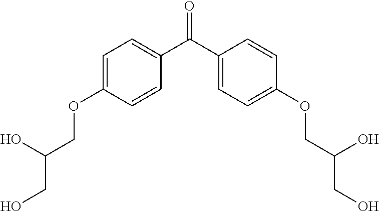 [SMILES: O=C(c1ccc(OCC(O)CO)cc1)c1ccc(OCC(O)CO)cc1]

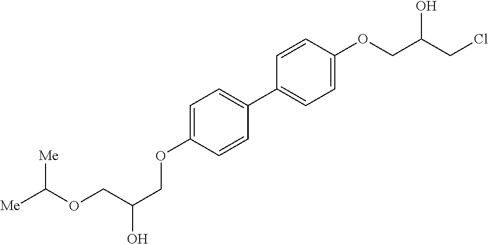 [SMILES: CC(C)OCC(O)COc1ccc(-c2ccc(OCC(O)CCl)cc2)cc1]

| | |
|---|---|
| 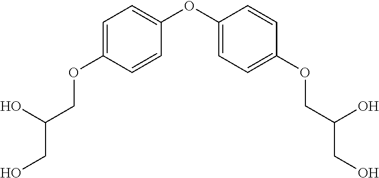 [SMILES: OCC(O)COc1ccc(Oc2ccc(OCC(O)CO)cc2)cc1] | 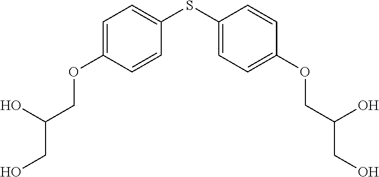 [SMILES: OCC(O)COc1ccc(Sc2ccc(OCC(O)CO)cc2)cc1] |

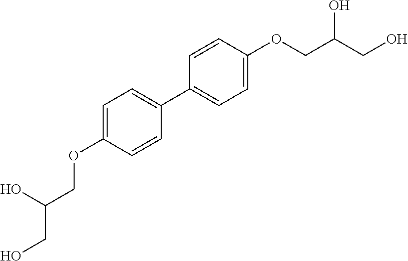 [SMILES: OCC(O)COc1ccc(-c2ccc(OCC(O)CO)cc2)cc1]

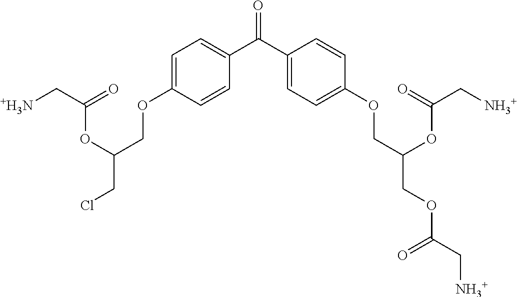 [SMILES: [NH3+]CC(=O)OCC(COc1ccc(C(=O)c2ccc(OCC(CCl)OC(=O)C[NH3+])cc2)cc1)OC(=O)C[NH3+]]

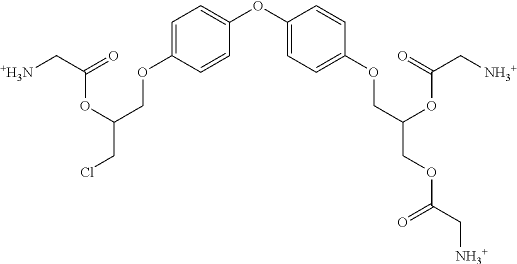 [SMILES: [NH3+]CC(=O)OCC(COc1ccc(Oc2ccc(OCC(CCl)OC(=O)C[NH3+])cc2)cc1)OC(=O)C[NH3+]]

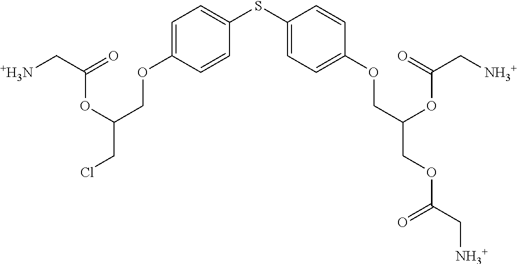 [SMILES: [NH3+]CC(=O)OCC(COc1ccc(Sc2ccc(OCC(CCl)OC(=O)C[NH3+])cc2)cc1)OC(=O)C[NH3+]]

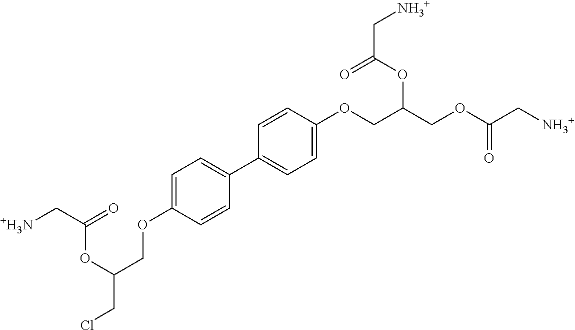 [SMILES: [NH3+]CC(=O)OCC(COc1ccc(-c2ccc(OCC(CCl)OC(=O)C[NH3+])cc2)cc1)OC(=O)C[NH3+]]

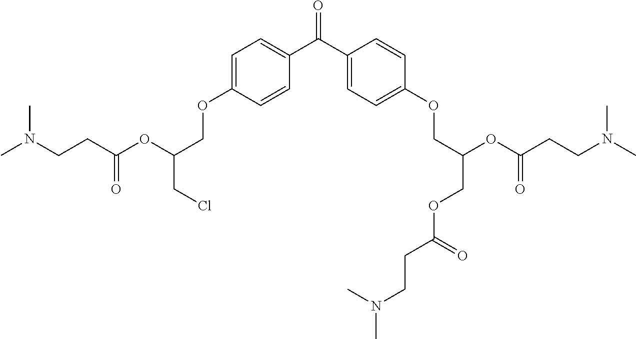

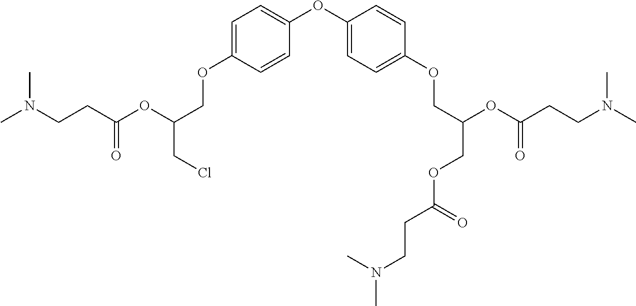

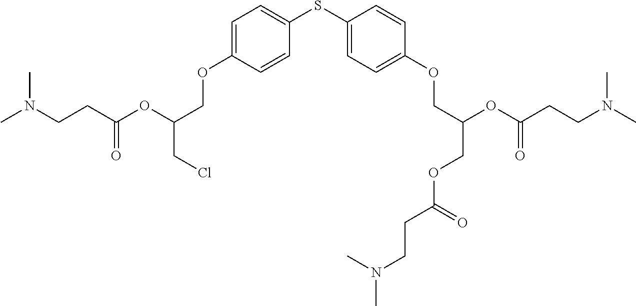

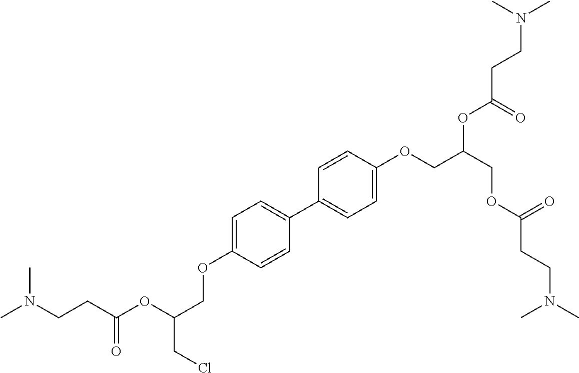

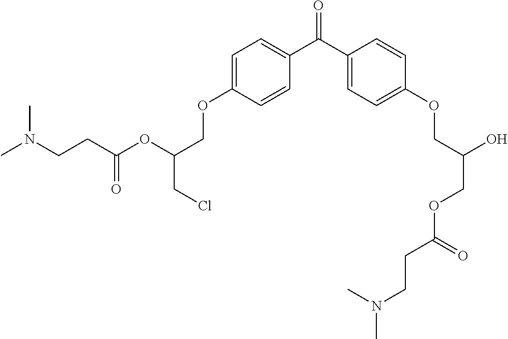 [SMILES: CN(C)CCC(=O)OCC(O)COc1ccc(C(=O)c2ccc(OCC(CCl)OC(=O)CCN(C)C)cc2)cc1]

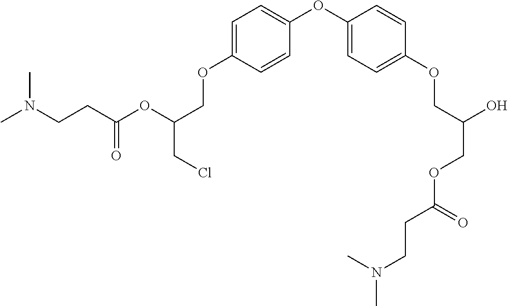 [SMILES: CN(C)CCC(=O)OCC(O)COc1ccc(Oc2ccc(OCC(CCl)OC(=O)CCN(C)C)cc2)cc1]

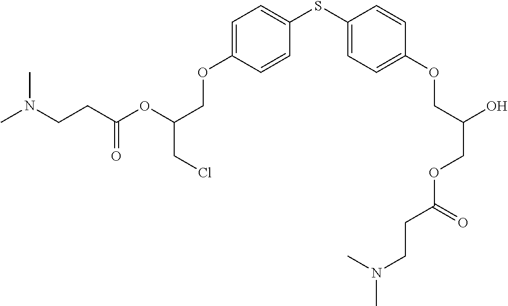 [SMILES: CN(C)CCC(=O)OCC(O)COc1ccc(Sc2ccc(OCC(CCl)OC(=O)CCN(C)C)cc2)cc1]

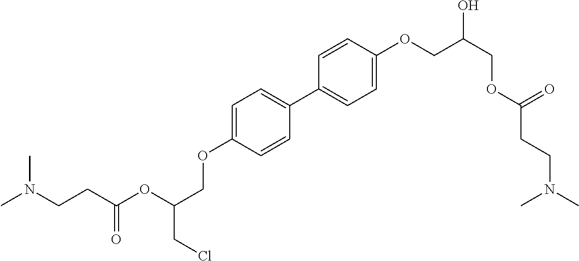

| | |
|---|---|
| 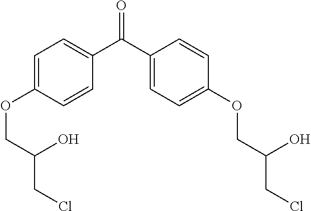 [SMILES: O=C(c1ccc(OCC(O)CCl)cc1)c1ccc(OCC(O)CCl)cc1] | 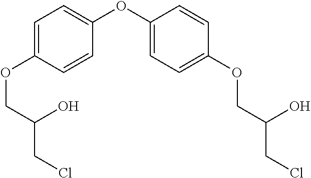 [SMILES: OC(CCl)COc1ccc(Oc2ccc(OCC(O)CCl)cc2)cc1] |
| 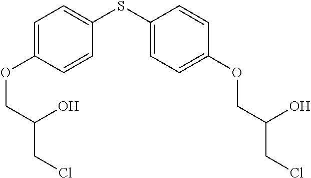 [SMILES: OC(CCl)COc1ccc(Sc2ccc(OCC(O)CCl)cc2)cc1] | 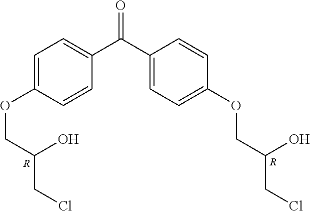 |
| 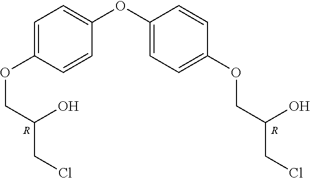 | 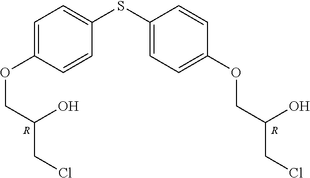 |
| 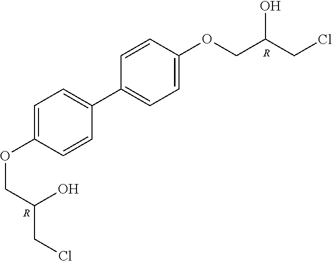 | 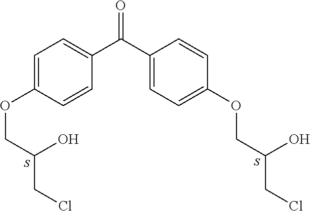 |
| 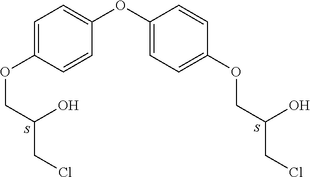 | 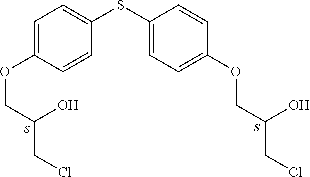 |
| 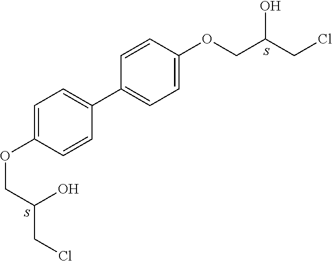 | 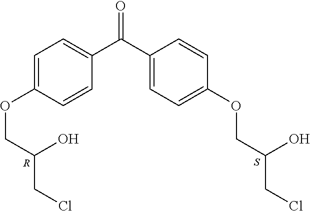 |
| 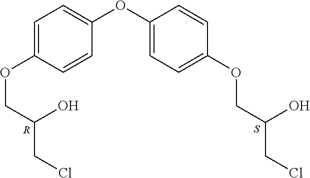 | 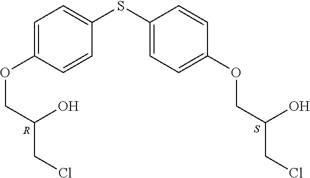 |
| 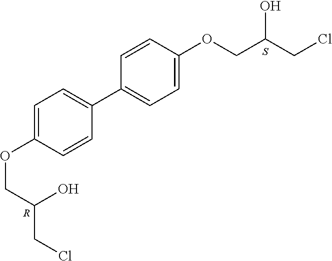 | 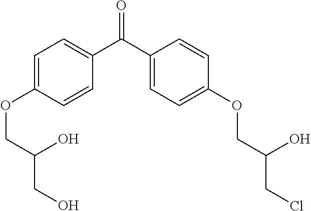 [SMILES: O=C(c1ccc(OCC(O)CO)cc1)c1ccc(OCC(O)CCl)cc1] |
| 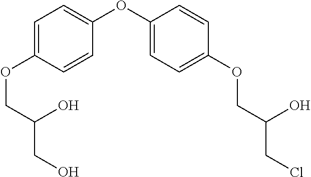 [SMILES: OCC(O)COc1ccc(Oc2ccc(OCC(O)CCl)cc2)cc1] | 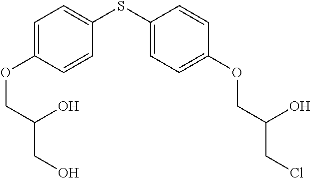 [SMILES: OCC(O)COc1ccc(Sc2ccc(OCC(O)CCl)cc2)cc1] |
| 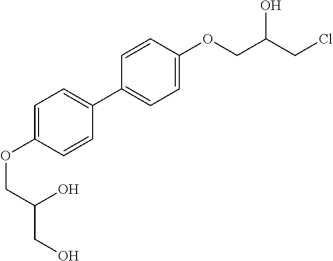 [SMILES: OCC(O)COc1ccc(-c2ccc(OCC(O)CCl)cc2)cc1] | 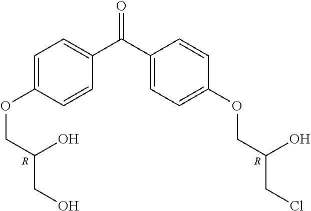 |
| 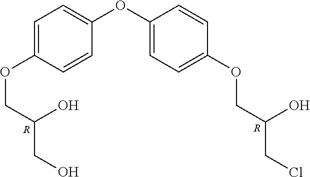 | 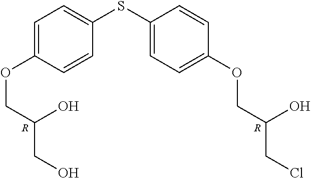 |
| 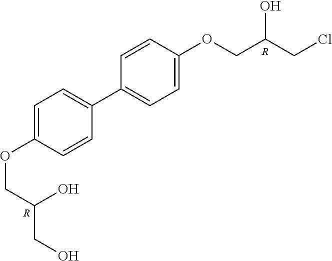 | 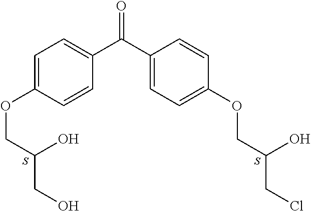 |
| 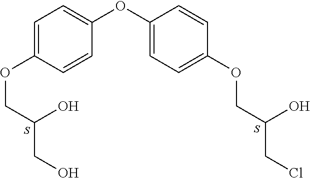 | 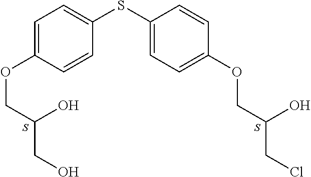 |
| 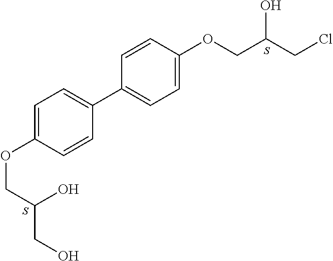 | 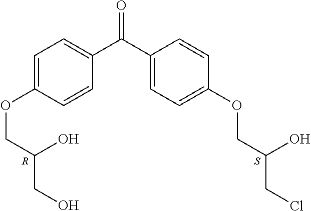 |
| 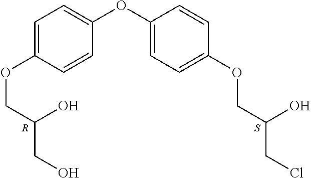 | 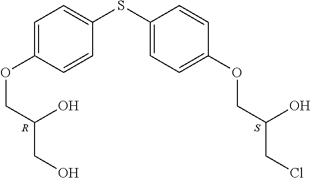 |
| 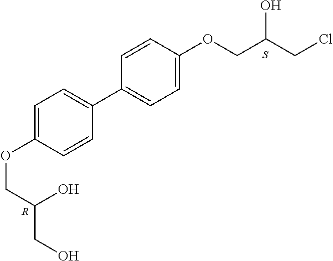 | 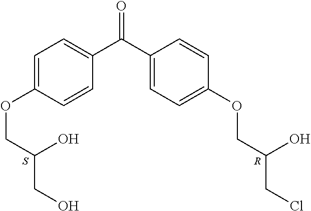 |
| 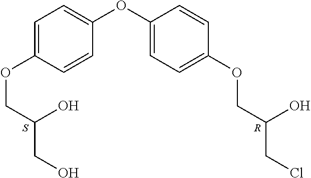 | 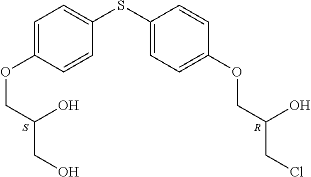 |
| 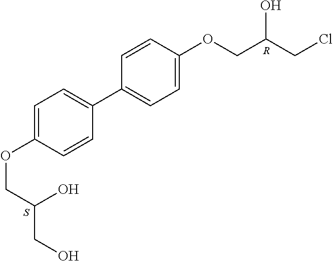 | 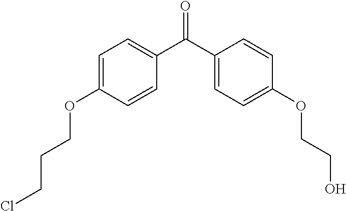 [SMILES: O=C(c1ccc(OCCO)cc1)c1ccc(OCCCCl)cc1] |
| 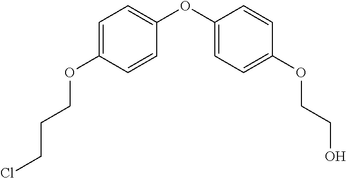 [SMILES: OCCOc1ccc(Oc2ccc(OCCCCl)cc2)cc1] | 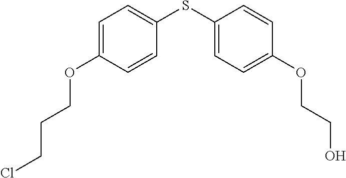 [SMILES: OCCOc1ccc(Sc2ccc(OCCCCl)cc2)cc1] |
| 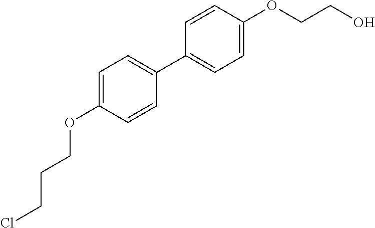 [SMILES: OCCOc1ccc(-c2ccc(OCCCCl)cc2)cc1] | 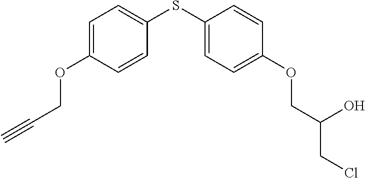 [SMILES: C#CCOc1ccc(Sc2ccc(OCC(O)CCl)cc2)cc1] |

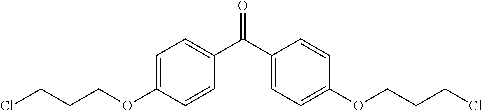 [SMILES: O=C(c1ccc(OCCCCl)cc1)c1ccc(OCCCCl)cc1]

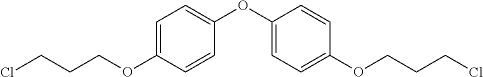 [SMILES: ClCCCOc1ccc(Oc2ccc(OCCCCl)cc2)cc1]

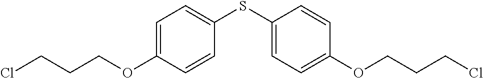 [SMILES: ClCCCOc1ccc(Sc2ccc(OCCCCl)cc2)cc1]

| | |
|---|---|
| 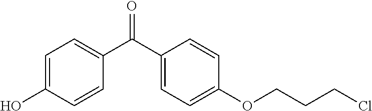 [SMILES: O=C(c1ccc(O)cc1)c1ccc(OCCCCl)cc1] | 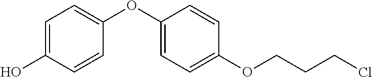 [SMILES: Oc1ccc(Oc2ccc(OCCCCl)cc2)cc1] |
| 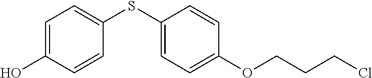 [SMILES: Oc1ccc(Sc2ccc(OCCCCl)cc2)cc1] | 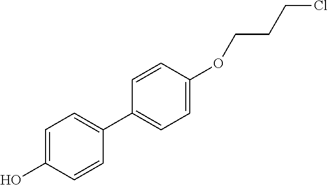 [SMILES: Oc1ccc(-c2ccc(OCCCCl)cc2)cc1] |

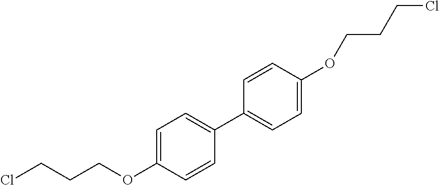 [SMILES: ClCCCOc1ccc(-c2ccc(OCCCCl)cc2)cc1]

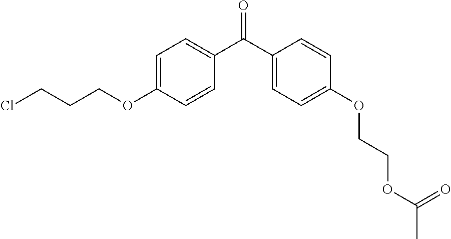 [SMILES: CC(=O)OCCOc1ccc(C(=O)c2ccc(OCCCCl)cc2)cc1]

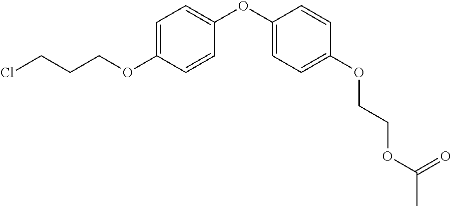 [SMILES: CC(=O)OCCOc1ccc(Oc2ccc(OCCCCl)cc2)cc1]

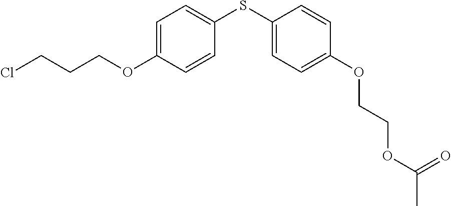 [SMILES: CC(=O)OCCOc1ccc(Sc2ccc(OCCCCl)cc2)cc1]

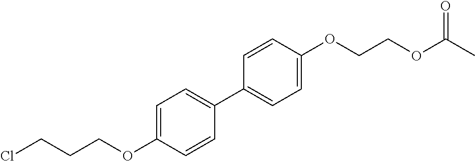 [SMILES: CC(=O)OCCOc1ccc(-c2ccc(OCCCCl)cc2)cc1]

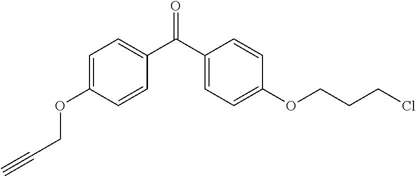 [SMILES: C#CCOc1ccc(C(=O)c2ccc(OCCCCl)cc2)cc1]

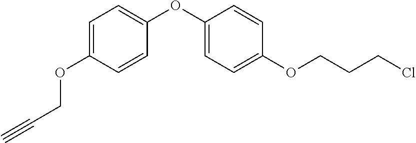 [SMILES: C#CCOc1ccc(Oc2ccc(OCCCCl)cc2)cc1]

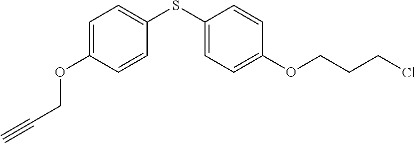 [SMILES: C#CCOc1ccc(Sc2ccc(OCCCCl)cc2)cc1]

| | |
|---|---|
| 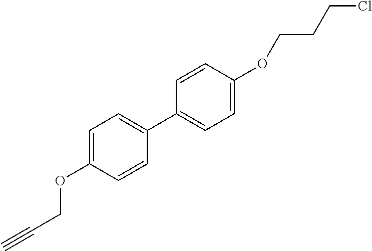 [SMILES: C#CCOc1ccc(-c2ccc(OCCCCl)cc2)cc1] | 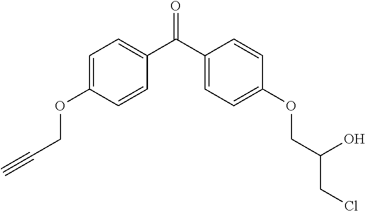 [SMILES: C#CCOc1ccc(C(=O)c2ccc(OCC(O)CCl)cc2)cc1] |

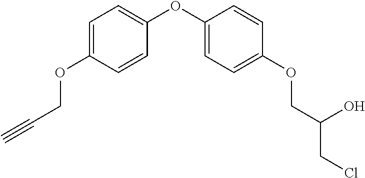 [SMILES: C#CCOc1ccc(Oc2ccc(OCC(O)CCl)cc2)cc1]

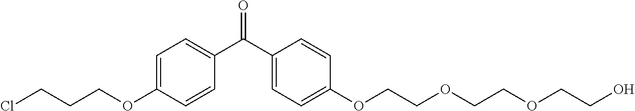 [SMILES: O=C(c1ccc(OCCCCl)cc1)c1ccc(OCCOCCOCCO)cc1]

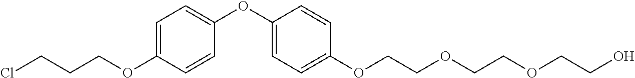 [SMILES: OCCOCCOCCOc1ccc(Oc2ccc(OCCCCl)cc2)cc1]

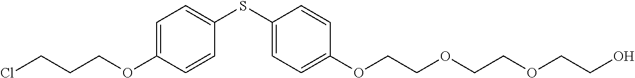 [SMILES: OCCOCCOCCOc1ccc(Sc2ccc(OCCCCl)cc2)cc1]

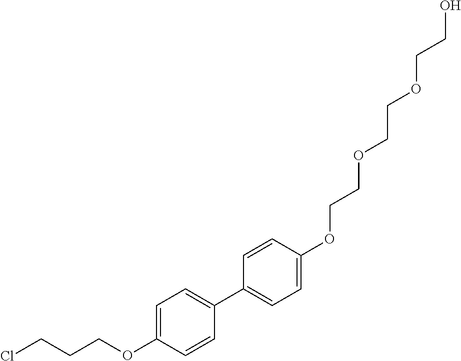 [SMILES: OCCOCCOCCOc1ccc(-c2ccc(OCCCCl)cc2)cc1]

| | |
|---|---|
| 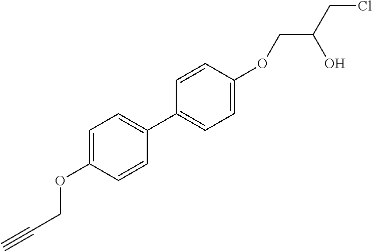 [SMILES: C#CCOc1ccc(-c2ccc(OCC(O)CCl)cc2)cc1] | 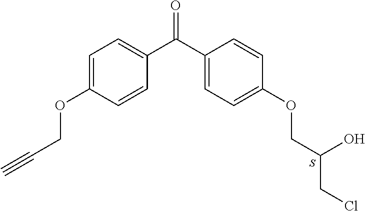 |
| 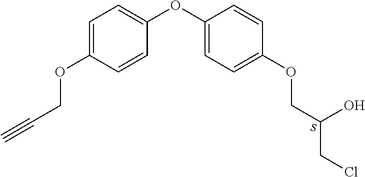 | 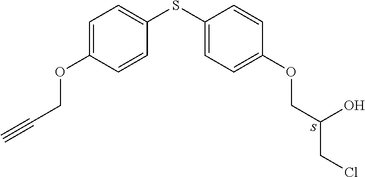 |
| 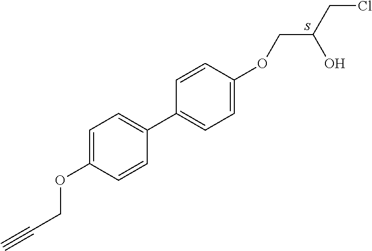 | 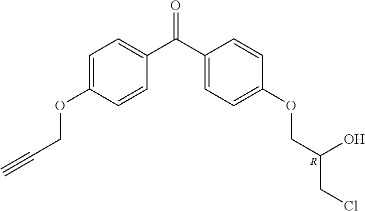 |
| 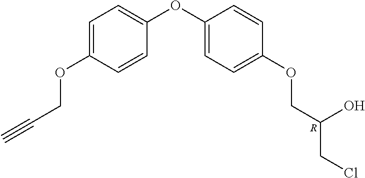 | 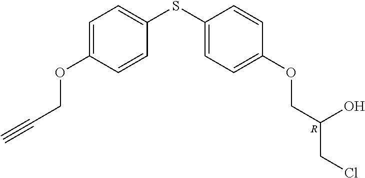 |
| 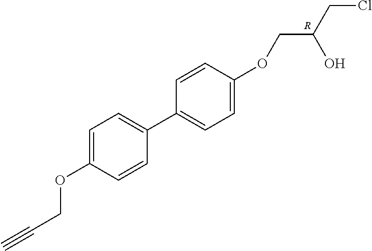 | 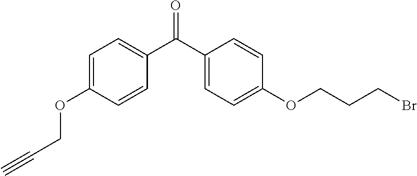 [SMILES: C#CCOc1ccc(C(=O)c2ccc(OCCCBr)cc2)cc1] |
| 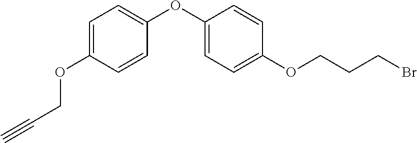 [SMILES: C#CCOc1ccc(Oc2ccc(OCCCBr)cc2)cc1] | 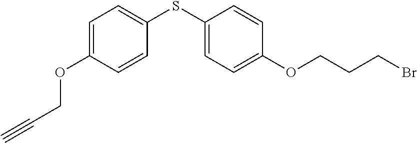 [SMILES: C#CCOc1ccc(Sc2ccc(OCCCBr)cc2)cc1] |
| 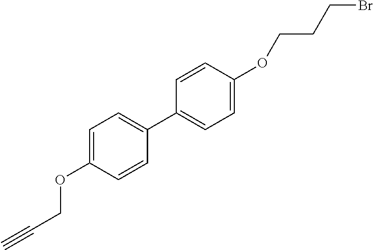 [SMILES: C#CCOc1ccc(-c2ccc(OCCCBr)cc2)cc1] | 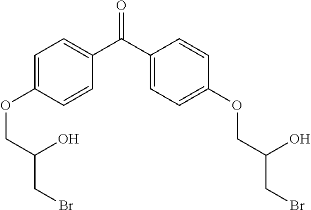 [SMILES: O=C(c1ccc(OCC(O)CBr)cc1)c1ccc(OCC(O)CBr)cc1] |
| 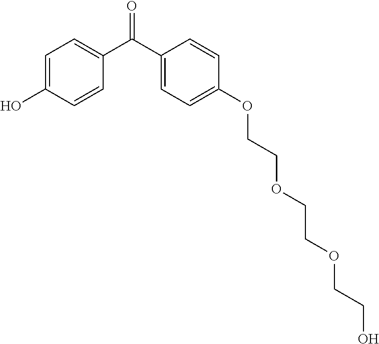 [SMILES: O=C(c1ccc(O)cc1)c1ccc(OCCOCCOCCO)cc1] | 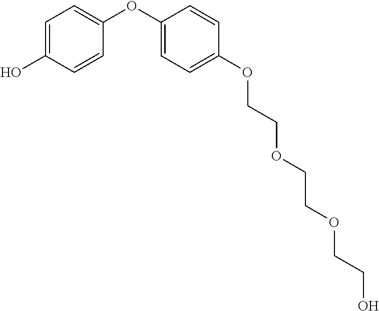 [SMILES: OCCOCCOCCOc1ccc(Oc2ccc(O)cc2)cc1] |
| 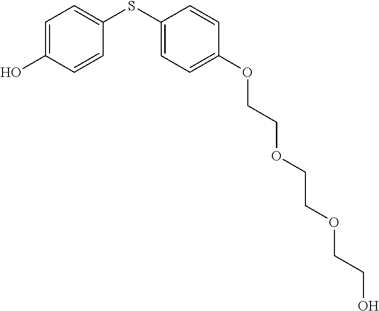 [SMILES: OCCOCCOCCOc1ccc(Sc2ccc(O)cc2)cc1] | 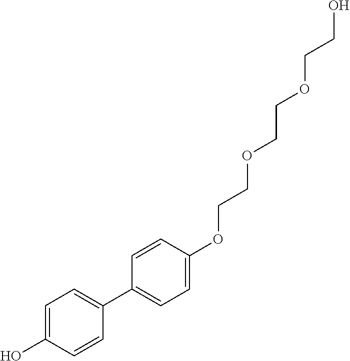 [SMILES: OCCOCCOCCOc1ccc(-c2ccc(O)cc2)cc1] |
| 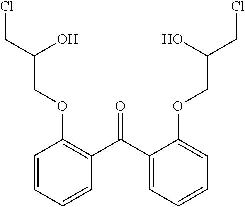 [SMILES: O=C(c1ccccc1OCC(O)CCl)c1ccccc1OCC(O)CCl] | 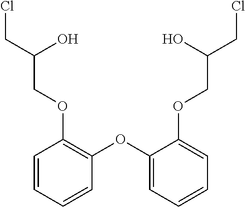 [SMILES: OC(CCl)COc1ccccc1Oc1ccccc1OCC(O)CCl] |
| 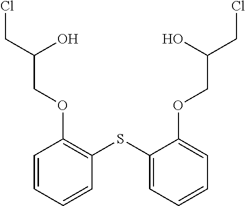 [SMILES: OC(CCl)COc1ccccc1Sc1ccccc1OCC(O)CCl] | 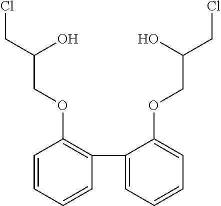 [SMILES: OC(CCl)COc1ccccc1-c1ccccc1OCC(O)CCl] |
| 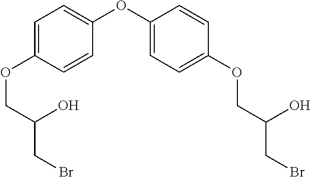 [SMILES: OC(CBr)COc1ccc(Oc2ccc(OCC(O)CBr)cc2)cc1] | 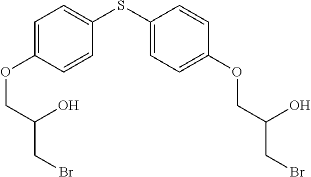 [SMILES: OC(CBr)COc1ccc(Sc2ccc(OCC(O)CBr)cc2)cc1] |
| 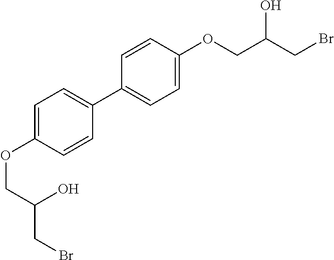 [SMILES: OC(CBr)COc1ccc(-c2ccc(OCC(O)CBr)cc2)cc1] | 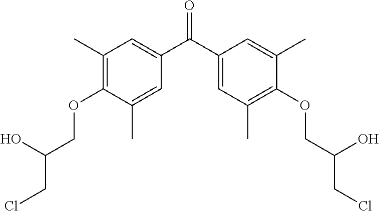 [SMILES: Cc1cc(C(=O)c2cc(C)c(OCC(O)CCl)c(C)c2)cc(C)c1OCC(O)CCl] |
| 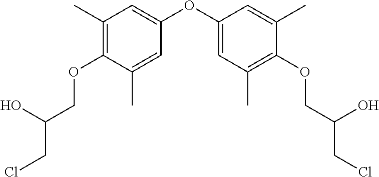 [SMILES: Cc1cc(Oc2cc(C)c(OCC(O)CCl)c(C)c2)cc(C)c1OCC(O)CCl] | 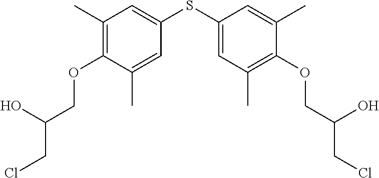 [SMILES: Cc1cc(Sc2cc(C)c(OCC(O)CCl)c(C)c2)cc(C)c1OCC(O)CCl] |

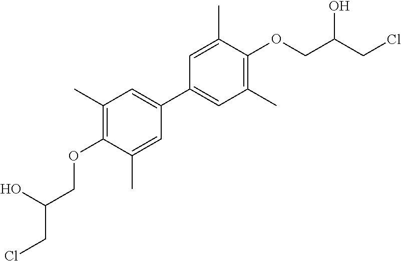 [SMILES: Cc1cc(-c2cc(C)c(OCC(O)CCl)c(C)c2)cc(C)c1OCC(O)CCl]

| | |
|---|---|
| 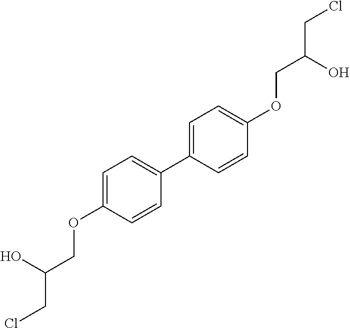 [SMILES: OC(CCl)COc1ccc(-c2ccc(OCC(O)CCl)cc2)cc1] | 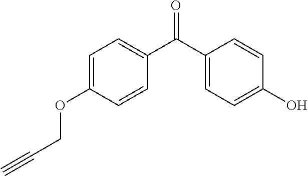 [SMILES: C#CCOc1ccc(C(=O)c2ccc(O)cc2)cc1] |
| 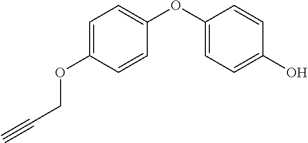 [SMILES: C#CCOc1ccc(Oc2ccc(O)cc2)cc1] | 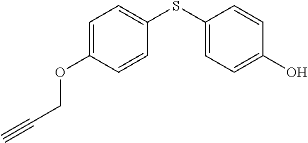 [SMILES: C#CCOc1ccc(Sc2ccc(O)cc2)cc1] |
| 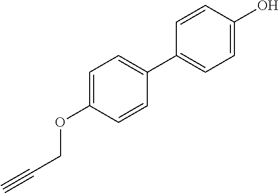 [SMILES: C#CCOc1ccc(-c2ccc(O)cc2)cc1] | 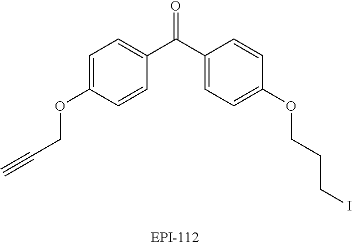 [SMILES: C#CCOc1ccc(C(=O)c2ccc(OCCCI)cc2)cc1] |

In some embodiments, the compounds as described herein or acceptable salts thereof above may be used for systemic treatment of at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. In some embodiments, the compounds as described herein or acceptable salts thereof above may be used in the preparation of a medicament or a composition for systemic treatment of an indication described herein. In some embodiments, methods of systemically treating any of the indications described herein are also provided. Some aspects of this invention, make use of compositions comprising a compound described herein and a pharmaceutically acceptable excipients or carrier. In some embodiments, the prostate cancer is androgen-independent prostate cancer (also referred to as hormone refractory, castration resistant, androgen deprivation resistant, androgen ablation resistant, androgen depletion-independent, castration-recurrent, anti-androgen-recurrent). In some embodiments the prostate cancer is androgen-dependent or androgen-sensitive. Methods of treating any of the indications described herein are also provided. Such methods may include administering a compound as described herein or a composition of a compound as described herein, or an effective amount of a compound as described herein or composition of a compound as described herein to a subject in need thereof.

Compounds as described herein may be in the free form or in the form of a salt thereof. In some embodiments, compounds as described herein may be in the form of a pharmaceutically acceptable salt, which are known in the art (Berge et al., I Pharm. Sci. 1977, 66, 1). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

In some embodiments, pharmaceutical compositions in accordance with this invention may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen-independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. For example, compounds and all their different forms as described herein may be used as neoadjuvant (prior), adjunctive (during), and/or adjuvant (after) therapy with surgery, radiation (brachytherapy or external beam), or other therapies (eg. HIFU).

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions. Some compounds of this invention may be toxic at some concentrations. Titration studies may be used to determine toxic and non-toxic concentrations. Toxicity may be evaluated by examining a particular compound's or composition's specificity across cell lines using PC3 cells as a negative control that do not express AR. Animal studies may be used to provide an indication if the compound has any effects on other tissues. Systemic therapy that targets the AR will not likely cause major problems to other tissues since antiandrogens and androgen insensitivity syndrome are not fatal.

Compounds as described herein may be administered to a subject. As used herein, a “subject” may be a human, non-human primate, rat, mouse, cow, horse, pig, sheep, goat, dog, cat, etc. The subject may be suspected of having or at risk for having a cancer, such as prostate cancer, breast cancer, ovarian cancer or endometrial cancer, or suspected of having or at risk for having acne, hirsutism, alopecia, benign prostatic hyperplasia, ovarian cysts, polycystic ovary disease, precocious puberty, or age-related macular degeneration. Diagnostic methods for various cancers, such as prostate cancer, breast cancer, ovarian cancer or endometrial cancer, and diagnostic methods for acne, hirsutism, alopecia, benign prostatic hyperplasia, ovarian cysts, polycystic ovary disease, precocious puberty, or age-related macular degeneration and the clinical delineation of cancer, such as prostate cancer, breast cancer, ovarian cancer or endometrial cancer, diagnoses and the clinical delineation of acne, hirsutism, alopecia, benign prostatic hyperplasia, ovarian cysts, polycystic ovary disease, precocious puberty, or age-related macular degeneration are known to those of ordinary skill in the art.

Compounds described herein may be used for treatment of at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. Compounds described herein may be used for treatment of prostate cancer. Compounds described herein may be used for treatment of androgen-independent prostate cancer. Compounds described herein may be used for treatment of androgen-dependent prostate cancer. Compounds described herein may be used for preparation of a medicament for treatment of at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. Compounds described herein may be used for the preparation of a medicament for treatment of prostate cancer. Compounds described herein may be used for the preparation of a medicament for treatment of androgen-independent prostate cancer. Compounds described herein may be used for the preparation of a medicament for treatment of androgen-dependent prostate cancer. Compounds described herein may be used in a method for treatment of at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. The method may comprise administering to a subject in need thereof an effective amount of a compound described herein. Compounds described herein may be used in a method of treatment of prostate cancer, the method comprising administering to a subject in need thereof an effective amount of a compound described herein. Compounds described herein may be used in a method of treatment of androgen-independent prostate cancer, the method comprising administering to a subject in need thereof an effective amount of a compound described herein. Compounds described herein may be used in a method of treatment of androgen-dependent prostate cancer, the method comprising administering to a subject in need thereof an effective amount of a compound described herein.

Compounds described herein may also be used in assays and for research purposes. Definitions used include ligand-dependent activation of the androgen receptor (AR) by androgens such as dihydrotestosterone (DHT) or the synthetic androgen (R1881) used for research purposes. Ligand-independent activation of the AR refers to transactivation of the AR in the absence of androgen (ligand) by, for example, stimulation of the cAMP-dependent protein kinase (PKA) pathway with forskolin (FSK). Some compounds and compositions of this invention may inhibit both FSK and androgen (e.g. R1881) induction of ARE-luciferase (ARE-luc). Such compounds may block a mechanism that is common to both ligand-dependent and ligand-independent activation of the AR. This could involve any step in activation of the AR including dissociation of heatshock proteins, essential posttranslational modifications (e.g., acetylation, phosphorylation), nuclear translocation, protein-protein interactions, formation of the transcriptional complex, release of co-repressors, and/or increased degradation. Some compounds and compositions of this invention may inhibit R1881 only and may interfere with a mechanism specific to ligand-dependent activation (e.g., accessibility of the ligand binding domain (LBD) to androgen). Numerous disorders in addition to prostate cancer involve the androgen axis (e.g., acne, hirsutism, alopecia, benign prostatic hyperplasia) and compounds interfering with this mechanism may be used to treat such conditions. Some compounds and compositions of this invention may only inhibit FSK induction and may be specific inhibitors to ligand-independent activation of the AR. These compounds and compositions may interfere with the cascade of events that normally occur with FSK and/or PKA activity or any downstream effects that may play a role on the AR (e.g. FSK increases MAPK activity which has a potent effect on AR activity). Examples may include an inhibitor of cAMP and or PKA or other kinases. Some compounds and compositions of this invention may induce basal levels of activity of the AR (no androgen or stimulation of the PKA pathway). Some compounds and compositions of this invention may increase induction by R1881 or FSK. Such compounds and compositions may stimulate transcription or transactivation of the AR. Some compounds and compositions of this invention may inhibit activity of the androgen receptor. Interleukin-6 (IL-6) also causes ligand-independent activation of the AR in LNCaP cells and can be used in addition to FSK.

Compounds for use in the present invention may be obtained from medical sources or modified using known methodologies from naturally occurring compounds. In addition, methods of preparing or synthesizing compounds of the present invention will be understood by a person of skill in the art having reference to known chemical synthesis principles. For example, Auzou et al 1974European Journal of Medicinal Chemistry 9(5), 548-554 describes suitable synthetic procedures that may be considered and suitably adapted for preparing compounds of any one of the Formula I to XXI as set out above. Other references that may be helpful include: Debasish Das, Jyh-Fu Lee and Soofin Cheng “Sulfonic acid functionalized mesoporous MCM-41 silica as a convenient catalyst for Bisphenol-A synthesis” Chemical Communications, (2001) 2178-2179; U.S. Pat. No. 2,571,217 Davis, Orris L.; Knight, Horace S.; Skinner, John R. (Shell Development Co.) “Halohydrin ethers of phenols.” (1951); and Rokicki, G.; Pawlicki, J.; Kuran, W. “Reactions of 4-chloromethyl-1,3-dioxolan-2-one with phenols as a new route to polyols and cyclic carbonates.” Journal fuer Praktische Chemie (Leipzig) (1985) 327, 718-722.

For example, compounds of the present invention which contain an ether moiety may be obtained with reference to the following general chemical synthetic scheme I:

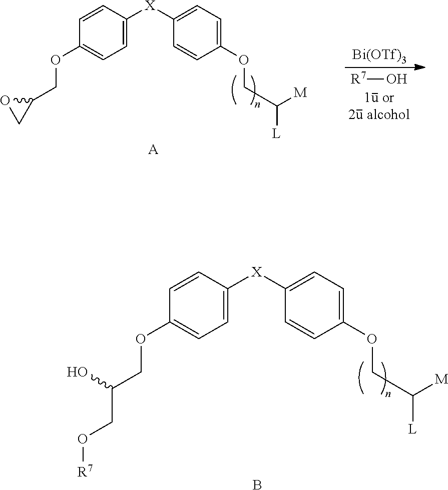

wherein R⁷—OH represents an alcohol and X, M, L, and n are as defined anywhere herein. Bismuth triflate may be added in portions to a solution of racemic derivative A in an alcohol R⁷—OH over the course of the reaction. The mixture may be stirred under suitable conditions (for example, rt for 24 h). The resulting suspension may be quenched by a suitable reagent (for example, by addition of sodium bicarbonate), extracted (for example, with ethyl acetate), dried (for example, over anhydrous magnesium sulphate), and concentrated (for example, under vacuum). The resulting residue may be purified by a suitable method (for example, flash column chromatography on silica gel-eluent: 90% hexane in ethyl acetate) to provide B. A person of skill in the art will understand that the above general scheme I may be suitably adapted to prepare compounds of the present invention which contain a propargyl ether moiety, for example, based on the following general chemical synthetic scheme II:

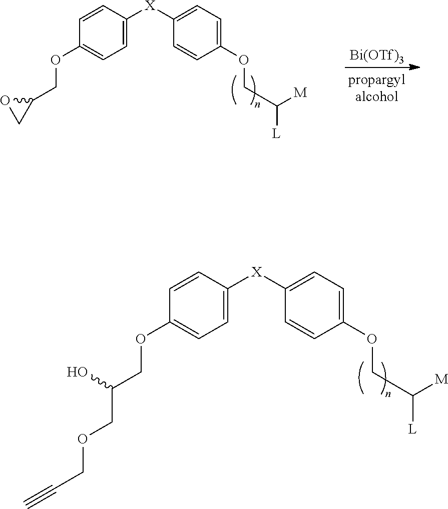

wherein X, M, L, and n are as defined anywhere herein. The general scheme I may be suitably adapted to prepare compounds of the present invention which contain an isopropyl ether moiety, for example, based on the following general chemical synthetic scheme III:

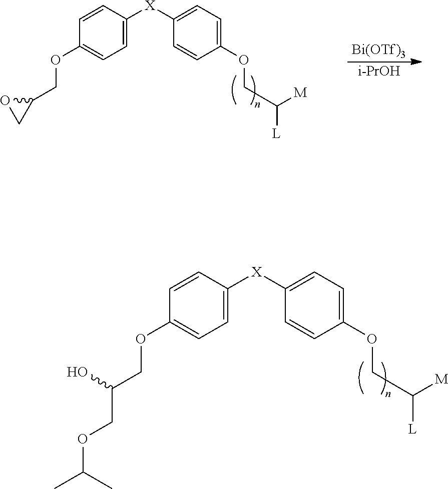

wherein X, M, L, and n are as defined anywhere herein. The general scheme I may be suitably adapted to prepare compounds of the present invention which contain an n-butyl ether moiety, for example, based on the following general chemical synthetic scheme IV:

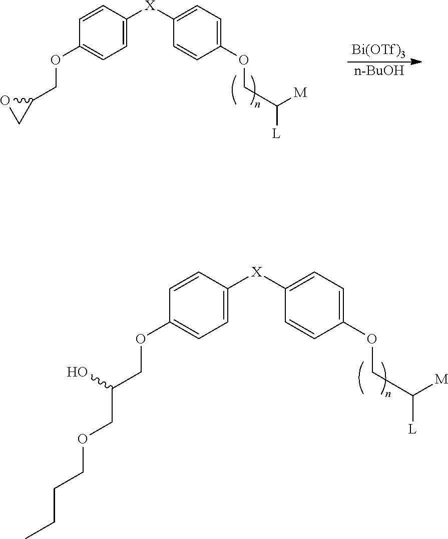

wherein X, M, L, and n are as defined anywhere herein. The general scheme I may be suitably adapted to prepare compounds of the present invention which contain a cyclohexyl ether moiety, for example, based on the following general chemical synthetic scheme V:

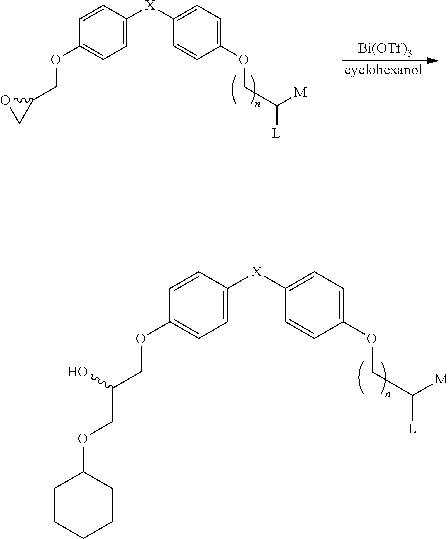

wherein X, M, L, and n are as defined anywhere herein.

General methodologies for chemical preparation of compounds of any one of the Formula I to XXI are described in the following non-limiting exemplary schemes.

Various alternative embodiments and examples of the invention are described herein. These embodiments and examples are illustrative and should not be construed as limiting the scope of the invention.

### EXAMPLES

### General Methodologies

### Chemical Synthesis

All reactions were performed in flame-dried round bottomed flasks. The flasks were fitted with rubber septa and reactions were conducted under a positive pressure of argon unless otherwise specified. Stainless steel syringes were used to transfer air- and moisture-sensitive liquids. Flash column chromatography was performed as described by Still et al. (Still, W. C., Kahn, M., Mitra, A., J. Org. Chem. 1978, 43, 2923) using 230-400 mesh silica gel. Thin-layer chromatography was performed using aluminium plates pre-coated with 0.25 mm 230-400 mesh silica gel impregnated with a fluorescent indicator (254 nm). Thin-layer chromatography plates were visualized by exposure to ultraviolet light and a solution of p-anisaldehyde (1% p-anisaldehyde, 2% H₂SO₄, 20% acetic acid and 77% ethanol) followed by heating (˜1 min) with a heating gun (˜250° C.). Alternatively, a “Seebach staining solution” may be used (700 mL water, 10.5 g Cerium (IV) sulphate tetrahydrate, 15.0 g molybdato phosphoric acid, 17.5 mL sulphuric acid). Organic solutions were concentrated on Biichi R-114 rotatory evaporators at ˜25 torr at 25-30° C.

Commercial regents and solvents were used as received. All solvents used for extraction and chromatography were HPLC grade. Normal-phase Si gel Sep Paks™ were purchased from Waters, Inc. Thin-layer chromatography plates were Kieselgel 60F₂₅₄. All synthetic reagents were purchased from Sigma Aldrich or Fisher Scientific Canada.

Proton nuclear magnetic resonance (¹H NMR) spectra were recorded at 25° C. using a Bruker 400 with inverse probe and Bruker 400 spectrometers, are reported in parts per million on the δ scale, and are referenced from the residual protium in the NMR solvent (DMSO-d₆: δ 6 2.50 (DMSO-d₅)). Data is reported as follows: chemical shift [multiplicity (s=singlet, d=doublet, dd=doublet of doublets, m=multiplet, q=quintuplet, t=triplet), coupling constant(s) in Hertz, integration]. Carbon-13 nuclear magnetic resonance (¹³C NMR) spectra were recorded with a Bruker 400 spectrometer, are reported in parts per million on the 6 scale, and are referenced from the carbon resonances of the solvent (DMSO-d₆: δ 39.51). Data is reported as follows: chemical shift. Fluorine nuclear magnetic resonance (¹⁹F NMR) spectra were recorded at 25° C. using a Bruker 300 spectrometer, are reported in parts per million on the 6 scale.

### Cell Lines, Androgen and Reporters

LNCaP cells were employed initially for all experiments because they are well-differentiated human prostate cancer cells in which ligand-independent activation of the AR by FSK has been characterized (Nazareth et al 1996 J. Biol. Chem. 271, 19900-19907; and Sadar 1999 J. Biol. Chem. 274, 7777-7783). LNCaP cells express endogenous AR and secrete prostate-specific antigen (PSA) (Horoszewicz et al 1983 Cancer Res. 43, 1809-1818). LNCaP cells can be grown either as monolayers in cell culture or as tumors in the well-characterized xenograft model that progresses to androgen independence in castrated hosts (Sato et al 1996 J. Steroid Biochem. Mol. Biol. 58, 139-146; Gleave et al 1991 Cancer Res. 51, 3753-3761; Sato et al 1997 Cancer Res. 57, 1584-1589; and Sadar et al 2002 Mol. Cancer. Ther. 1(8), 629-637). PC3 human prostate cancer cells do not express functional AR (Kaighn et al 1978 Natl. Cancer Inst. Monogr. 49, 17-21) and were used to test specificity of compound for the AR. Small molecules that specifically target the AR-NTD should have no effect on PC3 cells. This means that they should not alter the proliferation of PC3 cells if they specifically block the AR to mediate their inhibitory effects. R1881 was employed since it is stable and avoids problems associated with the labile physiological ligand dihydrotestosterone (DHT). Reporter specificity may be determined using several alternative reporter gene constructs. Some well characterized ARE-driven reporter gene constructs that have been used extensively are the PSA (6.1 kb) enhance/promoter which contains several AREs and is highly inducible by androgens as well as by FSK (Ueda et al 2002 A J. Biol. Chem. 277, 7076-7085) and the ARR3-thymidine kinase (tk)-luciferase, which is an artificial reporter construct that contains three tandem repeats of the rat probasin ARE1 and ARE2 regions upstream of a luciferase reporter (Snoek et al 1996 J. Steroid Biochem. Mol. Biol. 59, 243-250). CMV-luc (no AREs and is constitutively active) was employed to determine that a compound does not have a general inhibitory effect on transcription.

### Example 1

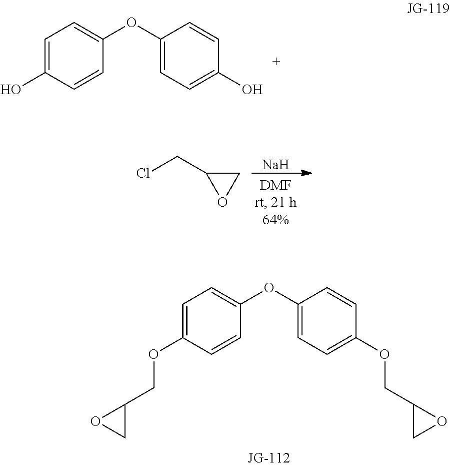

(JG-112). A round-bottomed flask was charged sequentially with NaH (148 mg, 3.71 mmol, 3 equiv), anhydrous dimethyl formamide (4 mL), and 4,4′-dihydroxydiphenyl ether (250 mg, 1.23 mmol, 1 equiv) and the contents were stirred under an atmosphere of argon for 10 min. Racemic epichlorohydrin (290 μL, 3.71 mmol, 3 equiv) was added via syringe and the mixture was allowed to react at room temperature for 21 h. Then, the solution was quenched with deionized water (˜2 mL) and the mixture was extracted with ethyl acetate (3×4 mL). The organic layer was washed with deionized water (2 mL), was dried over anhydrous magnesium sulfate, was filtered, and was concentrated under reduced pressure. The resulting residue was purified by flash column chromatography on silica gel (eluent: dichloromethane) to provide JG-112 (247 mg, 64%) as a white solid.

¹H NMR (400 MHz, DMSO-d₆): δ 6.94 (d, J=8.8, 4H), 6.89 (d, J=9.2, 4H), 4.27 (dd, J=11.2, 2.4, 2H), 3.79 (dd, J=11.2, 6.4, 2H), 3.30 (s, 2H), 2.82 (dd, J=4.8, 2H), 2.69 (m, 2H).

¹³C NMR (100 MHz, DMSO-d₆): δ 154.7, 151.8, 120.0, 116.3, 70.0, 50.3, 44.4.

HRMS (ESI) (m/z): calc'd for C₁₈H₁₈O₅Na [M+Na]⁺: 337.1052, found: 337.1099.

TLC (5% methanol in dichloromethane), Rt. 0.75 (UV, p-anisaldehyde).

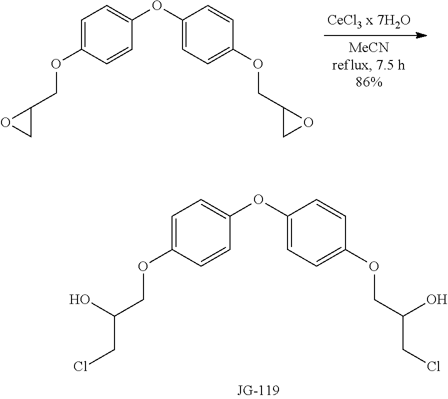 [SMILES: c1cc(Oc2ccc(OCC3CO3)cc2)ccc1OCC1CO1 ; OC(CCl)COc1ccc(Oc2ccc(OCC(O)CCl)cc2)cc1]

(JG-119). To a solution of racemic 4,4′-dihydroxydiphenyl ether diglycidyl ether JG-112 (100 mg, 0.318 mmol, 1 equiv) in acetonitrile (3.0 mL) was added CeCl₃.7H₂O (355 mg, 0.954 mmol, 3 equiv) and the mixture was refluxed for 7.5 h. The resulting white paste was filtered with dichloromethane and the clear suspension was concentrated under reduced pressure. The resulting residue was purified by flash column chromatography on silica gel (eluent: dichloromethane and 10% methanol in dichloromethane) to provide JG-119 (106 mg, 86%) as a white foam.

¹H NMR (400 MHz, DMSO-d₆): δ 6.90 (m, 8H), 5.52 (d, J=5.2, 2H), 4.02 (m, 2H), 3.94 (d, J=5.2, 4H), 3.74 (dd, J=11.2, 4.4, 2H), 3.65 (dd, J=11.2, 5.6, 2H).

¹³C NMR (100 MHz, DMSO-d₆): δ 154.8, 151.7, 120.0, 116.3, 70.1, 69.3, 47.3.

HRMS (ESI) (m/z): na

TLC (5% methanol in dichloromethane), Rt. 0.47 (UV, p-anisaldehyde).

### Example 2

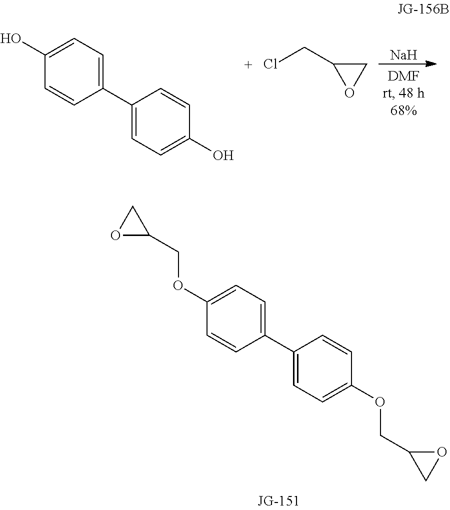

(JG-151). A round-bottomed flask was charged sequentially with NaH (322 mg, 8.05 mmol, 3 equiv), anhydrous dimethyl formamide (5 mL), and 4,4′-dihydroxybiphenyl (500 mg, 2.68 mmol, 1 equiv) and the contents were stirred under an atmosphere of argon for 10 min. Racemic epichlorohydrin (631 μL, 8.05 mmol, 3 equiv) was added via syringe and the mixture was allowed to react at room temperature for 48 h. Then, the solution was quenched with deionized water (˜2 mL) and the mixture was extracted with ethyl acetate (3×5 mL). The organic layer was washed with deionized water (3 mL), was dried over anhydrous magnesium sulfate, was filtered, and was concentrated under reduced pressure. The resulting residue was purified by washes (eluent: dichloromethane, ethyl acetate) to provide JG-151 (543 mg, 68%) as a pale solid.

¹H NMR (400 MHz, DMSO-d₆): δ 7.53 (d, J=8.4, 4H), 7.00 (d, J=8.4, 4H), 4.34 (dd, J=11.2, 2.4, 2H), 3.85 (dd, J=11.6, 6.8,

¹³C NMR (100 MHz, DMSO-d₆): δ 158.1, 133.1, 127.9, 115.5, 69.6, 50.3, 44.4.

HRMS (ESI) (m/z): calc'd for C₁₈H₁₈O₄Na [M+Na]⁺: 321.1103, found: 321.1106.

TLC (5% methanol in dichloromethane), Rf: 0.85 (UV, p-anisaldehyde).

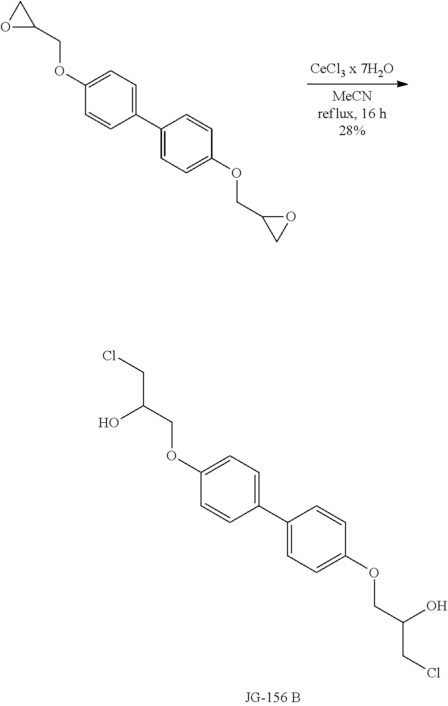 [SMILES: c1cc(-c2ccc(OCC3CO3)cc2)ccc1OCC1CO1 ; OC(CCl)COc1ccc(-c2ccc(OCC(O)CCl)cc2)cc1]

(JG-156 B). To a solution of racemic 4,4′-dihydroxybiphenyl diglycidyl ether JG-151 (100 mg, 0.33 mmol, 1 equiv) in acetonitrile (3.0 mL) was added CeCl₃.7H₂O (250 mg, 0.67 mmol, 2 equiv) and the mixture was refluxed for 16 h. The resulting white paste was filtered with dichloromethane and the clear suspension was concentrated under reduced pressure. The resulting residue was purified by flash column chromatography on silica gel (eluent: dichloromethane) to provide JG-156 B (35 mg, 28%) as a white solid.

¹H NMR (400 MHz, DMSO-d₆): δ 7.52 (d, J=8.4, 4H), 7.00 (d, J=8.8, 4H), 5.56 (d, J=5.2, 2H), 4.00 (m, 6H), 3.75 (dd, J=11.2, 4.4, 2H), 3.67 (dd, J=11.2, 5.2, 2H).

¹³C NMR (100 MHz, DMSO-d₆): δ 158.1, 133.1, 127.9, 115.5, 69.7, 69.2, 47.3.

HRMS (ESI) (m/z): na

TLC (5% methanol in dichloromethane), Rf. 0.58 (UV, p-anisaldehyde).

### Example 3

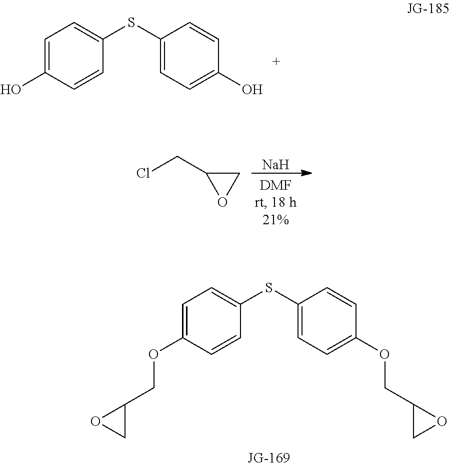

(JG-169). A round-bottomed flask was charged sequentially with NaH (275 mg, 6.87 mmol, 3 equiv), anhydrous dimethyl formamide (5 mL), and 4,4′-thiodiphenol (500 mg, 2.29 mmol, 1 equiv) and the contents were stirred under an atmosphere of argon for 10 min. Racemic epichlorohydrin (540 tit, 6.87 mmol, 3 equiv) was added via syringe and the mixture was allowed to react at room temperature for 18 h. Then, the solution was quenched with deionized water (˜2 mL) and the mixture was extracted with ethyl acetate (3×4 mL). The organic layer was washed with deionized water (2 mL), was dried over anhydrous magnesium sulfate, was filtered, and was concentrated under reduced pressure. The resulting residue was purified by flash column chromatography on silica gel (eluent: dichloromethane) to provide JG-169 (159 mg, 21%) as a white foam.

¹H NMR (400 MHz, DMSO-d₆): δ 7.24 (d, J=8.8, 4H), 6.94 (d, J=8.8, 4H), 4.30 (dd, J=11.2, 2.4, 2H), 3.80 (dd, J=11.2, 6.4, 2H), 3.30 (m, 2H), 2.83 (t, J=4.8, 2H), 2.68 (dd, J=4.8, 2.4, 2H).

¹³C NMR (100 MHz, DMSO-d₆): δ 158.4, 133.2, 127.3, 116.3, 69.7, 50.2, 44.3.

HRMS (ESI) (m/z): na

TLC (5% methanol in dichloromethane), Rf. 0.80 (UV, p-anisaldehyde).

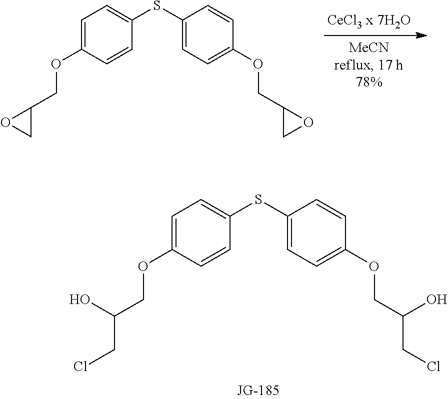 [SMILES: c1cc(Sc2ccc(OCC3CO3)cc2)ccc1OCC1CO1 ; OC(CCl)COc1ccc(Sc2ccc(OCC(O)CCl)cc2)cc1]

(JG-185). To a solution of racemic 4,4′-thiodiphenol diglycidyl ether JG-169 (18 mg, 0.05 mmol, 1 equiv) in acetonitrile (0.5 mL) was added CeCl₃.7H₂O (51 mg, 0.13 mmol, 2.5 equiv) and the mixture was refluxed for 17 h. The resulting white paste was filtered with dichloromethane and the clear suspension was concentrated under reduced pressure.

The resulting residue was purified by flash column chromatography on silica gel (eluent: dichloromethane and 10% methanol in dichloromethane) to provide JG-185 (17 mg, 78%) as a white foam.

¹H NMR (400 MHz, DMSO-d₆): δ 7.25 (d, J=8.4, 4H), 6.94 (d, J=8.8, 4H), 5.54 (d, J=5.2, 2H), 4.00 (m, 2H), 3.95 (m, 4H), 3.73 (dd, J=10.8, 4.4, 2H), 3.65 (dd, J=10.8, 5.2, 2H).

¹³C NMR (100 MHz, DMSO-d₆): δ 158.6, 133.2, 127.3, 116.3, 69.8, 69.2, 47.2.

HRMS (ESI) (m/z): calc'd for C₁₈H₂₀O₄NaSCl₂ [M+Na]⁺: 425.0357, found: 425.0345.

TLC (5% methanol in dichloromethane), Rt. 0.52 (UV, p-anisaldehyde).

### Example 4

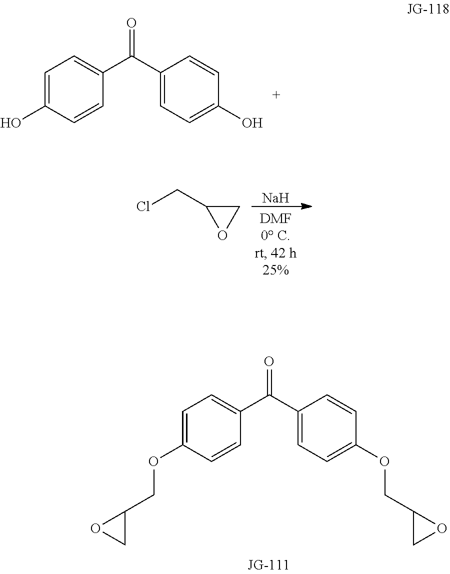

(JG-111). A round-bottomed flask was charged sequentially with NaH (205 mg, 5.13 mmol, 2.2 equiv), anhydrous dimethyl formamide (5 mL), and 4,4′-dihydroxybenzophenone (500 mg, 2.33 mmol, 1 equiv) at 0° C., and the contents were stirred under an atmosphere of argon for 10 min. Racemic epichlorohydrin (890 μL, 9.57 mmol, 4 equiv) was added via syringe and the mixture was allowed to react at room temperature for 44 h. Then, the solution was quenched with deionized water (˜2 mL) and the mixture was extracted with ethyl acetate (3×4 mL). The organic layer was washed with deionized water (2 mL), was dried over anhydrous magnesium sulfate, was filtered, and was concentrated under reduced pressure. The resulting residue was purified by flash column chromatography on silica gel (eluent: dichloromethane) to provide JG-111 (186 mg, 25%) as a white solid.

¹H NMR (400 MHz, DMSO-d₆): δ 7.70 (d, J=8.8, 4H), 7.10 (d, J=8.4, 4H), 4.44 (dd, J=11.6, 2.4, 2H), 3.94 (dd, J=11.2, 6.4,

¹³C NMR (100 MHz, DMSO-d₆): δ 193.7, 162.1, 132.4, 130.9, 114.9, 69.9, 50.2, 44.4.

HRMS (ESI) (m/z): calc'd for C₁₉H₁₉O₅ [M+H]⁺: 327.1232, found: 327.1234.

TLC (5% methanol in dichloromethane), R1: 0.55 (UV, p-anisaldehyde).

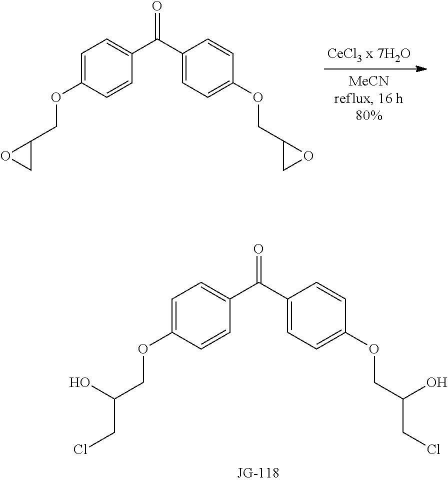 [SMILES: O=C(c1ccc(OCC2CO2)cc1)c1ccc(OCC2CO2)cc1 ; O=C(c1ccc(OCC(O)CCl)cc1)c1ccc(OCC(O)CCl)cc1]

(JG-118). To a solution of racemic 4,4′-dihydroxybenzophenone diglycidyl ether JG-111 (61 mg, 0.18 mmol, 1 equiv) in acetonitrile (2.0 mL) was added CeCl₃.7H₂O (171 mg, 0.46 mmol, 2.5 equiv) and the mixture was refluxed for 16 h. The resulting white paste was filtered with dichloromethane and the clear suspension was concentrated under reduced pressure. The resulting residue was purified by flash column chromatography on silica gel (eluent: dichloromethane and 10% methanol in dichloromethane) to provide JG-118 (60 mg, 80%) as a colourless foam.

¹H NMR (400 MHz, DMSO-d₆): δ 7.70 (d, J=8.8, 4H), 7.10 (d, J=8.8, 4H), 5.61 (d, J=3.6, 2H), 4.07 (m, 6H), 3.72 (m, 4H).

¹³C NMR (100 MHz, DMSO-d₆): δ 193.7, 162.3, 132.4, 130.9, 114.9, 69.9, 69.1, 47.1.

HRMS (ESI) (m/z): na

TLC (5% methanol in dichloromethane), Rf. 0.41 (UV, p-anisaldehyde).

### Example 5

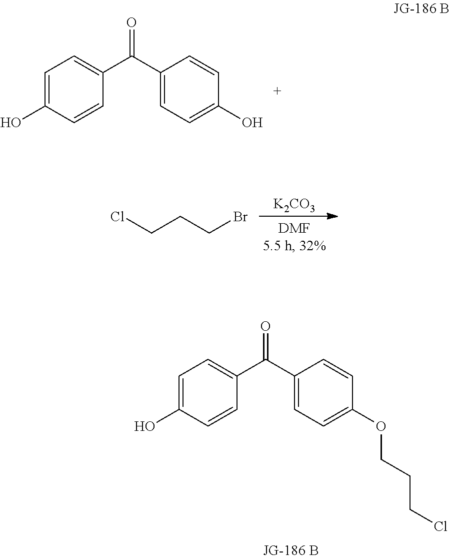

(JG-186 B). To a stirred solution of 4,4′-dihydroxybenzophenone (1000 mg, 4.66 mmol, 1 equiv) in anhydrous dimethyl formamide (5 mL) at rt was added K₂CO₃ (1.9 g, 14 mmol, 3 equiv) and the mixture was stirred for 10 min under argon atmosphere. 1-Bromo-3-chloropropane (1.4 mL, 14 mmol, 3 equiv) was added and the mixture was stirred for 5.5 h at rt. Deionized water (2 mL) was added and the mixture was extracted with ethyl acetate (3×3 mL). The organic layer was washed with deionized water (3 mL), was dried over anhydrous magnesium sulfate, was filtered, and was concentrated under reduced pressure. The resulting residue was purified by flash column chromatography on silica gel (eluent: dichloromethane) to provide JG-186 B (430 mg, 32%) as a white solid.

¹H NMR (400 MHz, DMSO-d₆): δ 10.35 (s, 1H), 7.67 (d, J=8.8, 2H), 7.61 (d, J=8.8, 2H), 7.01 (d, J=8.4, 2H), 6.87 (d, J=8.8, 2H), 4.19 (t, J=6.0, 2H), 3.80 (t, J=6.4, 2H), 2.20 (q, 2H).

¹³C NMR (100 MHz, DMSO-d₆): δ 193.7, 162.1, 162.0, 132.8, 132.3, 131.1, 129.1, 115.7, 114.8, 65.2, 42.5, 32.2.

HRMS (ESI) (m/z): na

TLC (5% methanol in dichloromethane), Rt. 0.55 (UV, p-anisaldehyde).

### Example 6

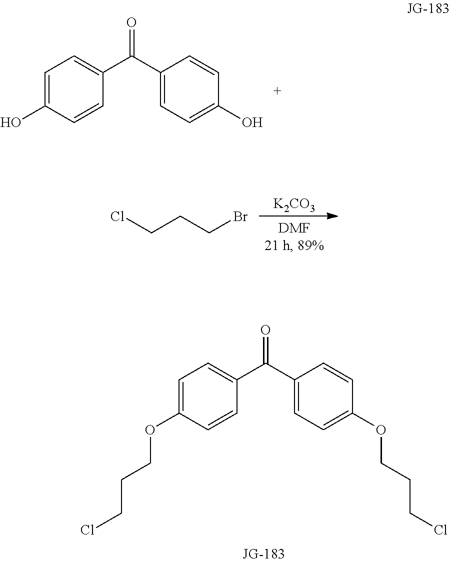

(JC-183). To a stirred solution of 4,4′-dihydroxybenzophenone (1000 mg, 4.66 mmol, 1 equiv) in anhydrous dimethyl formamide (5 mL) at rt was added K₂CO₃ (1.9 g, 14 mmol, 3 equiv) and the mixture was stirred for 20 min under argon atmosphere. 1-Bromo-3-chloropropane (1.4 mL, 14 mmol, 3 equiv) was added and the mixture was stirred for 21 h at rt. Deionized water (2 mL) was added and the mixture was extracted with ethyl acetate (3×3 mL). The organic layer was washed with deionized water (3 mL), was dried over anhydrous magnesium sulfate, was filtered, and was concentrated under reduced pressure. The resulting residue was purified by flash column chromatography on silica gel (eluent: 5% ethyl acetate in dichloromethane) to provide JG-183 (1.5 g, 89%) as a white solid.

¹H NMR (400 MHz, DMSO-d₆): δ 7.69 (d, J=8.8, 4H), 7.08 (d, J=8.4, 4H), 4.19 (t, J=6.0, 4H), 3.80 (t, J=6.4, 4H), 2.20 (q, 4H).

¹³C NMR (100 MHz, DMSO-d₆): δ 193.7, 162.3, 132.5, 130.8, 114.8, 65.3, 42.5, 32.2.

HRMS (ESI) (m/z): calc'd for C₁₉H₂₀O₃NaCl₂ [M+Na]⁺: 389.0687, found: 389.0692.

TLC (5% methanol in dichloromethane), Rt. 0.82 (UV, p-anisaldehyde).

### Example 7

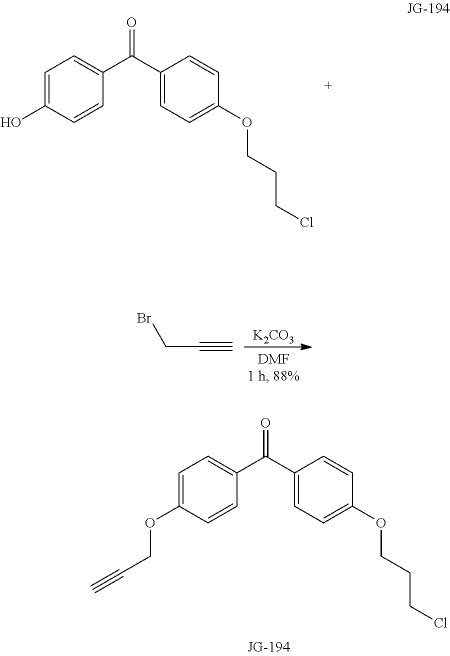

(JG-194). To a stirred solution of 4,4′-dihydroxybenzophenone derivative JG-186 (30 mg, 0.10 mmol, 1 equiv) in anhydrous dimethyl formamide (0.5 mL) at rt was added K₂CO₃ (29 mg, 0.20 mmol, 2 equiv) and the mixture was stirred for 20 min under argon atmosphere. Propargyl bromide (19 μL, 0.2 mmol, 2 equiv) was added and the mixture was stirred for 1 h at rt. Deionized water (0.2 mL) was added and the mixture was extracted with ethyl acetate (3 xl mL) The organic layer was washed with deionized water (1 mL), was dried over anhydrous magnesium sulfate, was filtered, and was concentrated under reduced pressure. The resulting residue was purified by flash column chromatography on silica gel (eluent: 5% ethyl acetate in dichloromethane) to provide JG-194 (30 mg, 88%) as a white solid.

¹H NMR (400 MHz, DMSO-d₆): δ 7.71 (d, J=8.4, 4H), 7.11 (t, J=8.4, 4H), 4.91 (d, J=1.6, 2H), 4.19 (t, J=6.0, 2H), 3.81 (t, J=6.4, 2H), 3.64 (s, 1H), 2.20 (q, 2H).

¹³C NMR (100 MHz, DMSO-d₆): δ 193.8, 162.3, 161.0, 132.5, 132.3, 131.3, 130.7, 115.2, 114.9, 79.3, 79.3, 65.3, 56.3, 42.5, 32.2.

HRMS (ESI) (m/z): na

TLC (5% methanol in dichloromethane), Rf. 0.92 (UV, p-anisaldehyde).

### Example 8

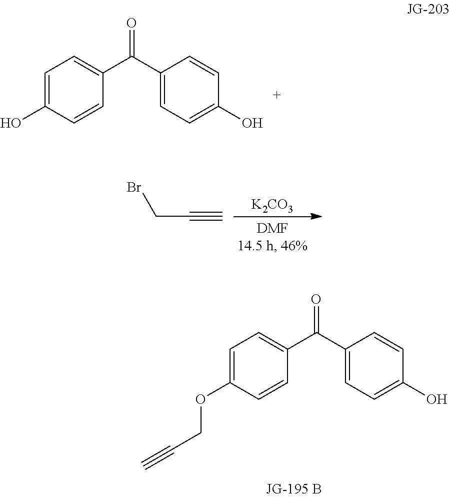

(JG-195 B). To a stirred solution of 4,4′-dihydroxybenzophenone (250 mg, 1.16 mmol, 1 equiv) in anhydrous dimethyl formamide (2 mL) at rt was added K₂CO₃ (242 mg, 1.75 mmol, 1.5 equiv) and the mixture was stirred for 20 min under argon atmosphere. Propargyl bromide (156 μL, 1.75 mmol, 1.5 equiv) was added and the mixture was stirred for 14.5 h at rt. Deionized water (0.5 mL) was added and the mixture was extracted with ethyl acetate (3×2 mL). The organic layer was washed with deionized water (2 mL), was dried over anhydrous magnesium sulfate, was filtered, and was concentrated under reduced pressure. The resulting residue was purified by flash column chromatography on silica gel (eluent: 5% ethyl acetate in dichloromethane) to provide JC-195 B (135 mg, 46%) as a white solid.

¹H NMR (400 MHz, DMSO-d₆): 10.32 (s, 1H), 7.68 (d, J=8.8, 2H), 7.62 (d, J=8.8, 2H), 7.11 (d, J=8.8, 2H), 6.88 (d, J=8.4, 2H), 4.91 (d, J=2.4, 2H), 3.63 (t, J=2.0, 1H).

¹³C NMR (100 MHz, DMSO-d₆): δ 193.7, 162.2, 160.8, 132.8, 132.1, 131.6, 129.0, 115.7, 115.1, 79.4, 79.3, 56.3.

HRMS (ESI) (m/z): na

TLC (5% methanol in dichloromethane), Rt. 0.48 (UV, p-anisaldehyde).

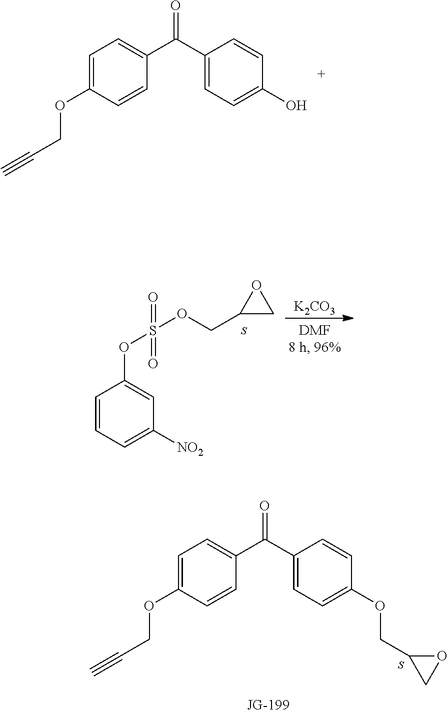

(JG-199). A round-bottomed flask was charged sequentially with K₂CO₃ (44 mg, 0.32 mmol, 2 equiv), anhydrous dimethyl formamide (1 mL), and 4,4′-dihydroxybenzophenone derivative JG-195 B (40 mg, 0.15 mmol, 1 equiv) and the contents were stirred under an atmosphere of argon for 20 min. (+)—S-glycidyl nosylate (83 mg, 0.32 mmol, 2 equiv) was added via syringe dissolved in anhydrous dimethyl formamide (0.3 mL) and the mixture was allowed to react at room temperature for 8 h. Then, the solution was quenched with deionized water (˜0.5 mL) and the mixture was extracted with ethyl acetate (3×2 mL). The organic layer was washed with deionized water (2 mL), was dried over anhydrous magnesium sulfate, was filtered, and was concentrated under reduced pressure. The resulting residue was purified by flash column chromatography on silica gel (eluent: 5% ethyl acetate in dichloromethane) to provide JG-199 (47 mg, 96%) as a white solid.

¹H NMR (400 MHz, DMSO-d₆): δ 7.70 (dd, J=8.8, 2.0, 4H), 7.11 (t, J=8.0, 4H), 4.91 (d, J=2.4, 2H), 4.44 (dd, J=11.2, 2.4, 1H), 3.94 (dd, J=11.6, 6.4, 1H), 3.63 (t, J=2.0, 1H), 3.37 (m, 1H), 2.86 (t, J=4.8, 1H), 2.73 (dd, J=5.2, 2.8, 1H).

¹³C NMR (100 MHz, DMSO-d₆): δ 193.7, 162.2, 161.0, 132.5, 132.3, 131.3, 130.8, 116.2, 114.9, 79.4, 79.3, 69.9, 56.3, 55.5, 50.1, 44.4.

HRMS (ESI) (m/z): na

TLC (5% methanol in dichloromethane), Rt. 0.73 (UV, p-anisaldehyde).

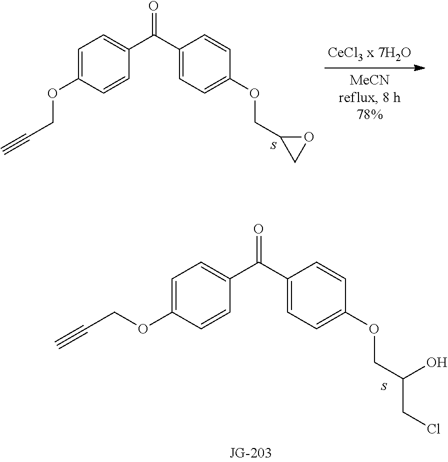

(JG-203). To a solution of S-4,4′-dihydroxybenzophenone glycidyl ether derivative JG-199 (18 mg, 0.06 mmol, 1 equiv) in acetonitrile (0.5 mL) was added CeCl₃′7H₂O (60 mg, 0.16 mmol, 2.6 equiv) and the mixture was refluxed for 8 h. The resulting white paste was filtered with dichloromethane and the clear suspension was concentrated under reduced pressure. The resulting residue was purified by flash column chromatography on silica gel (eluent: dichloromethane and 10% methanol in dichloromethane) to provide JG-203 (17 mg, 78%) as a colourless oil.

¹H NMR (400 MHz, DMSO-d₆): δ 7.71 (dd, J=8.8, 2.0, 4H), 7.11 (dd, J=10.4, 8.8, 4H), 4.91 (d, J=2.4, 2H), 4.08 (m, 3H), 3.76 (m, 1H), 3.68 (m, 1H), 3.63 (t, J=2.0, 1H).

¹³C NMR (100 MHz, DMSO-d₆): δ 193.7, 162.4, 161.0, 132.5, 132.3, 131.3, 130.7, 115.2, 114.9, 79.4, 79.3, 69.9, 69.1, 56.3, 47.2.

HRMS (ESI) (m/z): na

TLC (5% methanol in dichloromethane), Rf. 0.48 (UV, p-anisaldehyde).

### Example 9

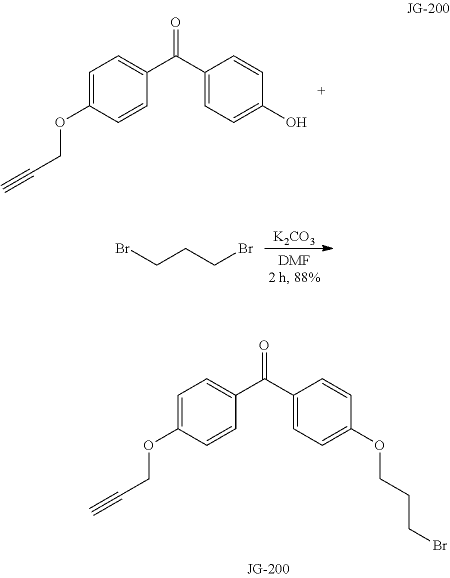

(JG-200). To a stirred solution of 4,4′-dihydroxybenzophenone derivative JG-195 B (10 mg, 0.04 mmol, 1 equiv) in anhydrous dimethyl formamide (0.4 mL) at rt was added K₂CO₃ (11 mg, 0.08 mmol, 2 equiv) and the mixture was stirred for 20 min under argon atmosphere. 1,3-Dibromopropane (40 μL, 0.39 mmol, 10 equiv) was added and the mixture was stirred for 2 h at rt. Deionized water (0.2 mL) was added and the mixture was extracted with ethyl acetate (3 xl mL). The organic layer was washed with deionized water (1 mL), was dried over anhydrous magnesium sulfate, was filtered, and was concentrated under reduced pressure. The resulting residue was purified by flash column chromatography on silica gel (eluent: 5% ethyl acetate in dichloromethane) to provide JG-200 (13 mg, 88%) as a colourless oil.

¹H NMR (400 MHz, DMSO-d₆): δ 7.71 (dd, J=8.8, 1.6, 4H), 7.11 (dd, J=10.8, 8.8, 4H), 4.91 (d, J=2.4, 2H), 4.19 (t, J=6.0, 2H), 3.68 (t, J=6.4, 2H), 3.63 (t, J=2.0, 1H), 2.28 (q, 2H).

¹³C NMR (100 MHz, DMSO-d₆): δ 193.7, 162.3, 161.0, 132.5, 132.3, 131.3, 130.7, 115.2, 114.9, 79.4, 79.3, 66.3, 56.3, 32.3, 31.7.

HRMS (ESI) (m/z): na

TLC (5% methanol in dichloromethane), Rf. 0.79 (UV, p-anisaldehyde).

### Example 10

LNCaP cells were transiently cotransfected with PSA (6.1 kb)-luciferase (0.25 μg/well) in 24-well plates for 24 h prior to pre-treatment with compounds for 1 hour before the addition of synthetic androgen, R1881 (1 nM) to induce PSA production or vehicle. The total amount of plasmid DNA transfected was normalized to 0.75 μg/well by the addition of the empty vector. After 48 h of incubation with R1881, the cells were harvested, and relative luciferase activity was determined. Test compounds were added to the cells at various concentrations and activity for each treatment was normalized to the predicted maximal activity induction (in the absence of test compounds, vehicle only). Plotting of sigmoidal curves (Boltzmann Function) and IC50 calculations were done using OriginPro 8.1 Software (Northampton, Mass., USA).

Furthermore, toxicity was assessed by both microscopic examination and reduction of protein levels. Solubility was assessed both macroscopically (cloudy media) and microscopically (formation of granules or crystals).

TABLE 3 shows the chemical structures for the compounds that showed activity using the above-described assays.

The following Table includes active compounds.

TABLE 3

| COMPOUND | EXPERIMENTAL DATA |
|---|---|
| 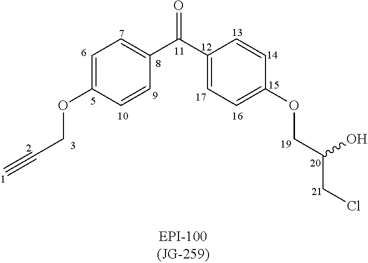 [SMILES: C#CCOc1ccc(C(=O)c2ccc(OCC(O)CCl)cc2)cc1] | Active |
| 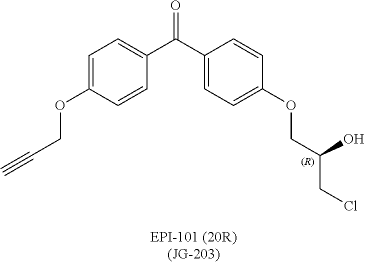 | Active |
| 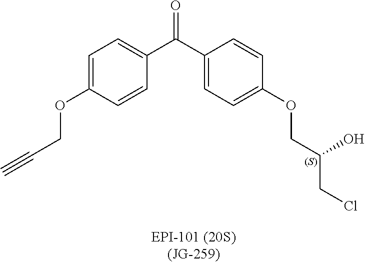 | Active |
| 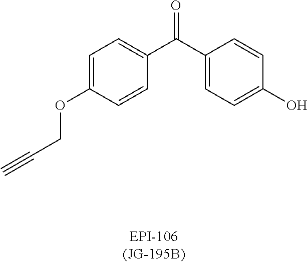 [SMILES: C#CCOc1ccc(C(=O)c2ccc(O)cc2)cc1] | Active |
| 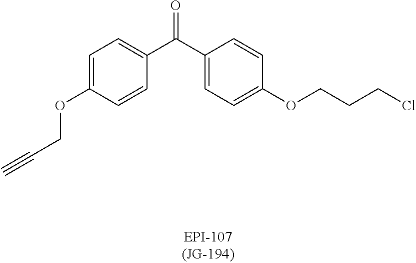 [SMILES: C#CCOc1ccc(C(=O)c2ccc(OCCCCl)cc2)cc1] | Active |
| 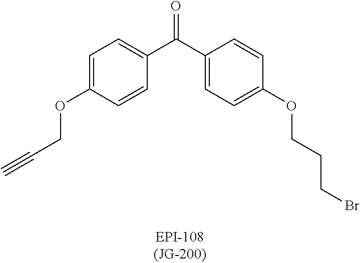 [SMILES: C#CCOc1ccc(C(=O)c2ccc(OCCCBr)cc2)cc1] | Active |
| 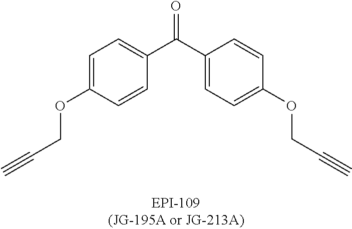 [SMILES: C#CCOc1ccc(C(=O)c2ccc(OCC#C)cc2)cc1] | Active |
| 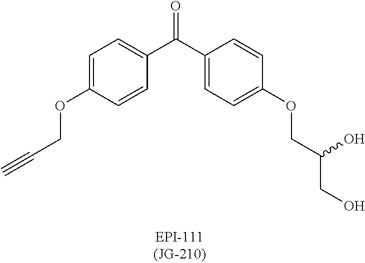 [SMILES: C#CCOc1ccc(C(=O)c2ccc(OCC(O)CO)cc2)cc1] | Active |
| 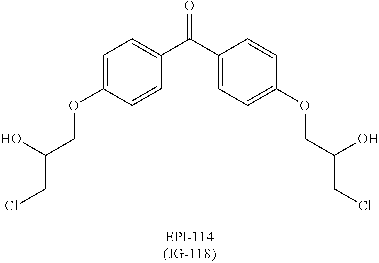 [SMILES: O=C(c1ccc(OCC(O)CCl)cc1)c1ccc(OCC(O)CCl)cc1] | Active |
| 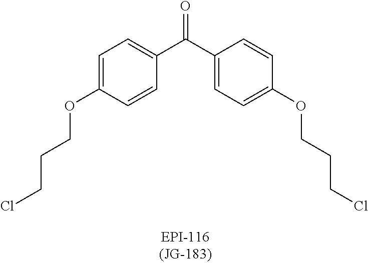 [SMILES: O=C(c1ccc(OCCCCl)cc1)c1ccc(OCCCCl)cc1] | Active |
| 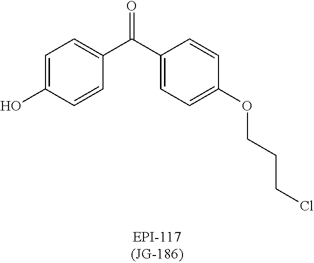 [SMILES: O=C(c1ccc(O)cc1)c1ccc(OCCCCl)cc1] | Active |
| 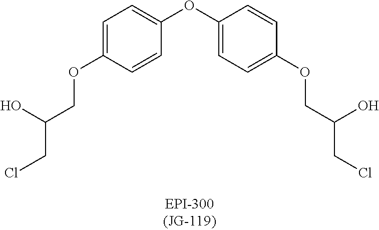 [SMILES: OC(CCl)COc1ccc(Oc2ccc(OCC(O)CCl)cc2)cc1] | Active |
| 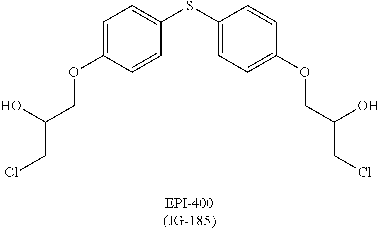 [SMILES: OC(CCl)COc1ccc(Sc2ccc(OCC(O)CCl)cc2)cc1] | Active |
| 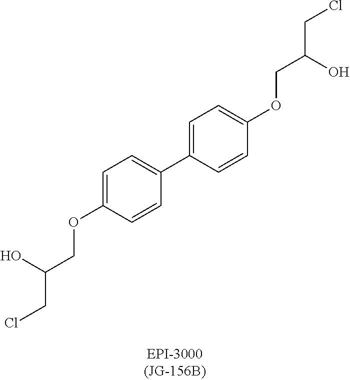 [SMILES: OC(CCl)COc1ccc(-c2ccc(OCC(O)CCl)cc2)cc1] | Active |
| 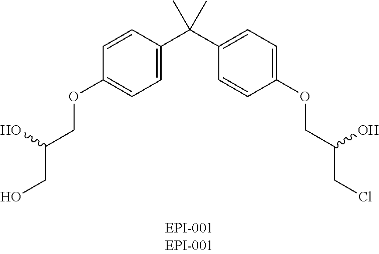 [SMILES: CC(C)(c1ccc(OCC(O)CO)cc1)c1ccc(OCC(O)CCl)cc1] | Active (positive control) |

### Example 11

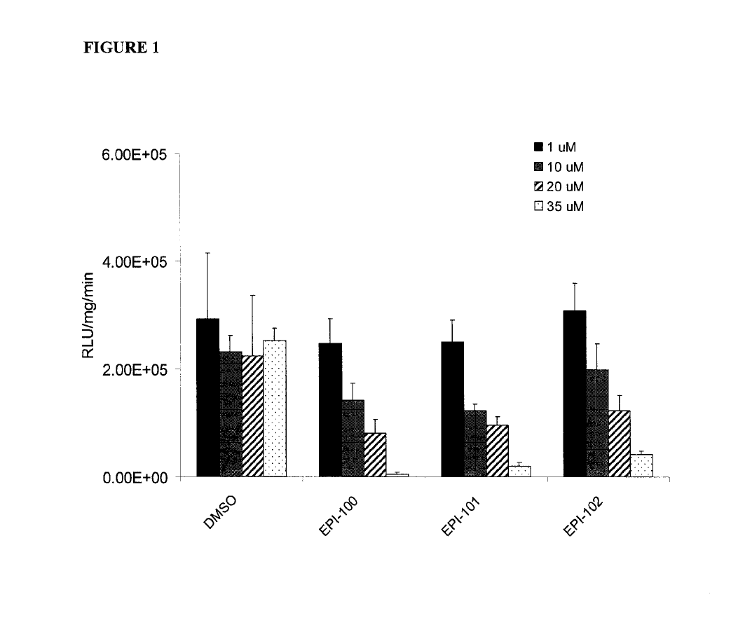

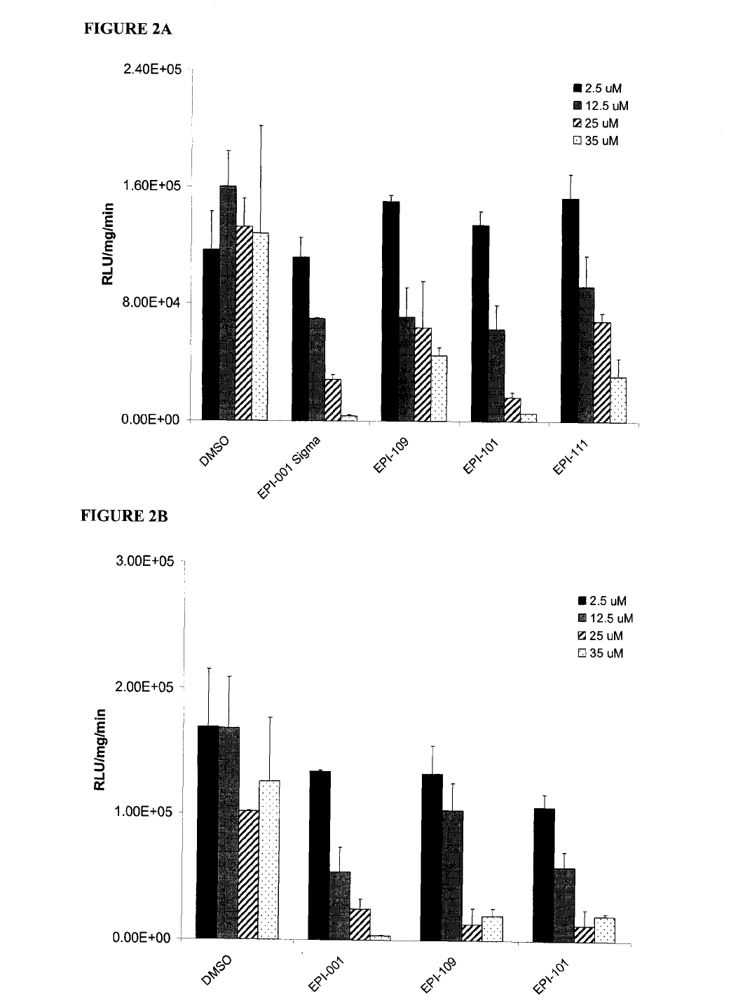

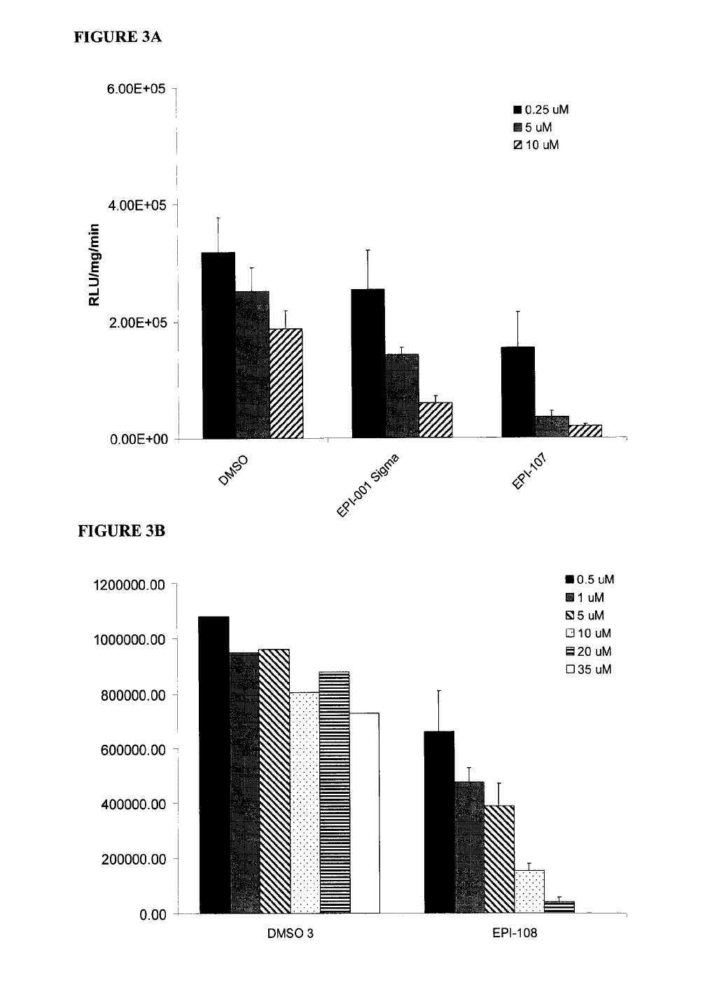

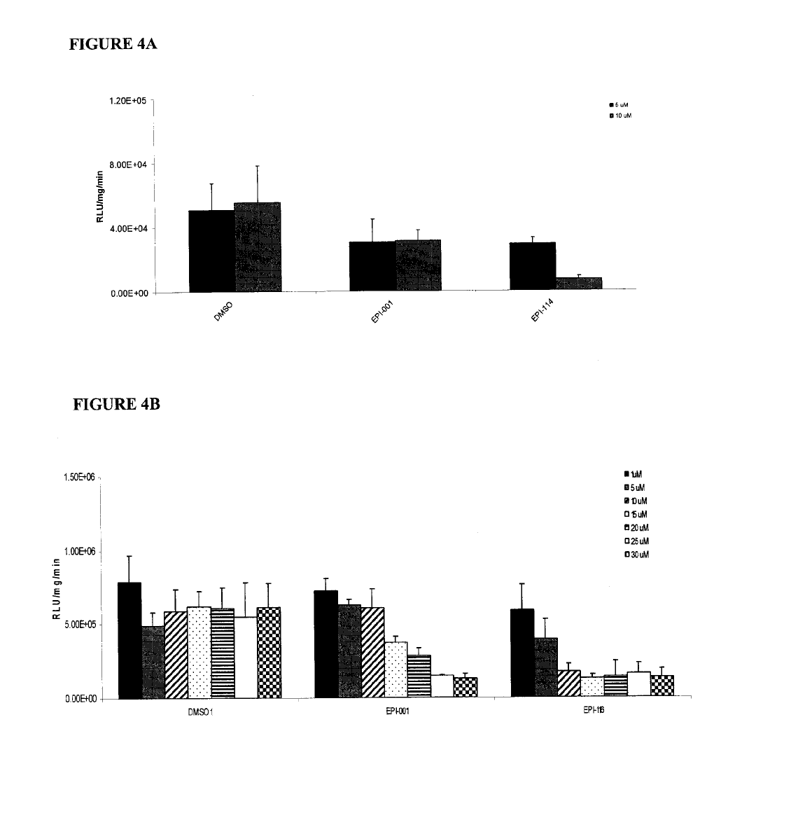

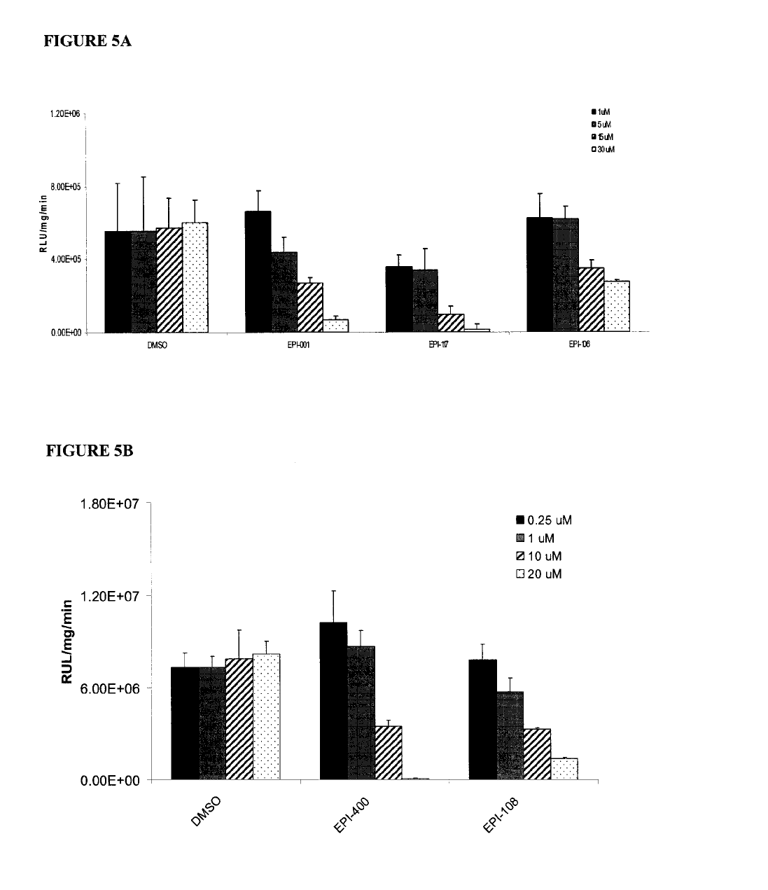

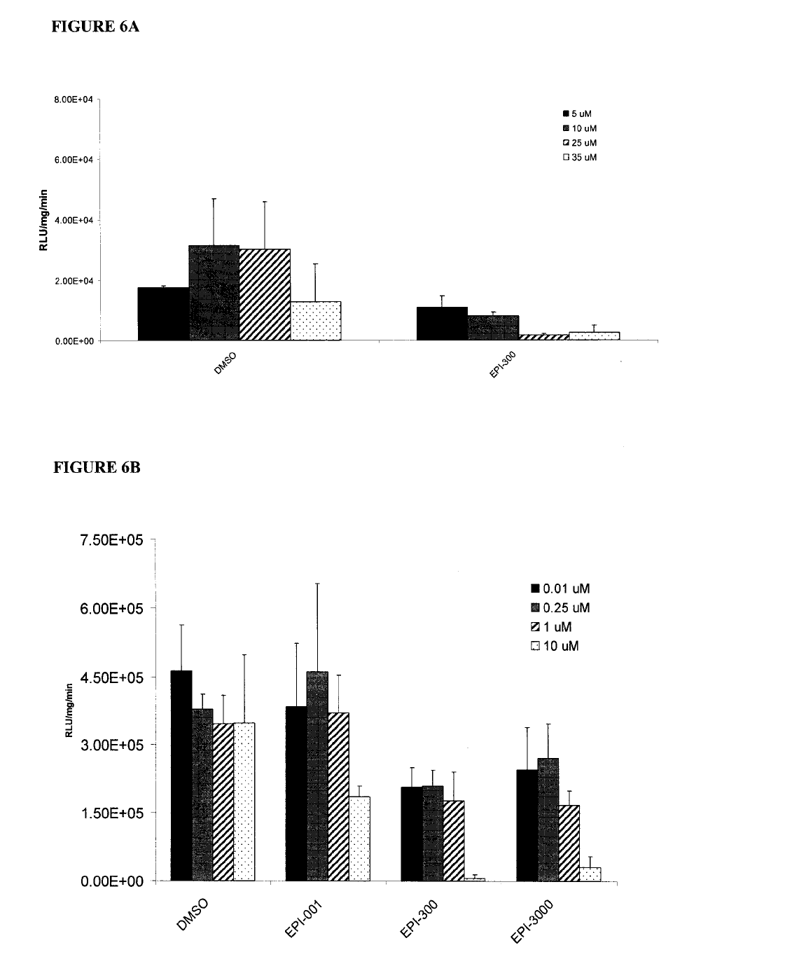

Using the LNCaP PSA (6.1 kb)-luciferase assay described above, various compounds described herein were tested against DMSO and EPI-001 controls. In FIG. 1, a comparison is made between EPI-100, EPI-101, EPI-102 and DMSO from 1 to 35 μM. FIG. 2A shows EPI-109, EPI-101 and EPI-111 as compared to DMSO and EPI-001 at 2.5 μM to 35 μM (2.5 μM, 12.5 μM, 25 μM, and 35 μM). FIG. 2B shows EPI-109 and EPI-101 as compared to DMSO and EPI-001 at 2.5 μM, 12.5 μM, 25 μM, and 35 μM. In FIG. 3A, EPI-107 is compared to DMSO and EPI-001 at 2.5 μM to 10 μM. In FIG. 3B, EPI-108 is compared to DMSO at 0.5 μM to 35 μM. In FIG. 4A, EPI-114 is compared to DMSO and EPI-001 at 5 μM and 10 μM. In FIG. 4B, EPI-116 is compared to DMSO and EPI-001 at 1 μM to 30 μM. FIG. 5A shows EPI-117 and EPI-106 in comparison to DMSO and EPI-001 at 1.0 μM to 30 μM. FIG. 5B shows EPI-400 and EPI-108 in comparison to DMSO at 0.25 μM to 20 μM. FIG. 6A, shows EPI-300 compared to DMSO at 5 μM to 35 μM. FIG. 6B, shows EPI-300 and EPI-3000 compared to DMSO and EPI-001 at 0.01 μM to 10 μM.

Each of EPI-100, EPI-101, EPI-102, EPI-106, EPI-107, EPI-108, EPI-109, EPI-111, EPI-114, EPI-116, EPI-117, EPI-300, EPI-400, and EPI-3000 showed androgen receptor inhibitory activity.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A compound having a structure of Formula I 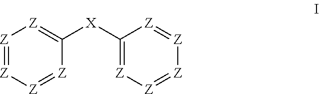
or a pharmaceutically acceptable salt thereof, wherein:
X is C═O, C(OH)₂, C(OR¹)₂, C(OH)(OR¹), C(OR¹)(OR²), O, S, SO, C═NOH, C═NR¹, CHNH₂, CHNHR¹, CHNHR², CHN(R¹)₂, CHN(R²)₂, or CHNR¹R²;
each R¹ is independently linear or branched, substituted or unsubstituted, saturated or unsaturated C₁-C₁₀ alkyl, and each R² is independently C₁-C₁₀ acyl, or two of the R¹ groups are joined to form a cyclic ketal, wherein the optional substituent may be selected from the group consisting of oxo, COOH, R³, OH, OR³, F, Cl, Br, I, NH₂, NHR³, N(R³)₂, CN, SH, SR³, SO₃H, SO₃R³, SO₂R³, OSO₃R³, OR⁶, CO₂R³, CONH₂, CONHR³, CONHR⁶, CON(R³)₂, NHR⁶, OPO₃H₃, CONR³R⁶, NR³R⁶, and NO₂;
each R³ is independently unsubstituted C₁-C₁₀ alkyl;
each R⁶ is independently C₁-C₁₀ acyl;
at least one Z of one aromatic ring is independently C-Q, at least one Z of the other aromatic ring is independently C-T, CF, CCl, CBr, CI, COH, CG¹, CNH₂, CNG¹₂, COSO₃H, COPO₃H₂, CSG¹, CSOG¹, or CSO₂G¹, and each remaining Z is independently C-T, N, CH, CF, CCl, CI, COH, CG¹, COG¹, CNH₂, CNHG¹, CNG¹₂, COSO₃H, COPO₃H₂, CSG¹, CSOG¹, or CSO₂G¹;
Q is 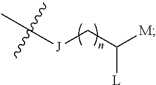
J is G¹, O, CH₂, CHG¹, CG¹₂, SO, or NR;
M is H, Cl, Br, CH₂Cl, CHCl₂, CCl₃, CH₂Br, CHBr₂, CBr₃, or C≡CH;
L is H or A-D;
A is O, S, NH, NG¹, N⁺H₂, or N⁺HG¹; 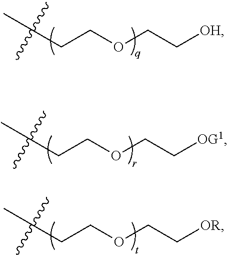
D is H, G¹, R,
or a moiety selected from TABLE 1;
each of q, r and t is independently 0, 1, 2, 3, 4, 5, 6 or 7;
n is 0, 1, 2, 3, 4, 5, 6, 7 or 8;
T is 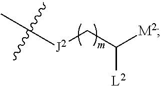
J² is G¹, O, CH₂, CHG¹, CG¹₂, S, NH, SO, SO₂, or NR;
M² is H, CH₃, Cl, Br, CH₂Cl, CHCl², CCl₃, CH₂Br, CHBr₂, CBr₃, CH₂OH, CH₂OJ″, G¹, CH₂OG¹, CH₂OR, CH₂OG¹OG¹′, G¹OG¹′, G¹′OG¹′OG¹″, CH₂SG¹, CH₂NH₂, CH₂NHG¹, CH₂NG¹₂, or C≡CH;
L² is H or A²-D²;
A² is O, S, SO, SO₂, NH, NG¹, N⁺H₂, or N⁺HG¹;
D² is H, G¹, R, 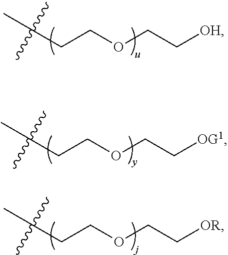
or a moiety selected from TABLE 1;
each of u, y and j is independently 0, 1, 2, 3, 4, 5, 6 or 7;
m is 0, 1, 2, 3, 4, 5, 6, 7 or 8;
each of J″ and J′″ is independently a moiety selected from TABLE 1;
each G¹ G¹′ and G¹′″ is independently linear or branched, substituted or unsubstituted, saturated or unsaturated C₁-C₁₀ alkyl, wherein the optional substituent may be selected from the group consisting of oxo, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, OR^{S}, CO₂R⁴, CONH₂, CONHR⁴, CONHR⁵, CON(R⁴)₂, NHR⁵, OPO₃H₃, CONR⁴R⁵, NR⁴R⁵, and NO₂;
each R⁴ is independently unsubstituted C₁-C₁₀ alkyl;
each R⁵ is independently C₁-C₁₀ acyl; and
R is C₁-C₁₀ acyl.

2. The compound or pharmaceutically acceptable salt of claim 1, wherein the at least one Z of the other aromatic ring is selected from: C-T; CF; CCl; CBr; CI; CG¹; CNH₂; and CNG¹₂.

3. The compound or pharmaceutically acceptable salt of claim 1, wherein the at least one Z of the other aromatic ring is selected from: C-T; and
CG¹.

4. The compound or pharmaceutically acceptable salt of claim 1, wherein each of the remaining Z is independently selected from: N; CG¹; CH;
CF; CCl; CBr; and CI.

5. The compound or pharmaceutically acceptable salt of claim 1, wherein each of the remaining Z is independently selected from: CG¹; CH; CCl; and CBr.

6. The compound or pharmaceutically acceptable salt of claim 1, wherein each of the remaining Z is independently selected from: CG¹; CH; and CBr.

7. The compound or pharmaceutically acceptable salt of claim 1, wherein CG¹ is CCH₃.

8. The compound or pharmaceutically acceptable salt of claim 7, wherein each remaining Z is independently selected from: CCH₃; CH; and CBr.

9. The compound or pharmaceutically acceptable salt of claim 1, wherein J is selected from: O; S; SO; and SO₂.

10. The compound or pharmaceutically acceptable salt of claim 1, wherein J is selected from: O; and S.

11. The compound or pharmaceutically acceptable salt of claim 1, wherein J is O.

12. The compound or pharmaceutically acceptable salt of claim 1, wherein M is selected from: H; Cl; Br; CH₂Cl; CHCl₂; CH₂Br; CHBr₂; CH₂OG¹; and C≡CH.

13. The compound or pharmaceutically acceptable salt of claim 1, wherein M is selected from: H; Cl; Br; CH₂Cl; CH₂Br; CH₂OG¹; and C≡CH.

14. The compound or pharmaceutically acceptable salt of claim 1, wherein M is selected from: Cl; Br; CH₂Cl; and CH₂Br.

15. The compound or pharmaceutically acceptable salt of claim 1, wherein M is selected from: CH₂Cl; CH₂Br; and C≡CH.

16. The compound or pharmaceutically acceptable salt of claim 1, wherein M is CH₂Cl.

17. The compound or pharmaceutically acceptable salt of claim 1, wherein L is H.

18. The compound or pharmaceutically acceptable salt of claim 1, wherein L is A-D.

19. The compound or pharmaceutically acceptable salt of claim 1, wherein A is selected from: O; S; and NH.

20. The compound or pharmaceutically acceptable salt of claim 1, wherein A is O.

21. The compound or pharmaceutically acceptable salt of claim 1, wherein D is selected from: H; 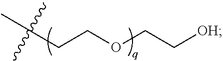
and a moiety selected from TABLE 1.

22. The compound or pharmaceutically acceptable salt of claim 1, wherein J² is selected from: O; S; SO; NH; and SO₂.

23. The compound or pharmaceutically acceptable salt of claim 1, wherein J² is selected from: O; and S.

24. The compound or pharmaceutically acceptable salt of claim 1, wherein J² is O.

25. The compound or pharmaceutically acceptable salt of claim 1, wherein M² is selected from: H; Cl; Br; CH₂Cl; CHCl₂; CH₂Br; CHBr₂; CH₂OH; CH₂OJ″; CH₂OG¹; and C≡CH.

26. The compound or pharmaceutically acceptable salt of claim 1, wherein M² is selected from: H; Cl; Br; CH₂Cl; CH₂Br; CH₂OG¹; and C≡CH.

27. The compound or pharmaceutically acceptable salt of claim 1, wherein M² is selected from: Cl; Br; CH₂OG¹; CH₂Cl; and CH₂Br.

28. The compound or pharmaceutically acceptable salt of claims 1, wherein M² is selected from: CH₂Cl; CH₂Br; and C≡CH.

29. The compound or pharmaceutically acceptable salt of claim 1, wherein M² is C≡CH.

30. The compound or pharmaceutically acceptable salt of claim 1, wherein L² is H.

31. The compound or pharmaceutically acceptable salt of claim 1, wherein L² is A²-D².

32. The compound or pharmaceutically acceptable salt of claim 1, wherein A² is selected from: O; S; and NH.

33. The compound or pharmaceutically acceptable salt of claim 1, wherein A² is O.

34. The compound or pharmaceutically acceptable salt of claim 1 wherein D² is selected from: H; 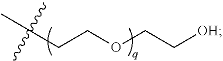
and a moiety selected from TABLE 1.

35. The compound or pharmaceutically acceptable salt of claim 1, wherein each G¹ G¹′ and G¹′″ is independently a linear or branched, substituted or unsubstituted, saturated or unsaturated C₁-C₁₀ alkyl, wherein the optional substituent may be selected from the group consisting of: oxo; Or; COOH; OH; F; Cl; Br; I; NH₂; CN; SH; SO₃H; CONH₂; OPO₃H₃; and NO₂.

36. The compound or pharmaceutically acceptable salt of claim 1, wherein each G¹ G¹′ and G¹′″ is independently a linear or branched, substituted or unsubstituted, saturated or unsaturated C₁-C₁₀ alkyl, wherein the optional substituent may be selected from the group consisting of: oxo; Or; COOH; OH; Cl; Br; NH₂; and NO₂.

37. The compound or pharmaceutically acceptable salt of claim 1, wherein X is selected from the group consisting of: C═O; O; S; and SO.

38. The compound or pharmaceutically acceptable salt thereof of claim 1, wherein the compound is selected from:

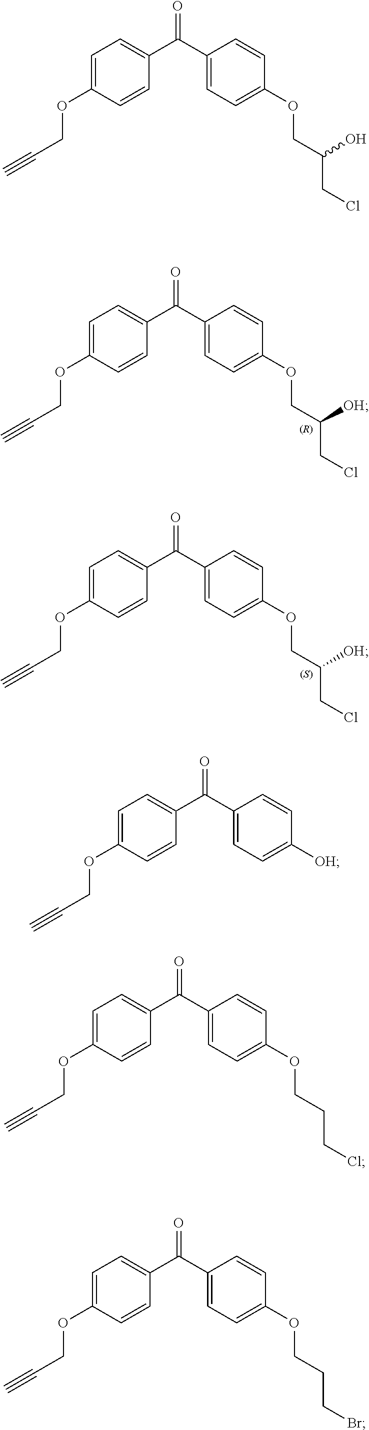

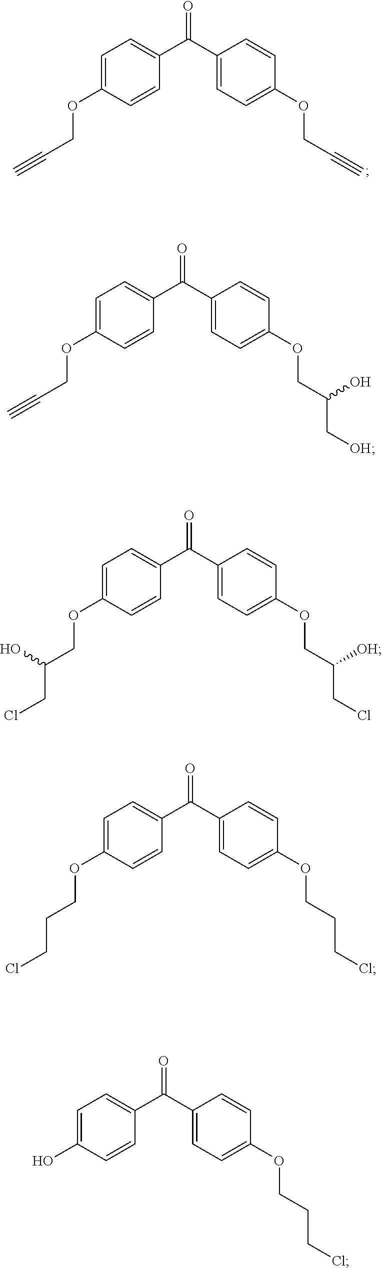

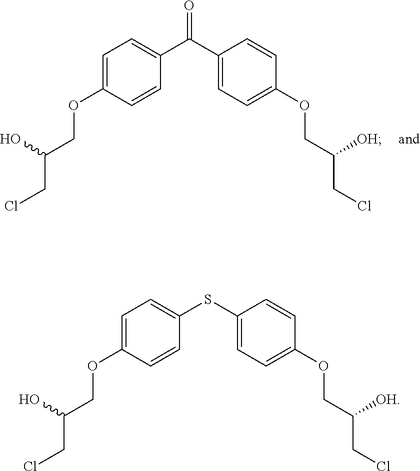 [SMILES: O=C(c1ccc(OCC(O)CCl)cc1)c1ccc(OC[C@H](O)CCl)cc1 ; "and" ; OC(CCl)COc1ccc(Sc2ccc(OC[C@H](O)CCl)cc2)cc1]

39. The compound or pharmaceutically acceptable of claim 1, wherein the compound is selected from TABLE 2.

40. The compound of claim 1, wherein the compound has the following Formula V: 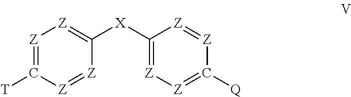
or a pharmaceutically acceptable salt thereof,
wherein:
Q is selected from 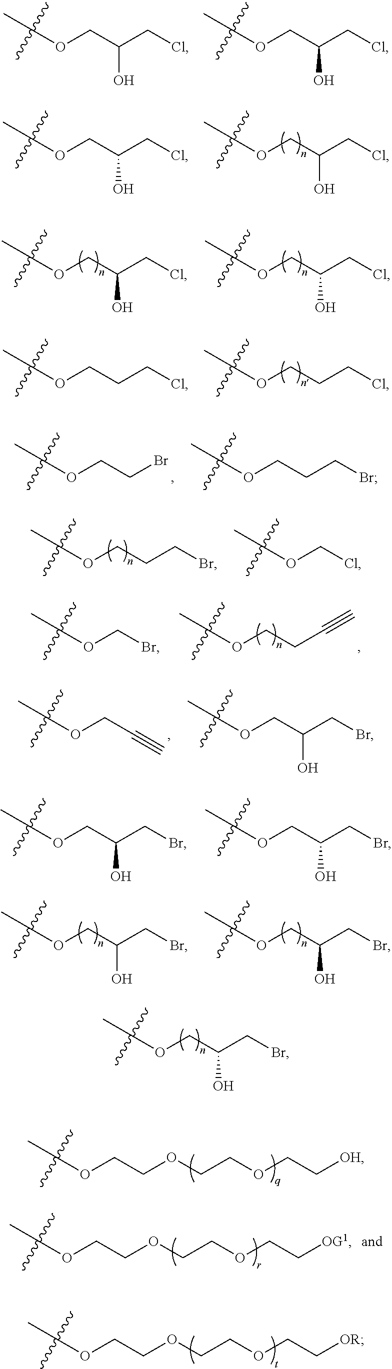
n′ is 1, 2, 3, 4, 5, 6, 7 or 8;
n is 0, 1, 2, 3,4,5,6,7 or 8;
each of q, r and t is independently 0, 1, 2, 3, 4, 5, 6 or 7;
T is selected from 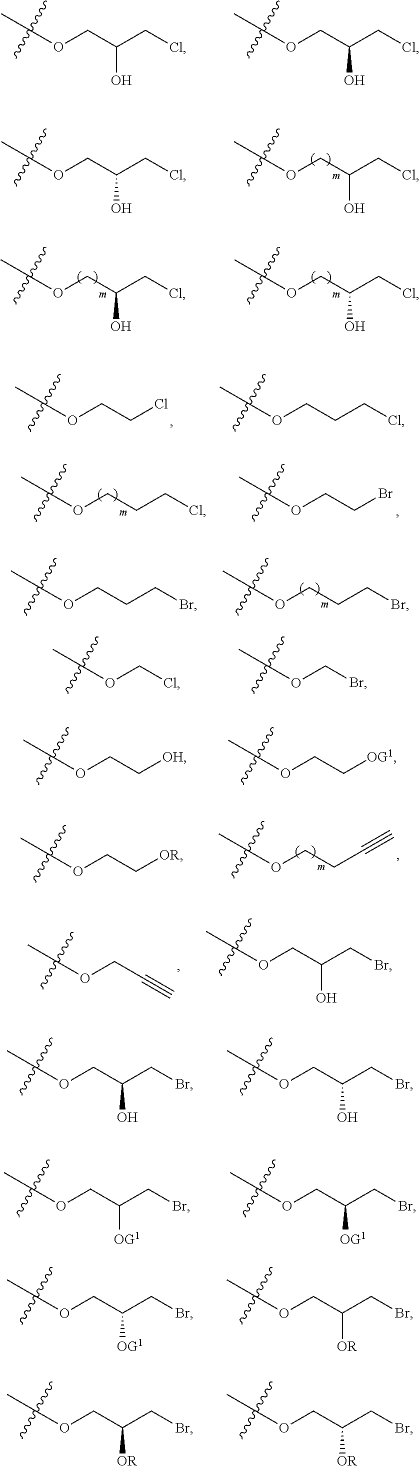 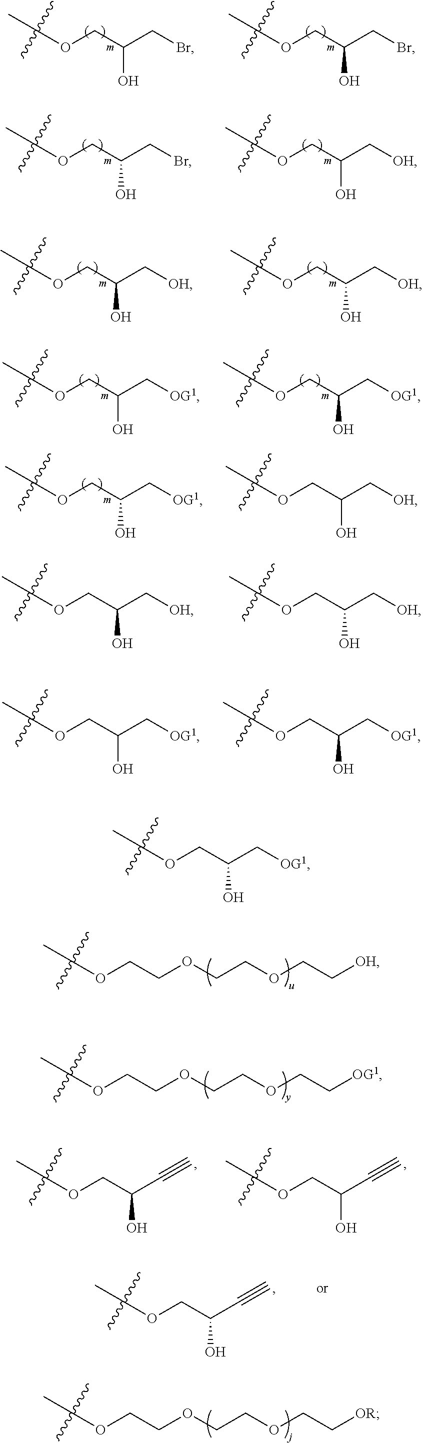
m is 0, 1, 2, 3,4, 5, 6, 7 or 8;
each of u, y and j is independently 0, 1, 2, 3, 4, 5, 6 or 7;
X is selected from the group consisting of: C═O, O, S, and SO;
each Z is independently selected from: N; CG¹; CH; CF; CCl; CBr; and CI; and
each G¹ is a linear or branched, substituted or unsubstituted, saturated or unsaturated C₁-C₁₀ alkyl, wherein the optional substituent may be selected from the group consisting of: oxo; Or; COOH; OH; F; Cl; Br; I; NH₂; CN; SH; SO₃H; CONH₂; OPO₃H₃; and NO₂.

41. A pharmaceutical composition comprising the compound of claim 1 and a pharmaceutically acceptable carrier.

42. A method for modulating androgen receptor activity, the method comprising administering a compound having a structure of Formula I: 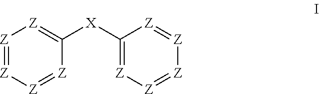
or a pharmaceutically acceptable salt thereof,
wherein:
X is a single bond, C═O, C(OH)₂, C(OR¹)₂, C(OH)(OR¹), C(OR¹)(OR²), O, S, SO, SO₂, C═NOH, C═N)R¹, CHNH₂, CHNHR¹, CHNHR², CHN(R¹)₂, CHN(R²)₂, or CHNR¹R²;
each R¹ is independently linear or branched, substituted or unsubstituted, saturated or unsaturated C₁-C₁₀ alkyl, and each R² is independently C₁-C₁₀ acyl, or two of the R¹ groups are joined to form a cyclic ketal, wherein the optional substituent may be selected from the group consisting of oxo, OJ′″, COOH, R³, OH, OR³, F, Cl, Br, I, NH₂, NHR³, N(R³)₂, CN, SH, SR³, SO₃H, SO₃R³, SO₂R³, OSO₃R³, OR⁶, CO₂R³, CONH₂, CONHR³, CONHR⁶, CON(R³)₂, NHR⁶, OPO₃H₃, CONR³R⁶, NR³R⁶, and NO₂;
each R³ is independently unsubstituted C₁-C₁₀ alkyl;
each R⁶ is independently C₁-C₁₀ acyl;
at least one Z of one aromatic ring is independently C-Q, at least one Z of the other aromatic ring is independently C-T, CF, CCl, CBr, CI, COH, CG¹, COG¹, CNH₂, CNHG¹, CNG¹₂, COSO₃H, COPO₃H₂, CSG¹, CSOG¹, or CSO₂G¹ and each remaining Z is independently C-T, N, CH, CF, CCl, CBr, CI, COH, CG¹, COG¹, CNH₂, CNHG¹, CN G¹)₂, COSO₃H, COPO₃H₂, CSG¹, CSOG¹, or CSO₂G¹;
Q is 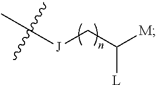
J is G¹, O, CH₂, CHG¹, CG¹₂, S, NH, NG¹, SO, SO₂, or NR;
M is H, Cl, Br, CH₂Cl, CHCl₂, CCl₃, CH₂Br, CHBr₂, CBr₃, or C≡CH;
L is H or A-D;
A is O, S, NH, NG¹, N⁺H₂, or N⁺HG¹;
D is H, G¹, R, 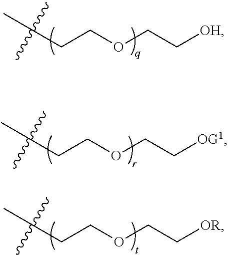
or a moiety selected from TABLE 1;
each of q, r and t is independently 0, 1, 2, 3, 4, 5, 6 or 7;
n is 0, 1, 2, 3, 4, 5, 6, 7 or 8;
T is 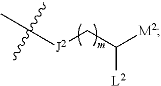
J² is G¹, O, CH₂, CHG¹, CG¹₂, S, NH, NG¹, SO, SO₂, or NR;
M² is H, CH₃, Cl, Br, CH₂Cl, CHCl₂, CCl₃, CH₂Br, CHBr₂, CBr₃, CH₂OH, CH₂OJ″, G¹, CH₂OG¹, CH₂OR, CH₂OG¹OG¹′, G¹OG¹′ G¹OG¹′OG¹″, CH₂SG¹, CH₂NH₂, CH₂NHG¹, CH₂NG¹₂, or C≡CH;
L² is H or A²-D²;
A² is O, S, SO, SO₂, NH, NG¹, N⁺H₂, or N⁺HG¹;
D² is H, G¹, R, 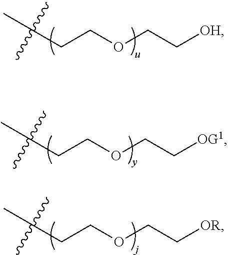
or a moiety selected from TABLE 1;
each of u, y and j is independently 0, 1, 2, 3, 4, 5, 6 or 7;
m is 0, 1, 2, 3,4,5,6,7 or 8;
each of J″ and J′″ is independently a moiety selected from TABLE 1;
each G¹ G¹′ and G¹′″ is independently linear or branched, or aromatic cyclic or non-aromatic cyclic, substituted or unsubstituted, saturated or unsaturated C₁-C₁₀ alkyl, wherein the optional substituent may be selected from the group consisting of oxo, OJ′″, COOH, R⁴, OH, OR⁴, F, Cl, Br, I, NH₂, NHR⁴, NR⁴₂, CN, SH, SR⁴, SO₃H, SO₃R⁴, SO₂R⁴, OSO₃R⁴, OR^{S}, CO₂R⁴, CONH₂, CONHR⁴, CONHR⁵, CONR⁴₂, NHR⁵, OPO₃H₃, CONR⁴R⁵, NR⁴R⁵, and NO₂;
each R⁴ is independently unsubstituted C₁-C₁₀ alkyl;
each R⁵ is independently C₁-C₁₀ acyl; and
R is C₁-C₁₀ acyl.

43-82. (canceled)

83. The method of claim 42, wherein the modulation of androgen receptor (AR) activity is for the treatment of one or more of the following: prostate cancer; breast cancer; ovarian cancer; endometrial cancer; hair loss; acne; hirsutism; ovarian cysts; polycystic ovary disease; precocious puberty; and age-related macular degeneration.

84-85. (canceled)

86. A pharmaceutical composition comprising a compound having a structure of Formula I as recited in claim 83 and a pharmaceutically acceptable carrier.
